(19) United States
(12) Patent Application Publication
(10) Pub. No.: US 2025/0101458 A1
(43) Pub. Date: Mar. 27, 2025

(54) OPTIMIZED AAV-BASED VACCINE

(71) Applicants: Massachusetts Eye and Ear infirmary, Boston, MA (US); The Schepens Eye Research Institute, Inc., Boston, MA (US)

(72) Inventors: Luc H. Vandenberghe, Weston, MA (US); Nerea Zabaleta Lasarte, Boston, MA (US); Urja Achal Bhatt, Boston, MA (US)

(73) Assignees: Massachusetts Eye and Ear infirmary, Boston, MA (US); The Schepens Eye Research Institute, Inc., Boston, MA (US)

(21) Appl. No.: 18/730,269

(22) PCT Filed: Jan. 19, 2022
(86) PCT No.: PCT/US2022/013015

§ 371 Date: Jul. 18, 2024

(51) Int. Cl. C12N 15/86; A61K 39/00; A61P 31/14; C07K 14/005

(52) U.S. Cl. CPC C12N 15/86; A61P 31/14; C07K 14/005; A61K 39/00; A61K 2039/53; C12N 2750/14122; C12N 2750/14143; C12N 2750/14151; C12N 2770/20034; C12N 2830/48; C12N 2830/50

(57) ABSTRACT

The present application relates to compositions and methods for eliciting an immune response in a subject using an Adeno-Associated Virus (AAV) AAV11 vector comprising an AAV11 capsid protein and a nucleic acid encoding a transgene operably linked to a promoter, wherein the transgene encodes an immunogenic polypeptide. Further disclosed are immunogenic polypeptides that are used for the compositions and methods.

### BACKGROUND

Vaccination has been shown to provide protection against certain infections and some cancers. There is a need for vaccines that are more effective against a larger number of infections and cancers.

### SUMMARY

AAV-based vaccines have been reported to have pre-clinical efficacy. For example, AAV-based COVID19 (AAVCOVID) vaccine candidates have shown durable and protective immunity in non-human primate (NHP) models upon immunization with a single dose. As a result, neutralizing antibody titers have been maintained at peak levels for at least 1-year post-immunization, and have continued to hold levels at 16 months. Such vaccines can leverage established manufacturing capacity in the industry, which can be scaled. Moreover, studies have indicated that the vaccine product can remain stable for 1 month at room-temperature. However, there remains a need to optimize AAV-based vaccines to potentially reduce the dosing required and to potentially improve potency. There also remains a need for a fast and efficiently adaptable platform in view of the potential for variants of concern.

Accordingly, described herein are compositions and methods for eliciting an immune response in a subject using an optimized AAV-base vaccine.

In some aspects, the disclosure provides an AAV11 vector comprising at least one AAV11 capsid protein and a nucleic acid encoding a transgene operably linked to a promoter, wherein the transgene encodes an immunogenic polypeptide. In some embodiments, the AAV11 viral capsid protein has an amino acid sequence at least 95% identical to SEQ ID NO: 1. In some embodiments, the AAV11 viral capsid protein has an amino acid sequence that is SEQ ID NO: 1. In some embodiments, the AAV11 viral capsid protein has an amino acid sequence comprising at least 2 amino acid substitutions compared to SEQ ID NO: 1. In some embodiments, the AAV11 viral capsid protein has an amino acid sequence comprising at least 2 amino acid deletions compared to SEQ ID NO: 1. In some embodiments, the AAV11 viral capsid protein has an amino acid sequence comprising at least one amino acid insertion compared to SEQ ID NO: 1. In some embodiments, the AAV11 viral capsid protein has an amino acid sequence comprising at least five amino acid insertions compared to SEQ ID NO: 1. In some embodiments, the AAV11 viral capsid protein has an amino acid sequence comprising at least seven amino acid insertions compared to SEQ ID NO: 1. In some embodiments, the AAV11 viral capsid protein has an amino acid sequence comprising at least ten amino acid insertions compared to SEQ ID NO: 1.

In some embodiments, the nucleic acid further comprises other regulatory elements. In some embodiments, the other regulatory elements comprise a Woodchuck Hepatitis Virus (WHP) Posttranscriptional Regulatory Element (WPRE). In some embodiments, the nucleic acid further comprises a polyadenylation (polyA) sequence. In some embodiments, the poly A sequence is a short synthetic polyA (SPA) sequence.

In some embodiments, the immunogenic polypeptide is from a virus selected from the group consisting of Adenoviruses, Arenaviruses, Arteriviruses, Birnaviruses, Bunyaviruses, Caliciviruses, Coronaviruses, Ebola viruses, Filoviruses, Flaviviruses, Hepadnaviruses, Herpesviruses, Human immunodeficiency virus (HIV), Human papilloma virus (HPV), Influenza viruses, Orthomyxoviruses, Paramyxoviruses, Picornaviruses, Parvoviruses, Papovaviruses, Pneumoviruses, Poxviruses, Rhabdoviruses, Reoviruses, Retroviruses, Rotaviruses, and Togaviruses. In some embodiments, the immunogenic polypeptide is from a microorganism selected from a species selected from Acinetobacter spp., including, for example, Acinetobacter baumannii; Bacillus spp.; Bartonella spp., including, for example, Bartonella henselae; Bordetella spp.; Borelia spp., including, for example, Borelia burgdorferi; Brucella spp., including, for example, Brucella melitensis; Camplybacter spp., including, for example, Camplybacter jejuni; Chlamydia spp., including, for example, Chlamydia pneumoiae; Clostridium spp., including, for example, Clostridium botulinum; Corynebacterium spp., including, for example, Corynebacterium amycolatum; Escherichia spp., including, for example, E. coli 0157: H7; Ehrlichia spp., including, for example, Ehrlichia chaffeensis; Enterococcus spp., including, for example, Enterococcus faecalis or Enterococcus faecium; Francisella spp., including, for example, Francisella tularensis; Haemophilus spp., including, for example, Haemophilus influenza; Helicobacter spp., including, for example, Helicobacter pylori; Klebsiella spp., including, for example, Klebsiella pneumonia; Legionella spp., including, for example, Legionella pneumophila; Leptospira spp., including, for example, Leptospira interrogans; Listeria spp., including, for example, Listeria monocytogenes; Mycobacterium spp., including, for example, Mycobacterium tuberculosis; Mycoplasma spp., including, for example, Mycoplasma pneumonia; Neisseria spp., including, for example, Neisseria gonorrhoeae; Parachlamydia spp.; Salmonella spp., including, for example, Salmonella enterica; Shigella spp., including, for example, Shigella sonnei; Staphylococcus spp., including, for example, Staphylococcus aureus; Streptococcus spp., including, for example, Streptococcus pneumonia or Streptococcus pyogenes; Vibrio spp., including, for example, Vibrio vulnificus; and Yersinia spp., including, for example, Yersinia pestis. In some embodiments, the immunogenic polypeptide is from a parasite.

In some embodiments, the immunogenic polypeptide comprises a viral antigen. In some embodiments, the viral antigen comprises a coronavirus spike protein or a fragment of a coronavirus spike protein. In some embodiments, the viral antigen comprises a coronavirus receptor-binding domain (RBD) or a fragment of a coronavirus RBD. In some embodiments, the coronavirus is a SARS-COV-2 virus.

In some embodiments, the immunogenic polypeptide comprises a bacterial antigen. In some embodiments, the immunogenic polypeptide comprises a parasitic antigen. In some embodiments, the immunogenic polypeptide comprises a fungal antigen.

In some embodiments, the immunogenic polypeptide comprises a cancer antigen. In some embodiments, the cancer antigen is NY-ESO-1, HER2, HPV16 E7, CEA, WT1, MART-1, gp100, tyrosinase, URLC10, VEGFR1, VEGFR2, survivin, MUC1, or MUC2.

In some embodiments, the promoter is selected from the group consisting of a CAG promoter, an EF1 alpha promoter, a p5 promoter, a p19 promoter, a p40 promoter, a SV40 promoter, an elongation factor short (EFS) promoter, a muscle creatine kinase (MCK) promoter, a cytomegalovirus (CMV) promoter, or a minimal CMV (mini-CMV) promoter. In some embodiments, the promoter is a mini-CMV promoter. In some embodiments, the promoter comprises a mini-CMV enhancer. In some embodiments, the promoter is a mini-CMV enhancer and promoter. In some embodiments, the mini-CMV promoter has at least 90% identity to SEQ ID NO: 12. In some embodiments, the mini-CMV promoter is SEQ ID NO: 12. In some embodiments, the mini-CMV enhancer has at least 90% identity to SEQ ID NO: 13. In some embodiments, the mini-CMV enhancer is SEQ ID NO: 13. In some embodiments, the mini-CMV promoter has at least 90% identity to SEQ ID NO: 14. In some embodiments, the mini-CMV promoter is SEQ ID NO: 14.

In some embodiments, the nucleic acid further comprises an intron. In some embodiments, the intron is located downstream of the promoter. In some embodiments, the promoter is a CMV promoter and the nucleic acid further comprises an intron located downstream of the promoter.

In some aspects, the disclosure provides a composition comprising an AAV11 vector as described herein, and a pharmaceutically acceptable carrier. In some aspects, the disclosure provides a vaccine comprising an AAV11 vector as described herein.

In some aspects, the disclosure provides a method of eliciting an immune response in a subject comprising administering to the subject a composition as described herein. In some aspects, the disclosure provides a method of treating or preventing a disease in a subject comprising administering to the subject a composition as described herein.

In some embodiments, the composition is administered to the subject only once. In some embodiments, the composition is administered to the subject more than once. In some embodiments, the composition administered to the subject comprises a viral dosage of 10⁸ to 10¹³ genome copies. In some embodiments, the composition is administered to the subject via a route of administration selected from the group consisting of intramuscular, intravenous, subcutaneous, rectal, intravaginal, parenteral, oral, sublingual, intratracheal, or and intranasal.

In some embodiments, the subject is a mammal. In some embodiments, the subject is selected from the group consisting of a human, a non-human primate, a rodent, an exotic animal, a companion animal, and livestock. In some embodiments, the subject is at risk of developing an infection or cancer. In some embodiments, the subject is at risk of developing a disease selected from the group consisting of SARS-COV-1 and SARS-COV-2 (COVID-19).

In some aspects, the disclosure provides a method of manufacturing an AAV11 vector, comprising (i) transfecting a host cell expressing an AAV11 capsid protein with a nucleic acid encoding an immunogenic polypeptide as described herein operably linked to a promoter, and (ii) culturing the host cell under conditions in which AAV11 vectors comprising at least one AAV11 viral capsid protein as described herein comprising the nucleic acid are produced. In some embodiments, the host cell is a producer cell. In some embodiments, the host cell is a packaging cell.

In some aspects, the disclosure provides a method of manufacturing an AAV11 vector, comprising (i) transfecting a producer cell with a nucleic encoding an AAV11 capsid protein and another nucleic acid encoding an immunogenic polypeptide operably linked to a promoter, and (ii) culturing the producer cell under conditions in which AAV11 vectors comprising at least one AAV11 viral capsid protein as described herein comprising the nucleic acid are produced.

In some embodiments, the antigen plasmid further comprises a Woodchuck Hepatitis Virus (WHP) Posttranscriptional Regulatory Element (WPRE). In some embodiments, the viral antigen comprises a coronavirus spike protein, or a fragment of a coronavirus spike protein. In some embodiments, the viral antigen comprises a coronavirus receptor-binding domain (RBD), or a fragment of a coronavirus RBD. In some embodiments, the coronavirus is a SARS-COV-2 virus.

In some embodiments, the promoter is selected from the group consisting of a CAG promoter, an EF1 alpha promoter, a p5 promoter, a p19 promoter, a p40 promoter, a SV40 promoter, an elongation factor short (EFS) promoter, a muscle creatine kinase (MCK) promoter, a cytomegalovirus (CMV) promoter, and a minimal CMV (mini-CMV) promoter.

In some embodiments, the antigen plasmid comprises a nucleic acid sequence at least 90% identical to any one of SEQ ID NOs: 3-6. In some embodiments, the antigen plasmid comprises a nucleic acid sequence that is any one of SEQ ID NOs: 3-6.

In some embodiments, the cell is an insect cell. In some embodiments, the insect cell is a baculovirus cell. In some embodiments, the cell is a mammalian cell.

In some embodiments, the nucleic acid encoding an immunogenic polypeptide is expressed from a recombinant AAV genome.

These and other aspects of the disclosure, as well as various advantages and utilities will be apparent with reference to the Detailed Description. Each aspect of the disclosure can encompass various embodiments as will be understood. All documents identified in this application are incorporated in their entirety herein by reference.

### BRIEF DESCRIPTION OF THE DRAWINGS

FIG. 1 shows a longitudinal analysis of pseudovirus neutralization (international units (IU)/mL) in rhesus macaques vaccinated with 10¹²gc of AC1 and AC3 (n=2).

FIGS. 2A-2I show exemplary results that low doses of first generation AAVCOVID only partially protect cynomolgus macaques. Cynomolgus macaques vaccinated with 10¹¹gc of AC1 and AC3 (n=6) and controls were challenged with 10⁵ pfu of SARS-COV-2 (BetaCoV/France/IDF/0372/2020) on week 9.5 after vaccination. FIG. 2A is a graph of the concentration of RBD-binding IgG (arbitrary units (AU)/mL) in vaccinated and control animals over time. FIG. 2B is a graph of pseudovirus neutralization (IU/mL) over time. FIG. 2C is a graph of IFN-γ spot-forming units (SFU) per million PBMC as measured by ELISPOT. FIGS. 2D-2E are graphs of copies of SARS-COV-2 viral RNA (FIG. 2D) and subgenomic RNA or sgRNA (FIG. 2E) over time. The results are shown as quantification (copies/mL) after challenge in nasopharyngeal swabs. FIGS. 2F-2G are graphs of copies of SARS-COV-2 viral RNA (FIG. 2F) and sgRNA (FIG. 2G) over time. The results are shown as quantification (copies/mL) 3 days after challenge in bronchoalveolar lavage (BAL). FIG. 2H is a graph of the measurement of lung lymph node activation by PET as mean standardized uptake values (SUV mean) before and after challenge. FIG. 21 is a graph of the lung histopathology score 30-35 days after challenge. The graphs in FIGS. 2A-2H used Mann-Whitney test to compare vaccinated groups with controls. *p<0.05. **p<0.01. Grey shaded areas correspond to post-challenge timepoints. The graph in FIG. 2I used Tukey's test. ****p<0.0001.

FIGS. 3A-3E show exemplary results for the second generation AAVCOVID platform optimized for capsid. C57BL/6 mice (7-8 weeks old) were injected IM with two doses (10¹⁰ gc and 10¹¹ gc) of AC1 or AAV11-Spp, n=10 (5/gender). FIG. 3A is a graph of SARS-COV-2 RBD-binding IgG titers (reciprocal serum dilution) over time. FIG. 3B is a graph of pseudovirus neutralizing titers (reciprocal serum dilution) over time. FIGS. 3C-3D are graphs of spot-forming units (SFU) detected by IFN-γ (FIG. 3C) or IL-4 (FIG. 3D) ELISPOT in splenocytes harvested 10 weeks after vaccination with 10¹⁰ gc of AC1 or AAV11-Spp and stimulated with Spike peptides. FIG. 3E is a graph of the quantification of vector genome copies (genome copies/diploid genome or gc/dg) in the right gastrocnemius (right gastroc) or injection site, left gastrocnemius (left gastroc) or contralateral muscle, liver, and spleen on week 10 (n=5). The dotted lines indicate the lower detection limit of the assays. Data are represented as geometric mean±SD. Unpaired t test with Welch's correction was used for comparison of animals with same dose of AAV11-Spp and AC1.

FIGS. 4A-4D show exemplary results for the second generation AAVCOVID platform optimized for promoter. FIG. 4A are schematics of new cassettes. SV40: simian virus 40 promoter and polyadenylation signal. ITR: inverted terminal repeat. Spp: prefusion stabilized Spike. SPA: synthetic polyA. EFS: elongation factor short promoter. miniCMV: minimal CMV promoter. FIG. 4B is a graph of transgene mRNA expression (RBD copies (cp)/GAPDH copics) 7 days after IM administration of 10¹¹gc in C57BL/6 animals (n=5 females). Data are represented as mean±SD. FIG. 4C is a graph of RBD-binding antibody titers in C57BL/6 animals (n=5-10 females) at three different doses. FIG. 4D is a graph of IFN-γ ELISPOT on day 56 after vector administration. The graphs in FIGS. 4A-4B used Kruskal Wallis test and Dunn's posttest. The graph in FIG. 4C used Mann Whitney test. *p<0.05. **p<0.01, ****p<0.0001.

FIGS. 5A-5D show exemplary results of the robust and rapid programmability of ACM with VOC antigen. FIG. 5A is a schematic representation of ACM-Beta and ACM-Delta recombinant genomes. ITR: inverted terminal repeat. Spp: prefusion stabilized Spike. SPA: synthetic polyA. miniCMV: minimal CMV promoter. FIG. 5B is a graph of VOC pseudovirus neutralization on day 56 in C57BL/6 animals vaccinated with 10¹¹gc of ACM-Beta (n=4). FIG. 5C is a graph of self-RBD-binding antibody titers on day 14 in C57BL/6 animals vaccinated with 10¹¹gc of ACM1, ACM-Beta or ACM-Delta (n=5). FIG. 5D is a graph of different pseudovirus (Wuhan, Beta and Delta) neutralization in animals vaccinated with different candidates on day 28 after vaccination (n=5).

FIGS. 6A-6F show exemplary results that ACM-Beta protects from Beta SARS-COV-2 challenge in cynomolgus macaques at low dose. Cynomolgus macaques vaccinated with 10¹¹gc of ACM-Beta (n=5) and controls (n=6) were challenged with 10⁵ pfu of Beta SARS-COV-2 VOC on week 7.5 after vaccination. FIG. 6A is a graph of beta RBD-binding IgG concentration (arbitrary units (AU)/mL) in vaccinated animals. FIG. 6B is a graph of ACE2 binding inhibition assay (AU/mL) in vaccinated animals. FIG. 6C is a graph of beta Spike pseudovirus neutralizing antibody titer (EC50) in vaccinated animals. FIG. 6D is a graph of IFN-γ spot-forming units (SFU) per million PBMC measured by ELISPOT. FIGS. 6E-6F are graphs of beta SARS-COV-2 viral RNA (FIG. 6E) and subgenomic RNA or sgRNA (FIG. 6F) quantification (copies/mL) after challenge in nasopharyngeal swabs and tracheal swabs during 10 days after the challenge and in bronchoalveolar lavage (BAL) on day 3. Mann Whitney test. *p<0.05, **p<0.01.

FIGS. 7A-7D show exemplary results that AAVCOVID induces potent CD4+ T cell responses. ICS analysis of PBMCs extracted from cynomolgus macaques vaccinated with first and second generation AAVCOVID vaccines on weeks 9 and 6, respectively. FIGS. 7A-7B are graphs of IFN-γ-secreting CD4+ T cells before and after vaccination in animals vaccinated with two doses of AC1 (FIG. 7A) (n=6 per group), low dose of AC3 (FIG. 7A) (n=6) and ACM-Beta (FIG. 7B) (n=5). FIGS. 7C-7D are pie charts showing the percentage of Th-specific cytokine-secreting CD4+ T cells (upper row) and percentage of Th1 cells secreting 1, 2 or 3 cytokines (lower row) on week 9 (FIG. 7C) and week 6 (FIG. 7D).

FIGS. 8A-8E show results related to FIGS. 2A-2I, providing exemplary results that low doses of first generation AAVCOVID only partially protect cynomolgus macaques. Cynomolgus macaques vaccinated with 10¹¹gc of AC1 and AC3 (n=6) and controls challenged with 10⁵ pfu of SARS-COV-2 (BetaCoV/France/IDF/0372/2020) on week 9.5 after vaccination. FIG. 8A is a graph of spike-binding IgG (AU/mL). FIG. 8B is a graph of ACE2 inhibition assay (AU/mL). FIGS. 8C-8D are graphs of SARS-COV-2 viral RNA (FIG. 8C) and subgenomic RNA or sgRNA (FIG. 8D) quantification (copies/mL) after challenge in tracheal swabs. FIG. 8E are exemplary CT scores in lungs of control and vaccinated animals before and after challenge. Scores were calculated based on lesion type (scored from 0 to 3) and lesion volume (scored from 0 to 4) for each lobe.

FIGS. 9A-9D show results related to FIGS. 3A-3E, are exemplary data for the second generation AAVCOVID platform optimized for capsid. Balb/c mice (7-8 weeks old) were injected IM with two doses (10¹⁰ gc and 10¹¹ gc) of AC1 or AAV11-Spp, n=5 females. FIG. 9A is a graph of SARS-COV-2 RBD-binding IgG titers at different time post vaccination. FIG. 9B is a graph of pseudovirus neutralizing titers at week 5 post vaccination. FIGS. 9C-9D are graphs of spot-forming units (SFU) detected by IFN-γ (FIG. 9C) or IL-4 (FIG. 9D) ELISPOT in splenocytes extracted from animals 5 weeks after vaccination with two doses of AC1 or AAV11-Spp and stimulated with peptides spanning SARS-COV-2 spike protein for 48 h. The dotted lines indicate the lower detection limit of the assays. Data are represented as geometric mean±SD. Mann-Whitney test for comparison of animals with same dose of AAV11-Spp and AC1. *p<0.05.

FIGS. 10A-10D show results related to FIGS. 4A-4D, are exemplary data for the second generation AAVCOVID platform optimized for promoter. FIG. 10A is a schematic for an ACC1 candidate. ITR: inverted terminal repeat. Spp: prefusion stabilized Spike. SPA: synthetic polyA. CMV: CMV promoter. FIG. 10B is a representation of spike expression analyzed by WB in Huh7 cells infected with 5×10⁵ MOI of vaccine candidates. FIG. 10C is a graph of transgene mRNA expression (RBD copies (cp)/GAPDH copies) 7 days after IM administration of 10¹¹gc in C57BL/6 animals (n=5 females). Data are represented as mean±SD. FIG. 10D is a graph of RBD-binding antibody titers on day 56 in C57BL/6 animals (n=5 females) vaccinated with two doses. FIGS. 10C-10D used Mann Whitney test. *p<0.05, **p<0.01.

FIGS. 11A-11C show results related to FIGS. 6A-6F are exemplary results that ACM-Beta protects from Beta SARS-COV-2 challenge in cynomolgus macaques at low dose.

Cynomolgus macaques vaccinated with 10¹¹gc of ACM-Beta (n=5) and controls (n=6) were challenged with 10⁵ pfu of Beta SARS-COV-2 VOC on week 7.5 after vaccination. FIG. 11A is a graph of VOC RBD-binding IgG titer in vaccinated animals on week 6 after vaccination. FIG. 11B is a graph of VOC RBD-ACE2 binding inhibition assay on week 6 after vaccination. FIG. 11C is a graph of VOC pseudovirus neutralization assay in vaccinated animals on week 9 after vaccination (10 days after challenge).

FIG. 12 shows exemplary results that low IM dose of CD platform biodistributes primarily focally, with limited expression in liver. Cynomolgus macaques (n=6) received single dose IM injection, at week 9 SARS-COV-2 1E5 PFU IN/IT, and necropsy at week 14 post immunization, ddPCR readout for vg RBD target sequence. At proposed 1E11 gc dose, primary distribution in NHP is focal (thigh muscle injection site and lymph node with limited distribution to liver. Abbreviations: gc-genome copies; dg-diploid genome.

FIG. 13 shows graphs of exemplary results of RBD binding titers comparing AC1 and ACM1 over 100× range in C57BL/6 female mice. ACM1 in C57BL/6 female mice elicits a faster and more potent response with a lower dose. Mann Whitney test was used.

FIG. 14 shows graphs of VOC immunogenicity of ACM-Beta at 1011 gc. AC1 (with Spike Wuhan strain) was administered at 10× higher concentration than ACM-Beta.

FIG. 15 shows graphs of potent cellular immune responses in NHP, and comparison of AC platform with ACM in NHP. Potent IFN-γ cellular responses were elicited in cynomolgus macaques.

FIG. 16 shows graphs demonstrating no desensitization against Spike antigen was observed in mice. Antibody responses can be boosted 6 months after vaccination in mice with a heterologous vector. Antibody responses increased after boosting with a different vector carrying the prefusion stabilized Spike. First dose: week 0, AC1 or AC3 1010 gc; booster: week 26, AAV1-SU.S. Plant Pat. No. 1,011gc.

FIG. 17 shows a graph of low anti-vector antibody responses in rhesus. Slow kinetics of humoral responses against the vector. Antibodies against AAVrh32.33 at 1E12 gc stabilize starting week 12. No cross-reactivity was observed with serotypes 1, 2, 5, 8 and 9.

FIGS. 18A-18D show exemplary results that immunogenicity of ubiquitous and muscle restricted AC1 vaccine. FIG. 18A shows a schematic of AC1 and AC1-MCK genomes. FIG. 18B shows a graph of spike RBD binding IgG in sera of C57BL/6 female mice vaccinated with 1011 gc total dose of AC1 or AC1-MCK. FIG. 18C shows a graph or IFN-γ secreting splenocytes on day 35. FIG. 18D shows a graph transgene expression in the injection site on day 7 after injection. Mean±SD, Mann Whitney test. *p<0.05, **p<0.01.

FIG. 19 is a graph showing the levels of RBD-specific IgG antibodies in female vaccinated mice over time and in response to dose.

FIG. 20 is a graph showing the levels of spike-neutralizing antibodies in female vaccinated mice at day 35.

FIG. 21 is a graph showing the levels of INF-gamma and IL-4-secreting T cells produced in female vaccinated mice following stimulation with SARS-COV-2 peptides.

FIG. 22 is a graph showing the levels of RBD-specific IgG antibodies in male and female vaccinated mice over time and in response to dose.

FIG. 23 is a graph showing the levels of spike-neutralizing antibodies in male and female vaccinated mice over time and in response to dose.

FIG. 24 is a graph showing the levels of INF-gamma-secreting T cells produced in male and female vaccinated mice (vaccinated at the lower level) following stimulation with SARS-CoV-2 peptides.

FIG. 25 is a graph showing the levels of IL-4-secreting T cells produced in male and female vaccinated mice (vaccinated at the lower level) following stimulation with SARS-COV-2 peptides.

FIG. 26 is a graph showing the biodistribution of each vector in male (left) and female (right) vaccinated mice (vaccinated at the higher dose).

FIG. 27 is a graph showing the liver biodistribution of each vector in male and female vaccinated mice (vaccinated at the higher dose).

FIG. 28 is a graph showing the levels of RBD-specific IgG antibodies in female vaccinated mice in response to dose on day 56.

FIG. 29 are graphs showing protection of non-human primates from infection with SARS-COV-2 challenge, based on viral replication.

FIG. 30 are schematics of candidates for AAV-COVID vaccines carrying a membrane-anchored or secreted Spike antigen.

FIG. 31 are graphs demonstrating the 11-month durability of immunogenicity in rhesus.

FIG. 32 is an alignment between AAV11 (SEQ ID NO:1) and Rh32.33 (SEQ ID NO:3) VP1 sequences showing conserved regions as well as a number of hypervariable regions (HVR; boxed and numbered).

### DETAILED DESCRIPTION

Described herein are compositions and methods for eliciting an immune response in a subject. In some aspects, an adeno-associated viral vector (AAV)-based vaccine platform is provided. As described herein, AAV vectors (e.g., recombinant AAV viral particles) can be used to deliver a nucleic acid encoding an immunogenic polypeptide. Nucleic acids for delivery by AAV vectors generally do not encode for any viral gene, and typically are made up of a single-stranded (ss) DNA molecule containing an expression cassette (e.g., encoding an immunogenic polypeptide) flanked by viral inverted terminal repeats (ITRs). AAV vectors have the ability to effectively transduce a number of different tissues in vivo.

### Adeno-Associated Virus (AAV) and AAV11

Adeno-associated viruses (AAVs) are small viruses that infect humans and some non-human primate (NHP) species. AAVs belong to the genus Dependoparvovirus, and the family Parvoviridae. AAVs are small (20 nm) replication-defective, non-enveloped DNA viruses. The small (4.8 kb) ssDNA genome consists of two open reading frames, Rep and Cap, flanked by two 145 base ITRs. These ITRs base pair to allow for synthesis of the complementary DNA strand. Rep and Cap are translated to produce multiple distinct proteins (e.g., Rep78, Rep68, Rep52, and Rep40, required for the AAV life cycle; and VP1, VP2, and VP3, the capsid proteins). When constructing a nucleic acid to be delivered using AAV, the exogenous nucleic acid (e.g., an immunogenic polypeptide transgene) is placed between the two ITRs, and Rep and Cap typically are supplied in trans.

AAVs typically cause a very mild immune response, but generally are thought not to cause disease. AAVs often can infect both dividing and quiescent cells, and can persist in an extrachromosomal state with very little to no integration into the genome of the host cell, although with the native virus, integration of virally carried genes into the host genome does occasionally occur.

A number of different serotypes of AAV have been identified. These serotypes differ in their tropism, or the types of cells they infect, making AAV a very useful system for preferentially transducing specific cell types. Researchers have further refined the tropism of AAV through pseudotyping, or the mixing of a capsid and genome from different viral serotypes. For example, AAV2/5 indicates a virus containing the genome of Serotype 2 packaged in the capsid from Serotype 5. Use of these pseudotyped viruses can improve transduction efficiency, as well as alter tropism.

In some aspects, the disclosure provides an AAV11 vector comprising at least one AAV11 capsid protein and a nucleic acid encoding a transgene operably linked to a promoter. In some aspects, the disclosure provides a vaccine platform comprising an AAV11 capsid protein and a nucleic acid encoding a transgene operably linked to a promoter. The AAV11 serotype was isolated from the liver of cynomolgus monkeys, and the VP1 sequence of AAV11 can be found in SEQ ID NO: 1 and at GenBank Accession No. AAT46339.1.

In some embodiments, the AAV11 viral capsid protein has an amino acid sequence at least 90% identical to SEQ ID NO: 1. In some embodiments, the AAV11 viral capsid protein has an amino acid sequence at least 91% identical to SEQ ID NO: 1. In some embodiments, the AAV11 viral capsid protein has an amino acid sequence at least 92% identical to SEQ ID NO: 1. In some embodiments, the AAV11 viral capsid protein has an amino acid sequence at least 93% identical to SEQ ID NO: 1. In some embodiments, the AAV11 viral capsid protein has an amino acid sequence at least 94% identical to SEQ ID NO: 1. In some embodiments, the AAV11 viral capsid protein has an amino acid sequence at least 95% identical to SEQ ID NO: 1. In some embodiments, the AAV11 viral capsid protein has an amino acid sequence at least 96% identical to SEQ ID NO: 1. In some embodiments, the AAV11 viral capsid protein has an amino acid sequence at least 97% identical to SEQ ID NO: 1. In some embodiments, the AAV11 viral capsid protein has an amino acid sequence at least 98% identical to SEQ ID NO: 1. In some embodiments, the AAV11 viral capsid protein has an amino acid sequence at least 98.5% identical to SEQ ID NO: 1. In some embodiments, the AAV11 viral capsid protein has an amino acid sequence at least 99% identical to SEQ ID NO: 1. In some embodiments, the AAV11 viral capsid protein has an amino acid sequence at least 99.1% identical to SEQ ID NO: 1. In some embodiments, the AAV11 viral capsid protein has an amino acid sequence at least 99.2% identical to SEQ ID NO: 1. In some embodiments, the AAV11 viral capsid protein has an amino acid sequence at least 99.3% identical to SEQ ID NO: 1. In some embodiments, the AAV11 viral capsid protein has an amino acid sequence at least 99.4% identical to SEQ ID NO: 1. In some embodiments, the AAV11 viral capsid protein has an amino acid sequence at least 99.5% identical to SEQ ID NO: 1. In some embodiments, the AAV11 viral capsid protein has an amino acid sequence at least 99.6% identical to SEQ ID NO: 1. In some embodiments, the AAV11 viral capsid protein has an amino acid sequence at least 99.7% identical to SEQ ID NO: 1. In some embodiments, the AAV11 viral capsid protein has an amino acid sequence at least 99.8% identical to SEQ ID NO: 1. In some embodiments, the AAV11 viral capsid protein has an amino acid sequence at least 99.9% identical to SEQ ID NO: 1. In some embodiments, the AAV11 viral capsid protein has an amino acid sequence that is SEQ ID NO: 1. In some embodiments, the AAV11 viral capsid protein has an amino acid sequence comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid substitutions compared to SEQ ID NO: 1. In some embodiments, the AAV11 viral capsid protein has an amino acid sequence comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid deletions compared to SEQ ID NO: 1. In some embodiments, the AAV11 viral capsid protein has an amino acid sequence comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid insertions compared to SEQ ID NO: 1.

In addition, the AAV11 capsid protein can be used in combination (e.g., cross-packaged) with, for example, AAV2, or another suitable AAV serotype. The AAV11 capsid protein shares around 65% of amino acids with AAV2, and phylogenetic analysis indicated that AAV11 is very closely related to rh32.33 (see for example U.S. Pat. Nos. 8,999,678; 10,301,648; and 10,947,561, all of which are incorporated by reference in their entirety). AAV11 appears to exhibit a different and broader tropism than AAV2, one of the most common AAV serotypes used, and, following administration, AAV11 has been observed in the brain, muscle, intestine, uterus, kidney, spleen, lung, heart and stomach. AAV11 transduces undifferentiated cells at a much lower efficiency than AAV2, but AAV11 transduces differentiated cells more efficiently than AAV2. Notably, antibodies against a number of other serotypes did not cross-neutralize AAV11. See, for example, Mori et al. (2004, Virology, 330:375-83) and Mori et al. (2008, Arch. Virol., 153:375-80).

### Promoters

In some aspects, the disclosure provides an AAV11 vector comprising at least one AAV11 capsid protein and a nucleic acid encoding a transgene operably linked to a promoter. The particular promoter used to drive expression of the immunogenic polypeptide can allow for differential dosing of an AAV11-based vaccine composition as described herein. Suitable promoters for viral vector expression are known in the art and include, without limitation, CAG promoters, EF1 alpha promoters, p5 promoters, p19 promoters, p40 promoters, SV40 promoters, elongation factor short (EFS) promoters, muscle creatine kinase (MCK) promoters, cytomegalovirus (CMV) promoters, or minimal CMV (mini-CMV) promoters. The sequences of representative promoters are shown below.

SV40 promoter:
(SEQ ID NO: 10)
GGTGTGGAAAGTCCCCAGGCTCCCCAGCAGGCAGAAGTATGCAAAGCATGCATCTC
AATTAGTCAGCAACCAGGTGTGGAAAGTCCCCAGGCTCCCCAGCAGGCAGAAGTAT
GCAAAGCATGCATCTCAATTAGTCAGCAACCATAGTCCCGCCCCTAACTCCGCCCAT
CCCGCCCCTAACTCCGCCCAGTTCCGCCCATTCTCCGCCCCATGGCTGACTAATTTTT
TTTATTTATGCAGAGGCCGAGGCCGCCTCGGCCTCTGAGCTATTCCAGAAGTAGTGA
GGAGGCTTTTTTGGAGGCCTAGGCTTTTGCAAA
CMV promoter:
(SEQ ID NO: 11)
Tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggta
aatggcccgcctggcattatgcccagtacatgaccttatgggactttcctacttggcagtacatctacgtattagtcatcgctattaccatgg
tgatgcggttttggcagtacatcaatgggcgtggatagcggtttgactcacggggatttccaagtctccaccccattgacgtcaatgggagtt
tgttttggcaccaaaatcaacgggactttccaaaatgtcgtaacaactccgccccattgacgcaaatgggcggtaggcgtgtacggtgggagg
tctatataagcagagctggtttagtgaaccgt
mini-CMV enhancer and promoter:
(SEQ ID NO: 12)
cgttacataacttacggtaaatggcccgcctggctgaccgcccaacgacccccgcccattgacgtcaataatgacgtatgttcccatagtaa
cgccaatagggactttccattgacgtcaatgggtggagtatttacggtaaactgcccacttggcagtacatcaagtgtatcatatgccaagta
cgccccctattgacgtcaatgacggtaaatggcccgcctggcattatgcccagtacatgaccttatgggactttcctacttggcagtacatct
acgtattagtcatcgctattaccatgATACAGCTCCACCGCACATGCCACCCTCCGGATATATTCGTCT
CGAGCAAATCACTTGGCAGTCTAGCG
mini-CMV enhancer:
(SEQ ID NO: 13)
cgttacataacttacggtaaatggcccgcctggctgaccgcccaacgacccccgcccattgacgtcaataatgacgtatgttcccatagtaa
cgccaatagggactttccattgacgtcaatgggtggagtatttacggtaaactgcccacttggcagtacatcaagtgtatcatatgccaagta
cgccccctattgacgtcaatgacggtaaatggcccgcctggcattatgcccagtacatgaccttatgggactttcctacttggcagtacatct
acgtattagtcatcgctattaccatg
mini-CMV promoter:
(SEQ ID NO: 14)
ATACAGCTCCACCGCACATGCCACCCTCCGGATATATTCGTCTCGAGCAAATCACTT
GGCAGTCTAGCG
EFS promoter:
(SEQ ID NO: 15)
Taggtcttgaaaggagtgggaattggctccggtgcccgtcagtgggcagagcgcacatcgcccacagtccccgagaagttggggggag
gggtcggcaattgatccggtgcctagagaaggtggcgcggggtaaactgggaaagtgatgtcgtgtactggctccgcctttttcccgagg
gtgggggagaaccgtatataagtgcagtagtcgccgtgaacgttctttttcgcaacgggtttgccgccagaacacag
MCK promoter:
(SEQ ID NO: 16)
CCACTACGGGTCTAGGCTGCCCATGTAAGGAGGCAAGGCCTGGGGACACCC
GAGATGCCTGGTTATAATTAACCCcaacacctGCTGccccccccccccAACACCTGCTGCCTGA
GCCTgagcggttaCCCCACCCCGGTGCCTGGGTCTTAGGCTCTGTACACCATGGAGGAGA
AGCTCGCTCTAAAAATAACCCTGTCCCTGGTGGATCCTCCCTGGGGACAGCCCCTCC
TGGCTAGTCACACCCTGTAGGCTCCTCTATATAACCCAGGGGCACAGGGGCTGCCC
CCGGGTCAC

In some embodiments, the promoter is a CMV promoter. In some embodiments, the promoter is a full CMV promoter or a minimal CMV (mini-CMV) promoter. In some embodiments, the promoter is a mini-CMV promoter. In some embodiments, the promoter comprises a mini-CMV enhancer. In some embodiments, the promoter is a mini-CMV enhancer and promoter. In some embodiments, the mini-CMV promoter has at least 90% identity to SEQ ID NO: 12. In some embodiments, the mini-CMV promoter is SEQ ID NO: 12. In some embodiments, the mini-CMV enhancer has at least 90% identity to SEQ ID NO: 13. In some embodiments, the mini-CMV enhancer is SEQ ID NO: 13. In some embodiments, the mini-CMV promoter has at least 90% identity to SEQ ID NO: 14. In some embodiments, the mini-CMV promoter is SEQ ID NO: 14.

In some embodiments, the promoter is a tissue-specific or cell-specific promoter. In some embodiments, the promoter is a liver-specific promoter. In some embodiments, the promoter is a lung-specific promoter. In some embodiments, the promoter is an epithelial cell-specific promoter. In some embodiments, the promoter is an endothelial cell-specific promoter. In some embodiments, the promoter is a muscle-specific promoter. In some embodiments, the promoter is a muscle creatine kinase (MCK) promoter. In some embodiments, the promoter is a cardiac muscle-specific promoter. In some embodiments, the promoter is a reproductive tissue-specific promoter. In some embodiments, the promoter is an eye-specific promoter. In some embodiments, the promoter is a brain-specific promoter. In some embodiments, the promoter is a gastrointestinal tissue-specific promoter. In some embodiments, the promoter is an adipose tissue-specific promoter. In some embodiments, the promoter is a thyroid-specific promoter. Other examples of cell or tissue-specific promoters include, but are not limited to, the glucose-6-phosphatase (G6P) promoter, vitellogenin promoter, ovalbumin promoter, ovomucoid promoter, conalbumin promoter, ovotransferrin promoter, prolactin promoter, kidney uromodulin promoter, placental lactogen promoter, smooth-muscle SM22 promoter, including chimeric SM22alpha/telokin promoters, ubiquitin C promoter, Hsf2 promoter, murine COMP (cartilage oligomeric matrix protein) promoter, early B cell-specific mb-1 promoter, prostate specific antigen (PSA) promoter, exorh promoter and pineal expression-promoting element, neural and liver ceramidase gene promoters, PSP94 gene promoter/enhancer, promoter of the human FAT/CD36 gene, VL30 promoter, and IL-10 promoter.

In some embodiments, the promoter is an inducible promoter. Examples of inducible promoters include, but are not limited to, reproductive hormone induced promoters, antibiotic inducible promoters such as the tetracycline inducible promoter and the zinc-inducible metallothionine promoter, IPTG inducible promoters such as the Lac operator repressor system, ecdysone-based inducible systems, estrogen-based inducible systems, progesterone-based inducible systems, and CID-based inducible systems using chemical inducers of dimerization (CIDs).

### Immunogenic Polypeptides

Vaccination leverages the ability of the immune system to build an immune defense against a pathogen (e.g., microorganism, virus, bacterium) upon first exposure to the pathogen, which allows it to later recognize and respond more effectively to the same pathogen upon subsequent exposures. As an alternative to using a whole pathogen to elicit an immune response, a polypeptide from a pathogen can be used (e.g., delivered as a nucleic acid coding sequence) in the vaccination process. The polypeptide is defined as an immunogenic polypeptide because it is capable of eliciting an immune response. For example, an immunogenic polypeptide can comprise a toxoid (defined herein as an inactivated bacterial toxin) or one or more viral subunits or subvirion products. Further, an immunogenic polypeptide can comprise a protein (e.g., an antigen) specific for or associated with a malignancy (e.g., melanoma, breast cancer, and cervical cancer).

AAV11 vectors can be used to deliver a nucleic acid encoding an immunogenic polypeptide from virtually any pathogen (e.g., viruses, bacteria), a portion of a pathogen, or an antigenic polypeptide to elicit an immune response to a pathogen in a subject. For example, a subject can be vaccinated against essentially any virus (e.g., those provided at viralzone.expasy.org on the World Wide Web) or essentially any bacteria (e.g., those provided at globalrph.com/bacteria/on the World Wide Web).

Simply by way of example, a subject can be vaccinated against a virus such as, without limitation, Adenoviruses, Arenaviruses, Arteriviruses, Birnaviruses, Bunyaviruses (e.g., hantavirus), Caliciviruses (e.g., hepatitis E virus, noroviruses), Coronaviruses (e.g., human coronaviruses including SARS-COV-1, SARS-COV-2, and MERS), Filoviruses (e.g., Ebola virus), Flaviviruses (e.g., dengue virus, West Nile virus, yellow fever virus, Japanese encephalitis virus, St. Louis encephalitis virus, tick-borne encephalitis virus), Hepadnaviruses (e.g., hepatitis B virus), Herpesviruses (e.g., herpes simplex viruses (e.g., HSV-1 or HSV-2), varicella-zoster virus, cytomegalovirus, human herpesvirus 6, human herpesvirus 7, human herpesvirus 8, Epstein-Barr virus), Human immunodeficiency virus (HIV), Human papilloma virus (HPV) (e.g., HPV-16 or -18), Orthomyxoviruses (e.g., human influenza viruses), Paramyxoviruses (e.g., measles virus, mumps virus, parainfluenza viruses, respiratory syncytial virus, Newcastle disease virus), Picornaviruses (e.g., hepatitis A virus, enteroviruses such as poliovirus, foot and mouth disease virus, rhinoviruses), Parvoviruses, Papovaviruses (e.g., human papillomaviruses, SV40), Pneumoviruses, Poxviruses (e.g., vaccinia), Rhabdoviruses (e.g., rabies virus, vesicular stomatitis viruses), Reoviruses (e.g., rotaviruses), Retroviruses (e.g., HIV 1, HIV 2, HTLV-1, HTLV-2), and Togaviruses (e.g., sindbis virus, rubella virus).

Simply by way of example, a subject can be vaccinated against a bacteria such as, without limitation, a bacterium of a species selected from Acinetobacter spp. (e.g., Acinobacter baumannii), Bacillus spp. (e.g., Bacillus subtilis), Bartonella spp. (e.g., Bartonella henselae), Bordetella spp. (e.g., Bordetella pertussis), Borelia spp. (e.g., Borelia burgdorferi), Brucella spp. (e.g., Brucella melitensis), Camplybacter spp. (e.g., Camplybacter jejuni), Chlamydia spp. (e.g., Chlamydia pneumoiae), Clostridium spp. (e.g., Clostridium botulinum, Clostridium difficile), Corynebacterium spp. (e.g., Corynebacterium amycolatum), Escherichia spp. (e.g., E. coli 0157: H7), Ehrlichia spp. (e.g., Ehrlichia chaffeensis), Enterococcus spp. (e.g., Enterococcus faecalis), Enterococcus spp. (e.g., Enterococcus faecium), Francisella spp. (e.g., Francisella tularensis), Haemophilus spp. (e.g., Haemophilus influenza), Helicobacter spp. (e.g., Helicobacter pylori), Klebsiella spp. (e.g., Klebsiella pneumonia), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium tuberculosis), Mycoplasma spp. (e.g., Mycoplasma pneumonia), Neisseria spp. (e.g., Neisseria gonorrhoeae), Parachlamydia spp. (e.g., Parachlamydia acanthamoebae), Salmonella spp. (e.g., Salmonella enterica), Shigella spp. (e.g., Shigella sonnei), Staphylococcus spp. (e.g., Staphylococcus aureus), Streptococcus spp. (e.g., Streptococcus pneumonia, Streptococcus pyogenes), Vibrio spp. (e.g., Vibrio cholera, Vibrio vulnificus), and Yersinia spp. (e.g., Yersinia pestis).

By way of example, a subject can be vaccinated against a parasite such as, without limitation, a Plasmodium species (e.g., Plasmodium falciparum, Plasmodium malariae, Plasmodium vivax, Plasmodium knowlesi, Plasmodium ovale curtisi, and Plasmodium ovale wallikeri), a Trypanosoma species, a Toxoplasma species, a Leishmania species, or Cryptosporidium species.

Immunogenic polypeptides, also referred to as antigens, are known in the art or can be readily identified by routine experimentation. With respect to a host, immunogenic polypeptides can be exogenous (e.g., allergens, proteins from transplanted tissues and organs, substances on the surface of foreign cells, toxins, and other foreign particles) or endogenous (e.g., antigens presented by cells that have become infected by bacteria or viruses, blood group antigens on the cell surface of erythrocytes (e.g. H antigen on RBCs, A antigens, and B antigens), or histocompatibility leukocyte antigen (HLA)). Simply by way of example, and without being so limited, representative bacterial and viral antigens include aminopeptidases, capsid proteins or portions thereof, cell wall proteins, chaperone proteins (DnaJ, DnaK), envelope proteins, glycoproteins (e.g., Spike, D, E, G, 160), outer membrane proteins (e.g., OmpA), nucleocapsid proteins, or toxins (e.g., enterotoxins).

In addition to originating from bacteria or viruses, an immunogenic polypeptide also can originate from a cancer cell. For example, an immunogenic polypeptide for use in a vaccine as described herein can be NY-ESO-1 (e.g., to vaccinate against bladder cancer); HER2 (e.g., to vaccinate against breast cancer); HPV16 E7 (e.g., to vaccinate against cervical cancer); carcinoembryonic antigen (CEA) (e.g., to vaccinate against colorectal cancer); Alphafetoprotein (AFP) (e.g., to vaccinate against liver, testicle, and ovarian cancer); WT1 (e.g., to vaccinate against leukemia); MART-1, gp100, and/or tyrosinase (e.g., to vaccinate against melanoma); URLC10, VEGFR1 and/or VEGFR2 (e.g., to vaccinate against non-small lung cell cancer (NSCLC)); CA-125 and/or survivin (e.g., to vaccinate against ovarian cancer); MUC1 (e.g., to vaccinate against pancreatic cancer); and/or MUC2 (e.g., to vaccinate against prostate cancer). Other tumor antigens are known in the art.

In some aspects, the disclosure provides an AAV11 vector comprising at least one AAV11 capsid protein and a nucleic acid encoding a transgene operably linked to a promoter. In some embodiments, the transgene encodes an immunogenic polypeptide as described herein.

In some embodiments, the immunogenic polypeptide is from a virus selected from the group consisting of Adenoviruses, Arenaviruses, Arteriviruses, Birnaviruses, Bunyaviruses, Caliciviruses, Coronaviruses, Ebola viruses, Filoviruses, Flaviviruses, Hepadnaviruses, Herpesviruses, Human immunodeficiency virus (HIV), Human papilloma virus (HPV), Influenza viruses, Orthomyxoviruses, Paramyxoviruses, Picornaviruses, Parvoviruses, Papovaviruses, Pneumoviruses, Poxviruses, Rhabdoviruses, Reoviruses, Retroviruses, Rotaviruses, and Togaviruses. In some embodiments, the immunogenic polypeptide is from a microorganism selected from a species selected from Acinetobacter spp., including, for example, Acinetobacter baumannii; Bacillus spp.; Bartonella spp., including, for example, Bartonella henselae; Bordetella spp.; Borelia spp., including, for example, Borelia burgdorferi; Brucella spp., including, for example, Brucella melitensis; Camplybacter spp., including, for example, Camplybacter jejuni; Chlamydia spp., including, for example, Chlamydia pneumoiae; Clostridium spp., including, for example, Clostridium botulinum; Corynebacterium spp., including, for example, Corynebacterium amycolatum; Escherichia spp., including, for example, E. coli 0157: H7; Ehrlichia spp., including, for example, Ehrlichia chaffeensis; Enterococcus spp., including, for example, Enterococcus faecalis or Enterococcus faecium; Francisella spp., including, for example, Francisella tularensis; Haemophilus spp., including, for example, Haemophilus influenza; Helicobacter spp., including, for example, Helicobacter pylori; Klebsiella spp., including, for example, Klebsiella pneumonia; Legionella spp., including, for example, Legionella pneumophila; Leptospira spp., including, for example, Leptospira interrogans; Listeria spp., including, for example, Listeria monocytogenes; Mycobacterium spp., including, for example, Mycobacterium tuberculosis; Mycoplasma spp., including, for example, Mycoplasma pneumonia; Neisseria spp., including, for example, Neisseria gonorrhoeae; Parachlamydia spp.; Salmonella spp., including, for example, Salmonella enterica; Shigella spp., including, for example, Shigella sonnei; Staphylococcus spp., including, for example, Staphylococcus aureus; Streptococcus spp., including, for example, Streptococcus pneumonia or Streptococcus pyogenes; Vibrio spp., including, for example, Vibrio vulnificus; and Yersinia spp., including, for example, Yersinia pestis In some embodiments, the immunogenic polypeptide is from a parasite. Examples of parasites include, but are not limited to, Plasmodium species (e.g., Plasmodium falciparum, Plasmodium malariae, Plasmodium vivax, Plasmodium knowlesi, Plasmodium ovale curtisi, and Plasmodium ovale wallikeri), Trypanosoma species, Toxoplasma species, Leishmania species, and Cryptosporidium species.

In some embodiments, the immunogenic polypeptide comprises a viral antigen. In some embodiments, the viral antigen comprises a coronavirus receptor-binding domain (RBD) or a fragment of a coronavirus RBD. In some embodiments, the viral antigen comprises a coronavirus spike protein or a fragment of a coronavirus spike protein. In some embodiments, the coronavirus is a SARS-COV-2 virus.

In some embodiments, the nucleic acid expresses a codon-optimized, pre-fusion stabilized full length SARS-COV-2 Spike protein (e.g., Wuhan, Beta, or Delta spike protein) under the control of a promoter. In some embodiments, the nucleic acid expresses a subunit (e.g., S1, S2, RBD, furin recognition site) of the full length SARS-COV-2 Spike protein under the control of a promoter. In some embodiments, the promoter is a minimal CMV promoter. In some embodiments, the promoter is an SV40 promoter. In some embodiments, the promoter is a short EFla promoter (EFS). In some embodiments, the promoter is a minimal CMV promoter (miniCMV). In some embodiments, the promoter is a full CMV promoter. In some embodiments, the nucleic acid further comprises an SV40 polyA. In some embodiments, the nucleic acid further comprises a short synthetic polyA (SPA).

In some embodiments, the immunogenic polypeptide comprises a bacterial antigen. In some embodiments, the immunogenic polypeptide comprises a parasitic antigen. In some embodiments, the immunogenic polypeptide comprises a fungal antigen.

In some embodiments, the immunogenic polypeptide comprises a cancer antigen. A number of cancer antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. In some embodiments, the cancer antigen is NY-ESO-1, HER2, HPV16 E7, carcinoembryonic antigen (CEA), MSLN, WT1, MART-1, gp100, tyrosinase, URLC10, VEGFR1, VEGFR2, survivin, MUC-1, or MUC-2. Additional, non-limiting examples of tumor or cancer antigens include, but are not limited to, prostate stem cell antigen (PSCA), PSMA (prostate-specific membrane antigen), β-catenin-m, B cell maturation antigen (BCMA), alpha-fetoprotein (AFP), cancer antigen-125 (CA-125), CA19-9, calretinin, epithelial membrane protein (EMA), epithelial tumor antigen (ETA), Mammaglobin-A, melanoma-associated antigen (MAGE), CD34, CD45, CD99, CD117, chromogranin, cytokeratin, desmin, glial fibrillary acidic protein (GFAP), gross cystic disease fluid protein (GCDFP-15), EBV, HMB-45 antigen, protein melan-A (melanoma antigen recognized by T lymphocytes; MART-1), livin, myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD19, CD22, CD27, CD30, CD70, GD2 (ganglioside G2), EphA2, CSPG4, CD138, FAP (Fibroblast Activation Protein), CD171, kappa, lambda, 5T4, .alpha.sub.v.beta.sub.6 integrin, B7-H3, B7-H6, CAIX, CD19, CD20, CD22, CD30, CD33, CD44, CD44v6, CD44v7/8, CD70, CD123, EGFR, EGP2, EGP40, EpCAM, fetal AchR, FR.alpha., GAGE, GD3, HLA-A1+MAGE1, MAGE-3, HLA-A1+NY-ESO-1, IL-11R.alpha., IL-13R.alpha.2, Lewis-Y, Muc16, NCAM, NKG2D Ligands, PRAME, ROR1, SSX, TAG72, TEMs, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), HSP70-2/m, and HLA-A2-R170J, an abnormal ras protein, or an abnormal p53 protein.

### Other Regulatory Elements of the Nuclei Acid

In some embodiments, a nucleic acid described herein further comprises other nucleic acid elements. In some embodiments, a nucleic acid described herein further comprises other regulatory elements. Examples of regulatory elements include, but are not limited to, promoters, enhancers, introns, silencers, insulators, tethering elements and post-transcriptional regulatory elements (e.g., tripartite posttranscriptional regulatory element, hepatitis virus posttranscriptional regulatory element, hepatitis B virus posttranscriptional regulatory element, Woodchuck hepatitis virus posttranscriptional regulatory element). In some embodiments, the other regulatory elements comprise a Woodchuck Hepatitis Virus (WHP) Posttranscriptional Regulatory Element (WPRE). In some embodiments, the nucleic acid further comprises a polyadenylation (polyA) sequence. In some embodiments, the polyA sequence is a short synthetic polyA (SPA) sequence. In some embodiments, the nucleic acid is minimally flanked by Inverted Terminal Repeats (ITRs).

In some embodiments, the nucleic acid further comprises an intron. In some embodiments, the intron is located downstream of the promoter. In some embodiments, the promoter is a CMV promoter and the nucleic acid further comprises an intron located downstream of the promoter.

### Methods of Eliciting an Immune Response in a Subject

An AAV11 vector comprising a nucleic acid (e.g., a transgene) encoding an antigenic portion of a pathogen or tumor tissue can be used to immunize (e.g., vaccinate, elicit a protective immune response to the pathogen) subjects against infection or disease, i.e., to elicit a protective immune response that reduces the risk of the subjects developing the infection, or reduces the risk of the subject developing a severe infection or triggers the reduction and possible elimination of tumor tissue. Such a vaccine can be prepared as a vaccine composition, e.g., suspended in a physiologically compatible carrier and administered to a subject (e.g., a human, a NHP, a rodent, a companion animal, an exotic animal, and livestock). Suitable carriers include saline, which may be formulated with a variety of buffering solutions (e.g., phosphate buffered saline), lactose, sucrose, calcium phosphate, gelatin, dextran, agar, pectin, and water.

In some aspects, the disclosure provides a composition comprising an AAV11 vector as described herein, and a pharmaceutically acceptable carrier. In some aspects, the disclosure provides a vaccine comprising an AAV11 vector as described herein.

A vaccine composition can include one or more adjuvants. Some adjuvants contain a substance designed to protect the antigen from rapid catabolismsuch as aluminum hydroxide or mineral oil, and a specific or nonspecific stimulator of immune responses, such as lipid A or Bortadella pertussis. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A, quil A. SBAS1c, SBAS2 (Ling et al., 1997. Vaccine 15:1562-1567), SBAS7, Al(OH)3 and CpG oligonucleotide (WO 96/02555).

In some embodiments of the vaccines described herein, the adjuvant may induce a Th1 type immune response. Suitable adjuvant systems can include, for example, a combination of monophosphoryl lipid A, preferably 3-de-O-acylated monophosphoryl lipid A (3D-MPL) together with an aluminum salt. An enhanced system involves the combination of a monophosphoryl lipid A and a saponin derivative, particularly the combination of 3D-MLP and the saponin QS21 as disclosed in WO 94/00153, or a less reactogenic composition where the QS21 is quenched with cholesterol as disclosed in WO 96/33739. Previous experiments have demonstrated a clear synergistic effect of combinations of 3D-MLP and QS21 in the induction of both humoral and Th1 type cellular immune responses. A particularly potent adjuvant formation involving QS21, 3D-MLP and tocopherol in an oil-in-water emulsion is described in WO 95/17210 and can be included in a vaccine composition as described herein.

In some aspects, the disclosure provides a method of eliciting an immune response in a subject comprising administering to the subject a composition as described herein. In some aspects, the disclosure provides a method of treating or preventing a disease in a subject comprising administering to the subject a composition as described herein.

A vaccine composition typically is administered in sufficient amounts to transduce or infect the host cells and to provide sufficient levels of expression to provide an immunogenic benefit without undue adverse effects. In some embodiments, the composition is administered to the subject only once. In some embodiments, the composition is administered to the subject more than once. In some embodiments, the composition is administered to the subject twice. In In some embodiments, the time between the administration of two consecutive doses is at least one month. some embodiments, the time between the administration of two consecutive doses is at least two months. In some embodiments, the time between the administration of two consecutive doses is at least three months. In some embodiments, the time between the administration of two consecutive doses is at least four months. In some embodiments, the time between the administration of two consecutive doses is at least five months. In some embodiments, the time between the administration of two consecutive doses is at least six months. In some embodiments, the time between the administration of two consecutive doses is at least seven months. In some embodiments, the time between the administration of two consecutive doses is at least eight months. In some embodiments, the time between the administration of two consecutive doses is at least nine months. In some embodiments, the time between the administration of two consecutive doses is at least ten months. In some embodiments, the time between the administration of two consecutive doses is at least eleven months. In some embodiments, the time between the administration of two consecutive doses is at least one year. In some embodiments, the time between the administration of two consecutive doses is at least two years.

The dose of a vaccine composition as described herein (e.g., an AAV11 capsid and a nucleic acid that encodes an immunogenic polypeptide) that can be administered to a subject will depend primarily on factors such as the condition being treated, and the age, weight, and health of the subject. For example, a therapeutically effective dosage of a vaccine composition as described herein for administration to a human subject generally is in the range of from about 0.1 ml to about 10 ml (e.g., about 0.1 ml, about 0.2 ml, about 0.3 ml, about 0.4 ml, about 0.5 ml, about 0.6 ml, about 0.7 ml, about 0.8 ml, about 0.9 ml, about 1 ml, about 2 ml, about 3 ml, about 4 ml, about 5 ml, about 6 ml, about 7 ml, about 8 ml, about 9 ml, about 10 ml) of a solution containing a viral dosage from about 1×10⁷ to about 1×10¹⁶ genome copies (GCs) of a virus. In some embodiments, the composition administered to the subject comprises a viral dosage of at least 1×10⁷ genome copies, at least 5×10⁷ genome copies, at least 1×10⁸ genome copies, at least 5×10⁸ genome copies, at least 1×10⁹ genome copies, at least 5×10⁹ genome copies, at least 1×10¹⁰ genome copies, at least 5×10¹⁰ genome copies, at least 1×10¹¹ genome copies, at least 5×10¹¹ genome copies, at least 1×10¹² genome copies, at least 5×10¹² genome copies, at least 1×10¹³ genome copies, at least 5×10¹³ genome copies, at least 1×10¹⁴ genome copies, at least 5×10¹⁴ genome copies, at least 1×10¹⁵ genome copies, at least 5×10¹⁵ genome copies, or at least 1×10¹⁶ genome copies. In some embodiments, the composition administered to the subject comprises a viral dosage of 10⁷ to 10⁸ genome copies, 10⁸ to 10⁹ genome copies, 10⁹ to 10¹⁰ genome copies, 10¹⁰ to 10¹¹ genome copies, 10¹¹ to 10¹² genome copies, 10¹² to 10¹³ genome copies, 10¹³ to 10¹⁴ genome copies, 10¹⁴ to 10¹⁵ genome copies, 10¹⁵ to 10¹⁶ genome copies, 10⁸ to 10¹⁰ genome copies, 10¹⁰ to 10¹² genome copies, 10¹² to 10¹⁴ genome copies, 10¹⁴ to 10¹⁶ genome copies, 10⁷ to 10⁹ genome copies, 10⁹ to 10¹¹ genome copies, 10¹¹ to 10¹³ genome copies, 10¹³ to 10¹⁵ genome copies, 10⁷ to 10¹⁰ genome copies, 10¹⁰ to 10¹³ genome copies, 10³ to 10⁸ genome copies, 10⁸ to 10¹³ genome copies, or 10¹³ to 10¹⁶ genome copies. In some cases, for example, a suitable dose may include administering a higher amount of viral vectors over a short time (e.g., hours, days, or weeks); in some cases, for example, a suitable dose may include administering a lower amount of viral vectors for a prolonged period of time (e.g., weeks, months, years).

Conventional and pharmaceutically acceptable routes of administration include, but are not limited to, intramuscular injection. Additional routes of administration include, for example, orally, intranasally, intratracheally, by inhalation, intravenously, subcutaneously, sublingual, intradermally, intravaginally, rectal, or transmucosally. In some instances, a vaccine composition in liquid form can be delivered nasopharyngeally via, e.g., a spray or an inhaler, or via a capsule or tab, where the liquid is released upon entering the gut and the AAV11-based vaccine is taken up by gut-associated lymphoid tissue.

The present methods can include administration of a prophylactically effective amount of a vaccine composition as described herein to a subject in need thereof, e.g., a subject who is at risk of developing a disease or an infection. In some embodiments, the subject is a mammal. In some embodiments, the subject is selected from the group consisting of a human, a non-human primate, a rodent (e.g., a mouse, a rat, a hamster), an exotic animal, a companion animal, and livestock. In some embodiments, the subject has not yet been, but will likely be, exposed to such a disease or to a microorganism causing such an infection. In addition, some infections linger (e.g., COVID “long-haulers”) or flare-up periodically, and a suitable dose of a vaccine composition as described herein can be used to treat such infections. In some embodiments, the subject is at risk of developing an infection or cancer. In some embodiments, the subject is at risk of developing a bacterial infection. In some embodiments, the subject is at risk of developing a viral infection. In some embodiments, the subject is at risk of developing a disease selected from the group consisting of SARS-COV-1 and SARS-COV-2 (COVID-19).

### Methods of Making an AAV11-Based Vaccine

AAV11-based vaccine compositions as described herein (e.g., AAV11 vectors comprising a nucleic acid encoding an immunogenic polypeptide) can be produced using any number of methods known in the art. For example, AAV11 vectors comprising a nucleic acid can be produced in insect cells or mammalian cells.

Stable engineered cell lines for producing AAV11 vectors can be generated by introducing replication (Rep) and structural capsid (Cap) genes or an appropriate rAAV genome to produce packaging or producer cell lines, respectively. AAV11 vectors can be produced from packaging cell lines following transfection of the AAV11 capsid sequences and the co-infection with a helper virus, such as adenovirus (Ad) or Herpes Simplex Virus (HSV) or via a single infection with a recombinant helper viral vector containing a rAAV genome. For producer cell lines, AAV11 vectors can be generated following a single-step infection with an Ad or HSV helper virus. Stable cell lines have been reported to produce relatively high AAV vector genome (vg) particles per cell (up to 10,000 vg per producing cell). Packaging and producer cell lines have been generated using cell lines capable of both adherent and suspension growth, allowing for processes to be developed that utilize traditional tissue culture systems for small scale, combined with larger-scale manufacturing performed in suspension bioreactors.

AAV11 vectors also can be produced using a Baculovirus (BV) expression system, which is able to produce complex glycosylated recombinant proteins at high levels and at high cell densities. The BV system was developed to produce viral particles (e.g., vectors) without the need to co-infect with a helper virus, and has evolved to the use of a two-vector approach or large-scale production that involves AAV-infected Baculovirus cells that separately carry each of the required AAV components (rAAV genome, Rep, and Cap genes), which can be used to drive a sustained production phase.

Transient transfection of plasmid DNA into mammalian cells for the production of AAV11 vectors is another strategy commonly used in clinical grade manufacturing. AAV11 vectors can be produced in human embryonic kidney 293 cells (HEK293) or variants thereof by introducing DNA plasmids that carry the replication (Rep) and AAV11 structural capsid (Cap) genes, the transgene to be carried by the AAV11 vector, and the specific genes that provide helper Ad function. The cells that are successfully transfected with all the necessary plasmids will produce AAV11 vectors comprising the transgene encoding the immunogenic polypeptide.

In some aspects, the disclosure provides a method of manufacturing an AAV11 vector, comprising (i) transfecting a host cell expressing an AAV11 capsid protein with a nucleic acid encoding an immunogenic polypeptide as described herein operably linked to a promoter, and (ii) culturing the host cell under conditions in which AAV11 vectors comprising at least one AAV11 viral capsid protein as described herein comprising the nucleic acid are produced. In some embodiments, the host cell is a producer cell. In some embodiments, the host cell is a packaging cell.

In some aspects, the disclosure provides a method of manufacturing an AAV11 vector, comprising (i) transfecting a producer cell with a nucleic encoding an AAV11 capsid protein and another nucleic acid encoding an immunogenic polypeptide operably linked to a promoter, and (ii) culturing the producer cell under conditions in which AAV11 vectors comprising at least one AAV11 viral capsid protein as described herein comprising the nucleic acid are produced.

In some embodiments, the antigen plasmid further comprises a Woodchuck Hepatitis Virus (WHP) Posttranscriptional Regulatory Element (WPRE). In some embodiments, the viral antigen comprises a coronavirus spike protein, or a fragment of a coronavirus spike protein. In some embodiments, the viral antigen comprises a coronavirus receptor-binding domain (RBD), or a fragment of a coronavirus RBD. In some embodiments, the coronavirus is a SARS-COV-2 virus.

In some embodiments, the promoter is selected from the group consisting of a CAG promoter, an EF1 alpha promoter, a p5 promoter, a p19 promoter, a p40 promoter, a SV40 promoter, an elongation factor short (EFS) promoter, a cytomegalovirus (CMV) promoter, and a minimal CMV (mini-CMV) promoter.

In some embodiments, the antigen plasmid comprises a nucleic acid sequence at least 90% identical to any one of SEQ ID NOs: 3-6. In some embodiments, the antigen plasmid comprises a nucleic acid sequence that is any one of SEQ ID NOs: 3-6.

In some embodiments, the cell is an insect cell. In some embodiments, the insect cell is a baculovirus cell. In some embodiments, the cell is a mammalian cell.

In some embodiments, the nucleic acid encoding an immunogenic polypeptide is expressed from a recombinant AAV genome.

As used herein, nucleic acids can include DNA and RNA, and include nucleic acids that contain one or more nucleotide analogs or backbone modifications. A nucleic acid can be single stranded or double stranded, which usually depends upon its intended use. An AAV11 VP1 capsid protein can have the amino acid sequence shown in SEQ ID NO: 1, which can be encoded by the nucleic acid sequence shown in SEQ ID NO: 2. An AAV11 capsid protein also can have a sequence that differs from SEQ ID NO: 1 or 2. For example, nucleic acids and polypeptides that differ in sequence from SEQ ID NO: 1 or 2 (or from the promoter sequences disclosed in SEQ ID NOs: 10-15) can have at least 80% sequence identity (e.g., at least 85%, 90%, 95%, or 99% sequence identity) to SEQ ID NO: 1 or 2 (or to the promoter sequences disclosed in SEQ ID NOs: 10-15).

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

It would be appreciated that changes can be introduced into a nucleic acid molecule, leading to changes in the amino acid sequence of the encoded polypeptides. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3): 345-352), which provides frequency tables for amino acid substitutions), and a non-conservative substitution is one in which an amino acid residue is replaced with an amino acid residue that does not have a similar side chain.

It would be appreciated by a skilled artisan that the AAV capsid protein contains highly structurally conserved beta-barrel motifs, which maintain the icosahedral architecture of the viral capsid, as well as hypervariable regions between the beta motifs, which fall within loops on the surface of the viral capsid. It also would be appreciated by a skilled artisan that one or more changes in the sequence of the AAV11 capsid protein can be at a position that falls within the beta-barrel motifs, provided an icosahedral architecture of the viral capsid is maintained, within the hypervariable regions, or within both. In addition, given the high sequence identity between AAV11 and rh32.33, it would be understood that, in some instances, the R167 and S259 positions within the AAV11 capsid sequence (SEQ ID NO: 1) are fixed (i.e., remain as arginine and serine, respectively), irrespective of sequence changes outside of those positions. Further, it would be understood that, in some instances, the R167 position and/or the S259 position within the AAV11 capsid sequence (SEQ ID NO: 1) are changed to a different amino acid (e.g., any amino acid other than arginine and serine, respectively). In some instances of this latter embodiment, however, changes in the AAV11 capsid proteins (SEQ ID NO: 1) are not R167K and S259N. Lastly, it would be understood by a skilled artisan that the AAV11 VP1 nucleic acid sequence (SEQ ID NO: 2) could be changed to encode the corresponding changes in the AAV11 VP1 capsid sequence discussed herein.

Nucleic acids can be obtained or produced using any number of methods including, without limitation, chemical synthesis, recombinant nucleic acid technology, and/or the polymerase chain reaction (PCR). General PCR techniques are described, for example in PCR Primer: A Laboratory Manual (Dieffenbach & Dveksler, Eds., 1995, Cold Spring Harbor Laboratory Press), and recombinant nucleic acid techniques are described, for example, in Molecular Cloning; A Laboratory Manual (Sambrook et al., 1989, Cold Spring Harbor Laboratory Press, Cold Spring Harbor, NY).

Constructs containing nucleic acid molecules that encode polypeptides are also provided. Constructs, including expression constructs, are commercially available or can be produced by recombinant technology. A construct containing a nucleic acid molecule can have one or more elements for expression operably linked to such a nucleic acid molecule, and further can include sequences such as those encoding a selectable marker (e.g., an antibiotic resistance gene), and/or those that can be used in purification of a polypeptide (e.g., 6×His tag). Elements for expression include nucleic acid sequences that direct and regulate expression of nucleic acid coding sequences. One example of an expression element is a promoter sequence (e.g., CaMV 35S, CMV, SV40, EF-1 alpha, and TEF1). Expression elements also can include one or more introns, enhancer sequences, response elements, or inducible elements that modulate expression of a nucleic acid molecule. Expression elements can be of bacterial, yeast, insect, mammalian, or viral origin and vectors can contain a combination of expression elements from different origins. As used herein, operably linked means that elements for expression are positioned in a vector relative to a coding sequence in such a way as to direct or regulate expression of the coding sequence.

A nucleic acid molecule, e.g., a nucleic acid molecule in a construct (e.g., an expression construct) can be introduced into a host cell. The term “host cell” refers not only to the particular cell(s) into which the nucleic acid molecule has been introduced, but also to the progeny or potential progeny of such a cell. Many suitable host cells are known to those skilled in the art; host cells can be prokaryotic cells (e.g., E. coli) or eukaryotic cells (e.g., yeast cells, insect cells, plant cells, mammalian cells). Representative host cells can include, without limitation, A549, WEHI, 3T3, 10T1/2, BHK, MDCK, COS 1, COS 7, BSC 1, BSC 40, BMT 10, VERO, WI38, HeLa, 293 cells, Saos, C2C12, L cells, HT1080, HepG2 and primary fibroblast, baculovirus cells, hepatocyte, and myoblast cells derived from mammals including human, monkey, mouse, rat, rabbit, and hamster. Methods for introducing nucleic acid molecules into host cells are well known in the art and include, without limitation, calcium phosphate precipitation, electroporation, heat shock, lipofection, microinjection, and viral-mediated nucleic acid transfer (e.g., transduction). In some embodiments, the host cell is a producer cell. In some embodiments, the host cell is a packaging cell.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1: Durability and Protection of a Single-Dose Optimized AAV-Based COVID-19 Vaccine in Nonhuman Primate

### Introduction

The COVID19 pandemic is still threatening the health of citizens around the world and its nations' economies. The approved vaccines have shown excellent safety and efficacy to prevent COVID-19, the disease caused by the SARS-COV-2 strain (1-4). As vaccination campaigns advanced, they dramatically reduced the risk of serious disease and death in the vaccinated, however over time a reduction in effectiveness was reported.

As the pandemic progressed globally, the SARS-COV-2 genome mutated allowing for variations conferring neutralization escape as well as increase in infectivity. D614G was one of the first mutation to become globally prevalent and was found to be associated with increased viral load in the upper respiratory tract but not neutralization escape from antibodies generated against the parental Wuhan strain (5-7). In December 2020 and January 2021, several neutralization escape variants of SARS-COV-2 emerged in different locations with distinct mutations in the genome, most notably in the N-terminal domain (NTD), receptor binding domain (RBD) and near the furin cleavage site of the Spike protein being used in several vaccine development projects (8-12). The WHO classified these as variants of concern or VOC, variants of interest or VOI and variants under monitoring (VUM or VBM) (https://www.who.int/en/activities/tracking-SARS-COV-2-variants/). The cross-reactivity of antibodies elicited by natural infection with the Wuhan parental strain or by vaccination with the approved Wuhan Spike-based vaccines has been shown to be less potent against some VOC (13-18). The Beta variant was one of the better escape variants (14, 19), although potent antibody responses against Wuhan also show likely protective immunity against Beta (20, 21). Many breakthrough infections have also been reported recently caused by Delta VOC (22, 23). The Omicron variant emerged and was sequenced for the first time in South Africa. The Spike protein of Omicron presents more than 30 mutations compared to the ancestral Wuhan Spike and data suggests that Omicron might be one of the best escape variant emerged to date (biorxiv.org/content/10.1101/2021.12.14.472630v1.full.pdf, medrxiv.org/content/10.1101/2021.12.14.21267755v1.full.pdf).

Second, immunity elicited by natural infection or vaccination appears to wane in some cases. Antibody levels elicited by the mRNA-based vaccines for example, which are the most commonly used in the US and Europe, appear to progressively wane after 2 doses of immunization by as much as 10-fold in 6 months (24-26). Other vaccines, such as the single shot Ad26, appears to perhaps provide more durable immunity, but overall demonstrates lower protection from disease and reduced antibody levels compared to mRNA at its peak efficacy (27).

The emerging variants of concern and the waning immunity in the vaccinated have prompted manufacturers and health authorities to recommend the need of a third dose as a booster. While mRNA manufacturers have developed and performed initial clinical studies on VOC-based COVID vaccines, immunity with VOC-adapted vaccine candidates is only modestly superior to boosting with the original Wuhan-strain based vaccine. To avoid extensive studies and timelines that authorization of a new vaccine candidate, the already approved Wuhan-based mRNA vaccines have been deployed as booster.

The path to approval of second-generation vaccines has been complex in light of the approval of safe and highly effective vaccines. The pre-clinical efficacy of an AAV-based COVID19 vaccine (AAVCOVID) was previously reported (28). AAVCOVID candidates showed durable and protective immunity in non-human primate (NHP) models upon immunization with a single dose. Neutralizing antibody titers were maintained at peak levels for at least 1-year post-immunization (and continue to hold levels at 16 months). AAVCOVID leveraged established manufacturing capacity in the industry, which can be scaled. Lastly, studies indicated the vaccine product was stable for 1 month at room-temperature.

The goal of this work was to optimize the AAVCOVID to reduce the dose and develop a VOC-specific vaccine on the platform, in part to establish the robustness of the findings across multiple antigens. Reported herein is protection data of the previously described AAVCOVID vaccine candidates at a lower dose in a macaque challenge model. Additionally, AAVCOVID vectors were engineered and their potency improved by 10-40-fold in mouse and NHP. The most potent vaccine to the Beta VOC was also adapted, showing a fast and efficient adaptability of the platform. Finally, it is demonstrated that the optimized AAVCOVID candidates can confer protection against VOC at lower doses.

### Results

### AAVCOVID Vaccines Elicit Durable Immunogenicity in Rhesus Macaques

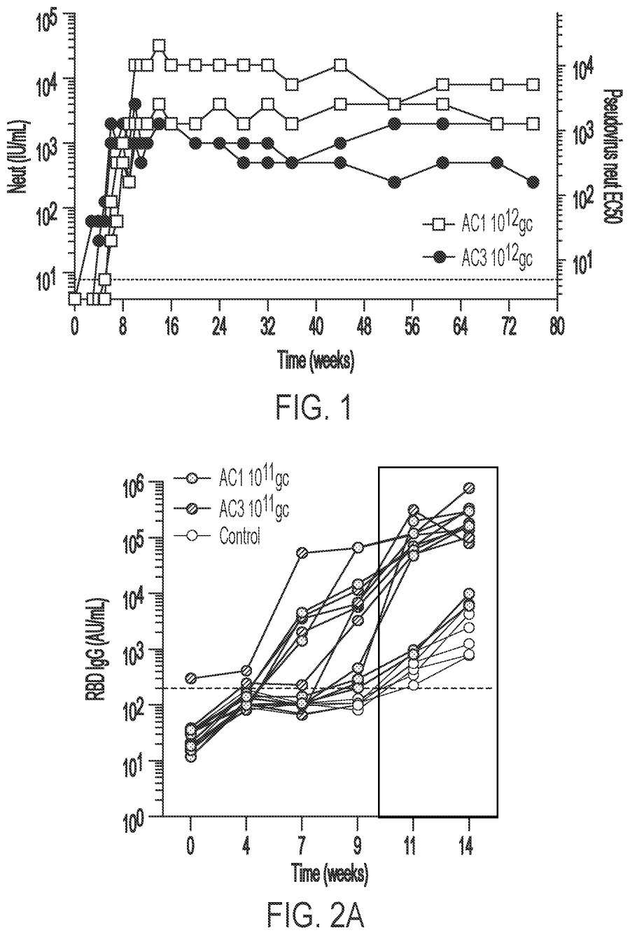

AC1 and AC3 vaccines have been characterized in mouse models (28). Briefly, AC1 expresses the full-length prefusion stabilized Wuhan Spike (Spp) under the control of an SV40 promoter and AC3 the secreted S1 subunit of Wuhan Spike under the control of a CMV promoter, and both are AAVrh32.33 capsid based. Both candidates at high dose elicited durable, up to 11 months, neutralizing antibody responses in rhesus macaques (n=2/candidate) (28). FIG. 1 shows that the antibody response remains stable and at peak levels 18 months (week 70) after a single dose administration.

### Low Doses of First Generation AAVCOVID Only Partially Protect Cynomolgus Macaques

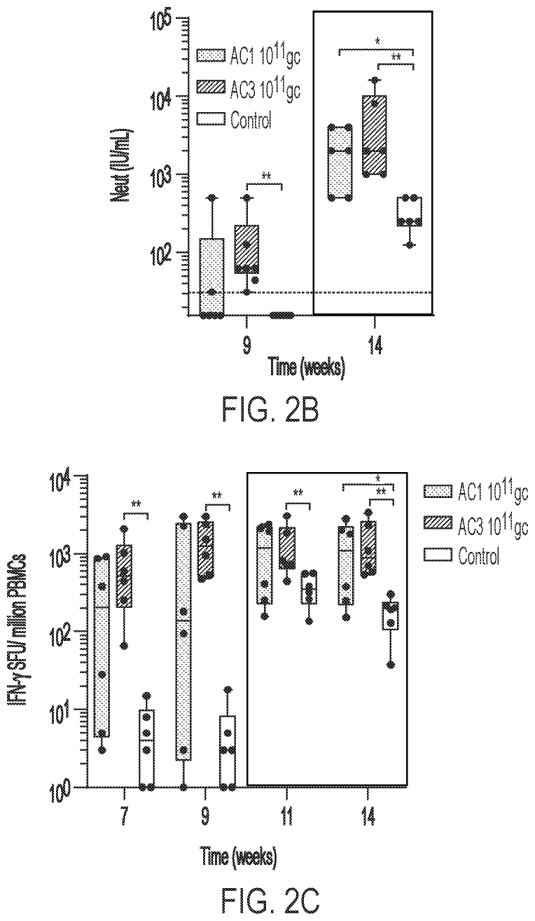

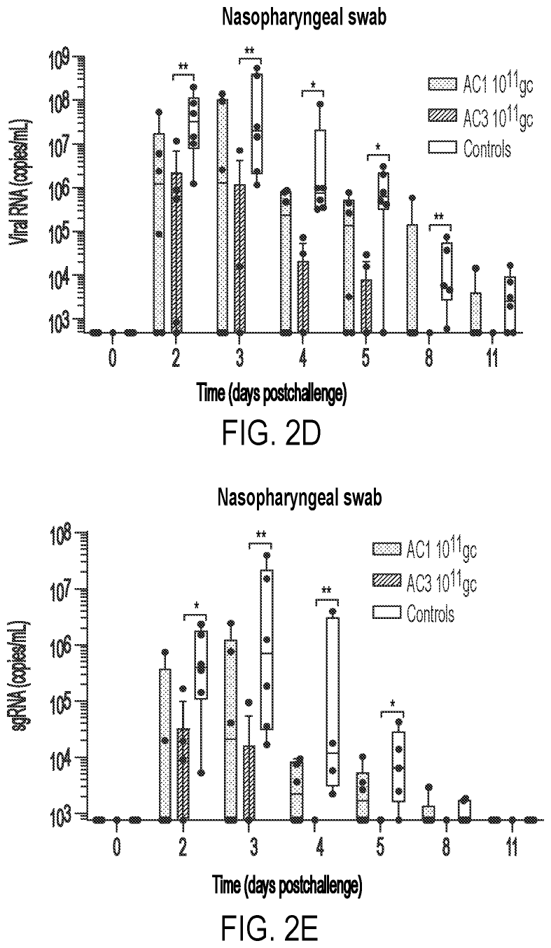

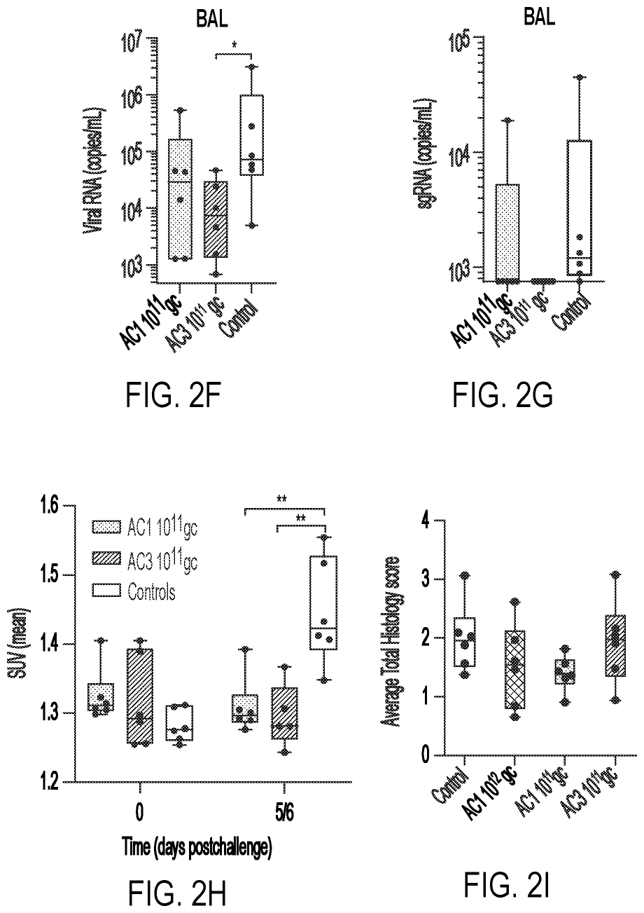

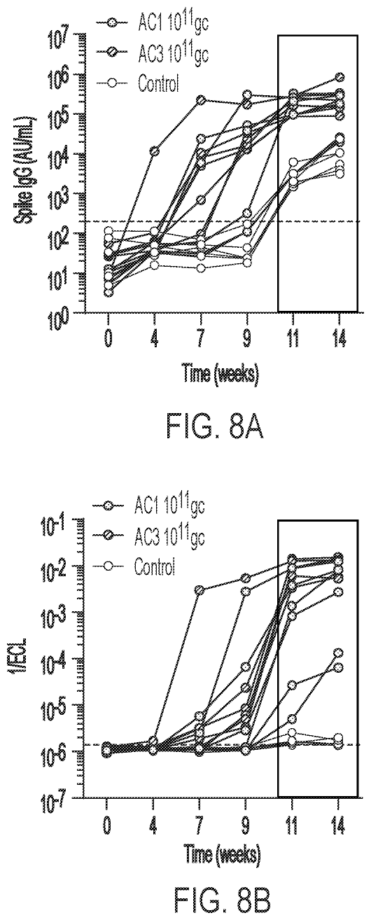

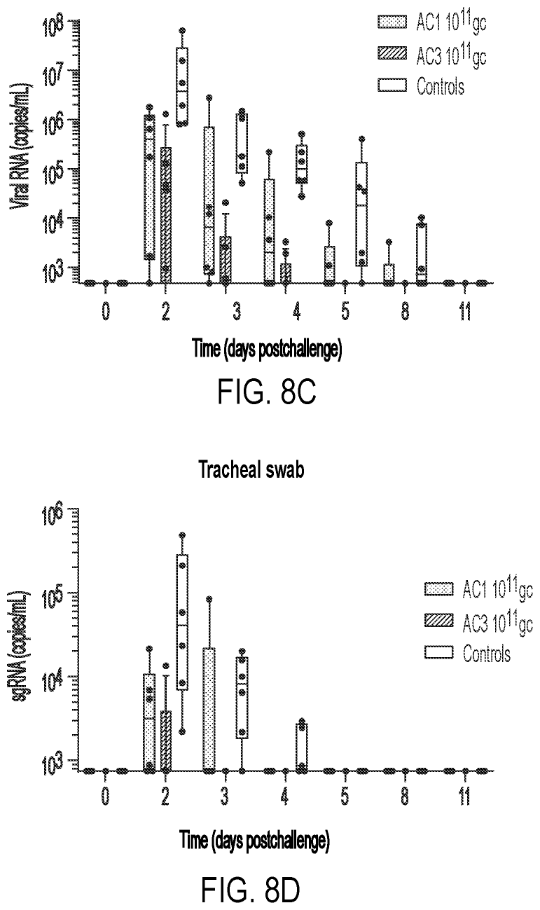

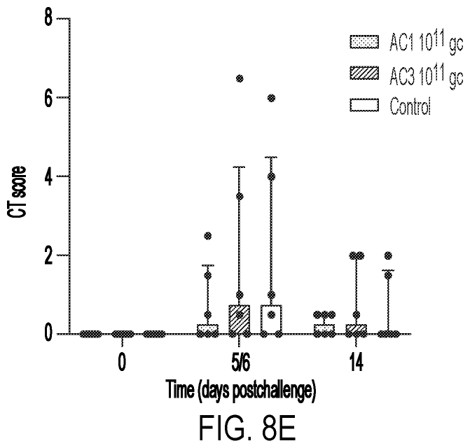

AC1 at a dose of 10¹² gc confers near-sterilizing immunity against SARS-COV-2 challenge in NHPs (28). It was assessed if lower doses of these candidates would be able to provide protective immunity upon a single dose administration. Cynomolgus macaques (n=6/group) were vaccinated with 1011 gc total of AC1 or AC3 vaccine candidates, and a third group was not vaccinated as a control. Antibody and T cell responses were followed for 9 weeks. All animals vaccinated with AC3 showed seroconversion of Wuhan RBD-binding and neutralizing antibodies by week 9 (FIGS. 2A, 2B, 8A and 8B). AC1, however, failed to seroconvert all animals (FIG. 2A) and neutralizing antibody titers were below the detection limits in most of them (FIG. 2B). Same trends were observed in IFN-γ ELISPOT (FIG. 2C).

All the animals were challenged with 10⁵ pfu of SARS-COV-2 (BetaCoV/France/IDF/0372/2020) (29) on week 9.5 after vaccination. This variant presents the differential V367F mutation compared to the B.1 ancestral strain. Vaccinated groups were partially protected from infection in the upper respiratory tract (FIGS. 2D and 2E). 3 of 6 animals in the AC1 and AC3 groups presented detectable viral load (viral RNA and subgenomic RNA) in the nasal swabs, although the virus was cleared faster in the AC3 animals than in the controls (area under the curve (AUC) significantly smaller than controls), while the unprotected AC1 animals showed the same trend as controls (AUC statistically not different compared to controls). The remaining 3 animals in each group presented no viral load in the nasal swab, except for one animal in the AC1 group with a breakthrough in viral RNA on day 2. Similar observations were made in tracheal swabs (FIGS. 8C and 8D). Bronchoalveolar lavage (BAL) was also analyzed to assess protection of the lower respiratory tract. AC1 and AC3 cohorts showed trends to lower viral RNA in the lungs, although detectable, while subgenomic RNA was undetectable in all except one AC1 NHP (FIGS. 2F and 2G). This observation was confirmed by the analysis of lung lymph nodes by PET scan (FIG. 2H). Vaccinated animals did not show an activation of lymph nodes after challenge, which was observed in control animals, due to an active SARS-COV-2 infection in the lungs (FIG. 2H). CT scan did not reveal a significant difference in lung lesions due to the mild phenotype of SARS-COV-2 infection in NHPs (FIG. 8E). Lung histology analysis of vaccinated animals 30 to 35 days after challenge suggests less lesions due to COVID19 infection in AC1 vaccinated animals while no significant difference was observed between the scores of controls and AC3 vaccinated animals (FIG. 2I).

Antibody responses after challenge increased in all the animals, including controls (FIGS. 2A, 2B, 8A and 8B). FIG. 2A clearly shows that 2 of the animals treated with AC1 were non-responders, since the antibody levels after challenge followed the same trend as the unvaccinated and challenged controls. All AC3 animals however did seroconvert prior to the challenge, indicating that at the 10¹¹ gc level the AAVCOVID platform can perform reliably.

### Second Generation AAVCOVID Platform is Optimized for Capsid and Promoter

Given that lower doses of AC1 and AC3 candidates failed to provide complete protection against SARS-COV-2 challenge in NHPs at lower doses, the AAVCOVID vaccine platform to altered to increase the potency of the vaccine candidates. First, the capsid platform was changed from AAVrh32.33 to AAV11 serotype. AAV11 was first isolated from the liver of a cynomolgus monkey (30). From structural comparison with other known AAV serotypes, AAVrh32.33, AAV4 and AAV12 are the closest related serotypes to AAV11 (31). The VP1 sequence of AAV11 and AAVrh32.33 are 99.7% homologous with 2 amino acid difference (K167R and T259S in AAV11). AAV11 vectors containing the same cassette as AC1 (SV40 promoter expressing Spp) were produced and tested in mouse immunogenicity studies. As a result of the homology between AAVrh32.33 and AAV11, the pro-inflammatory nature of the capsid and low seroprevalence of neutralizing antibodies against the AAVrh32.33 was expected to remain an advantage for AAV11.

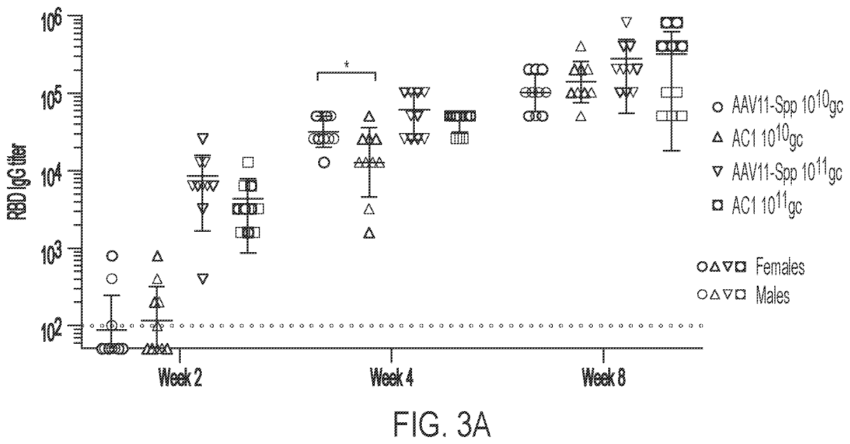

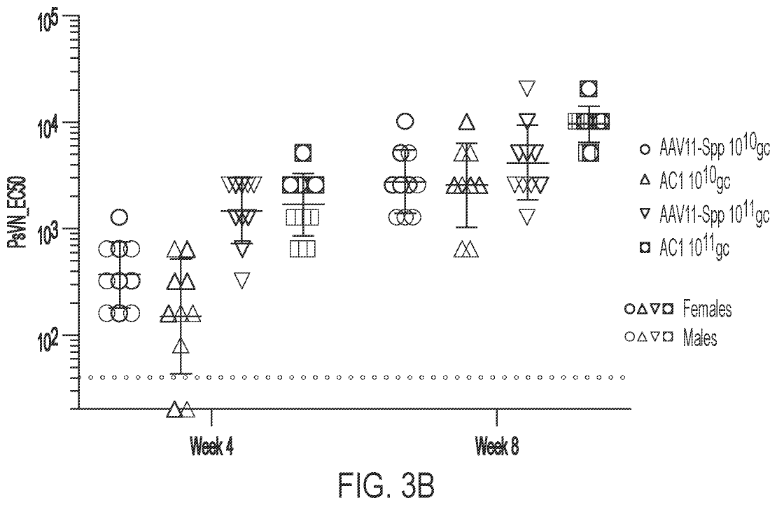

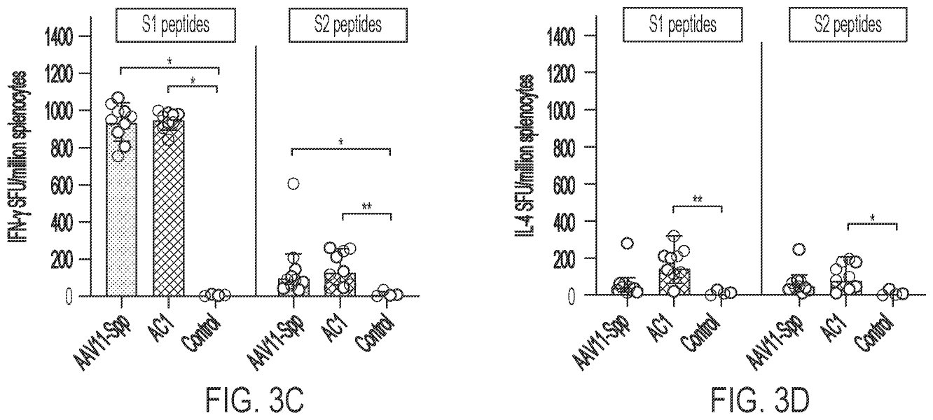

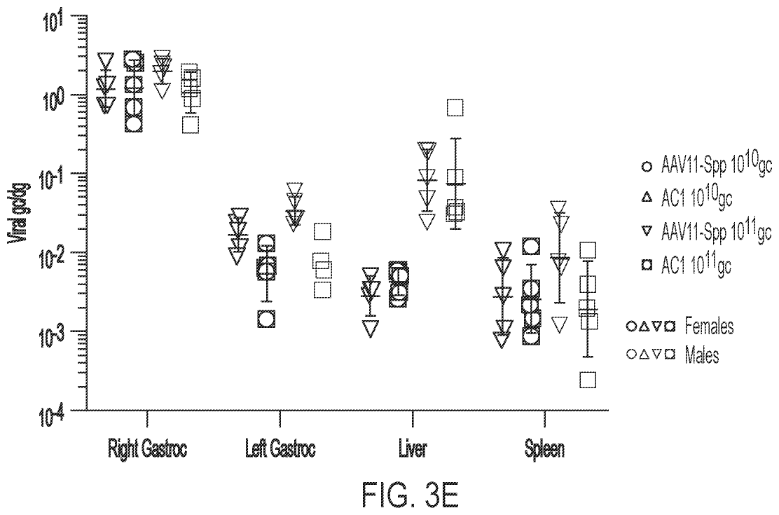

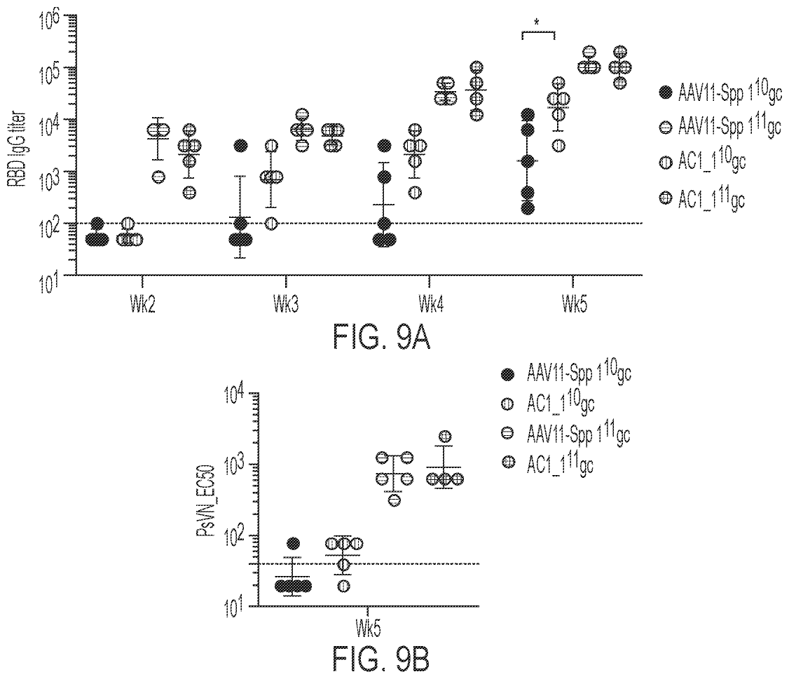

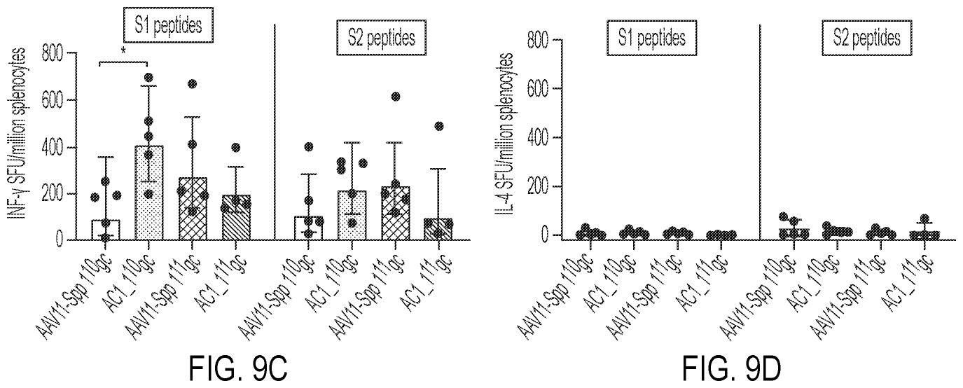

To study the immunogenicity of AAV11-based AAVCOVID vaccines, 6-8 weeks male and female C57BL/6 mice were injected with 10¹¹ and 10¹⁰ gc dose of AAV11-Spp vaccine. As a control, the AA Vrh32.33-based AC1 candidate was also injected. Spike binding and neutralizing responses were similar between mice vaccinated with AC1 and AAV11-Spp across doses and genders (FIGS. 3A and 3B). Cellular responses to the transgene were also preserved for the AAV11-based candidate, with robust IFN-γ responses against Spike peptides, mainly subunit 1 (S1) peptides and very low IL-4 secretion (FIGS. 3C and 3D). The biodistribution pattern of the vectors was analyzed on day 7 after IM administration, same distribution profiles were observed for AAVrh32.33 and AAV11 with most vector copies in the injected muscle (right gastrocnemius) (FIG. 3E). Same results were observed in BALB/c mice injected with these vectors (FIGS. 9A-9D). AAV11 was the serotype used for all subsequent of studies.

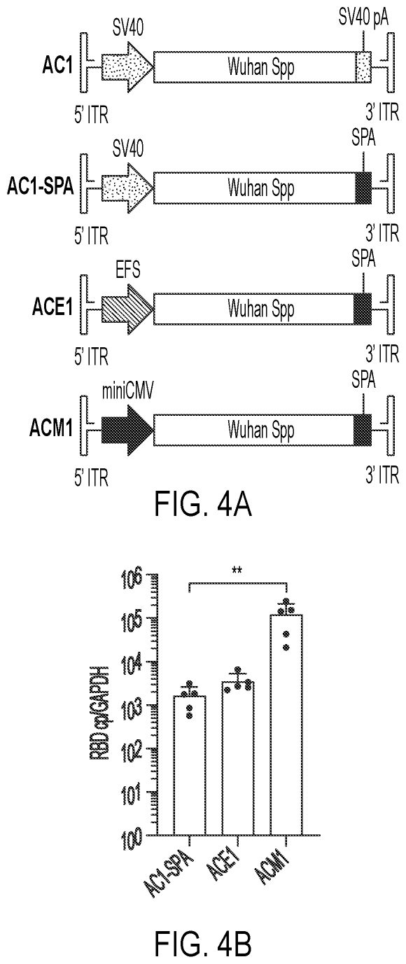

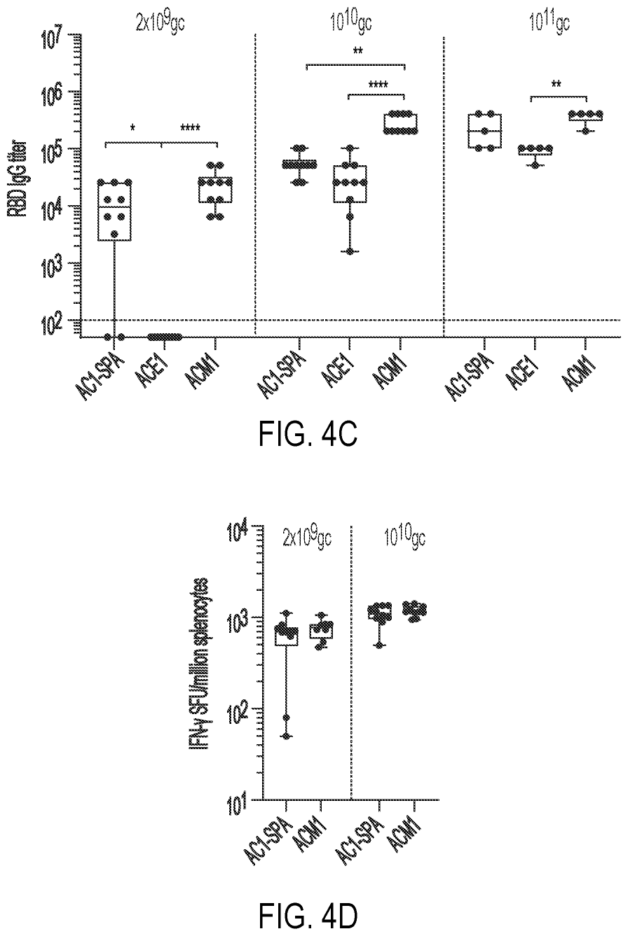

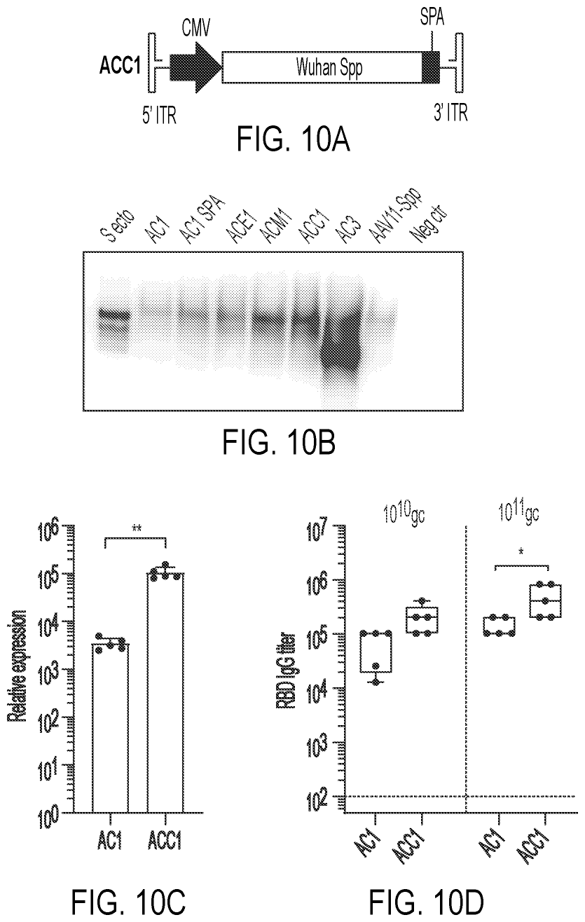

Next, the difference in seroconversion between AC1 and AC3 was noted at low doses in NHP (FIG. 2A), which was not consistent with the observations in mice or at higher doses in NHP (28). Based on the observation that the SV40 promoter in AC1 (comprising Spp antigen) was 10-100× less active compared to the CMV promoter in AC3 (comprising S1 antigen) in mouse muscle, it was possible that the level of antigen expression is a key determinant of vaccine potency (as measured by seroconversion rate and antibody levels) in addition to the quality of the immunogen (28). Thus, AAV expression cassettes were designed to improve the expression of Spp. The main limitation was the size of the recombinant AAV genome: SARS-CoV-2 Spike is 3.8 Kb, leaving <0.7 Kb space between ITRs for the regulatory elements (minimally, promoter and polyadenylation signal or polyA). The SV40 polyA in AC1 was substituted by a shorter synthetic polyA (SPA) to create AC1-SPA vector (FIG. 4A). To increase the expression of Spike, the SV40 promoter was substituted by a short EF1α promoter (EFS), a minimal CMV promoter (miniCMV) or the full CMV promoter to create ACE1, ACM1 and ACC1 vectors, respectively (FIGS. 4A and 10A). ACC1 promoter, due to the long size of the promoter, resulted in an oversized recombinant genome, which could lead to fragmented genome packaging and lower vector yields at scale (32, 33). In vitro expression studies revealed improved expression of Spike protein in cells infected with ACM1 and ACC1 compared to AC1 (FIG. 10B). This was confirmed in C57BL/6 female animals that received these candidates by measuring Spike mRNA levels in the injected muscle 7 days after a 10¹¹gc IM injection (FIGS. 4B and 10C). Higher expression resulted in significantly higher RBD-binding antibody levels in animals vaccinated with ACM1 compared to AC1-SPA and ACE1 at 3 doses ranging from 2×10⁹ gc to 10¹¹gc. Interestingly, ACM1 achieved full seroconversion with a single dose as low as 2×10⁹ gc per mouse, while 20% of AC1-SPA animals at the same dose were found non-responders by analyzing humoral and cellular immune responses (FIGS. 4C and 4D). No significant difference was found in IFN-γ ELISPOT between AC1-SPA and ACM1 (FIG. 3D). ACC1 also showed increased transduction in the injected muscle and increased antibody responses, in line with ACM1 (FIGS. 10C and 10D).

These data supported the use of the ACM vaccine design for further studies, based on the potential for higher potency and/or dose reduction.

Robust and Rapid Programmability of ACM with VOC Antigen

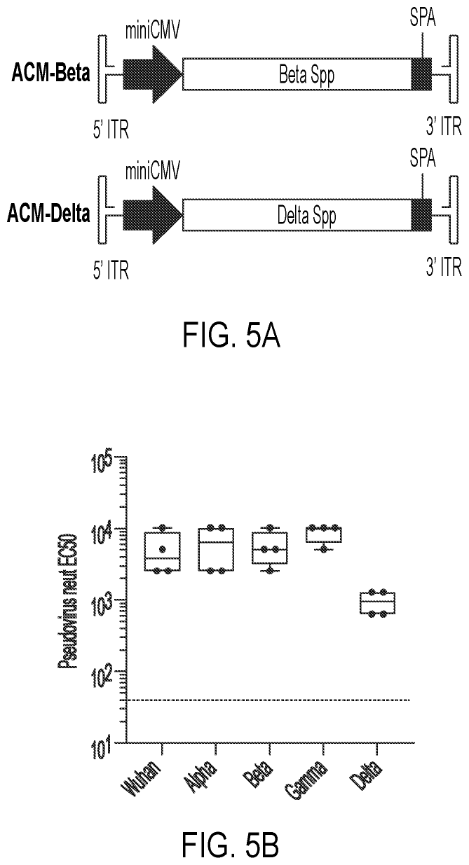

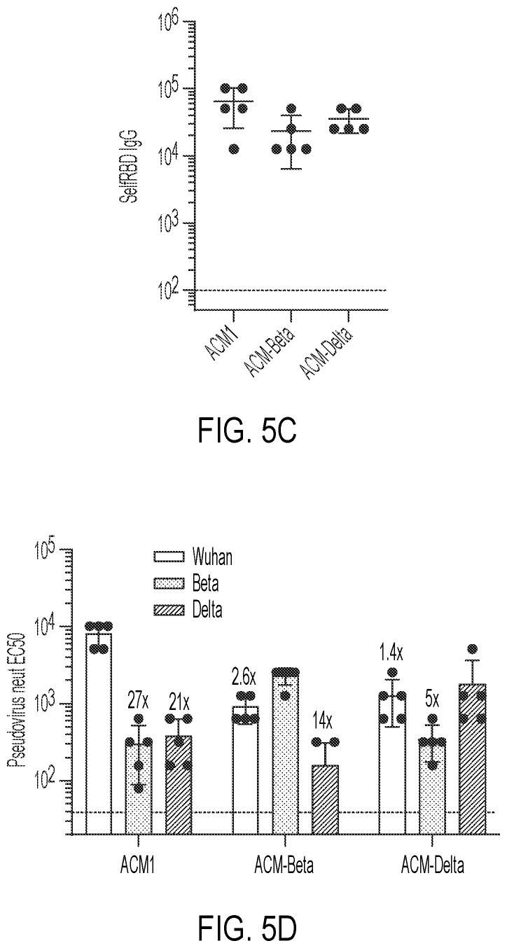

Gene-based vaccines can be designed and developed more quickly due to the programmability of their DNA template and standard production process independent of antigen. The robustness and nimbleness of the development of ACM was demonstrated with a relevant SARS-COV-2 VOC. The SARS-COV-2 Beta VOC is reported to be highly antigenically distinct to other variants, and hence is significantly less neutralized in individuals exposed to or immunized with the ancestral Wuhan Spike. Interestingly however, individuals infected with Beta may develop stronger cross-reactivity to Wuhan and most of the other VOCs (34). Therefore, the second generation AAVCOVID platform (ACM) was adapted to express the Spp of Beta (FIG. 5A). The new ACM-Beta candidate was first tested in C57BL/6 mice. All animals developed high titers of neutralizing antibodies against the self-transgene Beta and equally high neutralization of Wuhan, Alpha and Gamma VOCs (FIG. 5B). In line with prior observations, cross-neutralization was lower for the Delta VOC (35).

The ACM-Delta vaccine candidate that expresses the Delta Spp (FIG. 5A) was also recently generated. When compared side-by-side in mice, these candidates elicited the same binding antibody levels (against the self-RBD) on day 14 after vaccination (FIG. 5C). Neutralization of pseudoviruses comprising the homologous Spike on day 28 was comparable between groups, however cross-reactivity changed for each of the candidates (FIG. 5D). The Wuhan Spike on ACM1 elicited high neutralizing titers against the Wuhan Spike but the titers were >20-fold reduced against other Spike VOC. ACM-Beta neutralized Beta and Wuhan similarly but showed lower neutralization of Delta. Finally, ACM-Delta elicited the most efficient antibodies to neutralize all three Spikes.

ACM-Beta Protects from Beta SARS-COV-2 Challenge in Cynomolgus Macaques at Low Dose

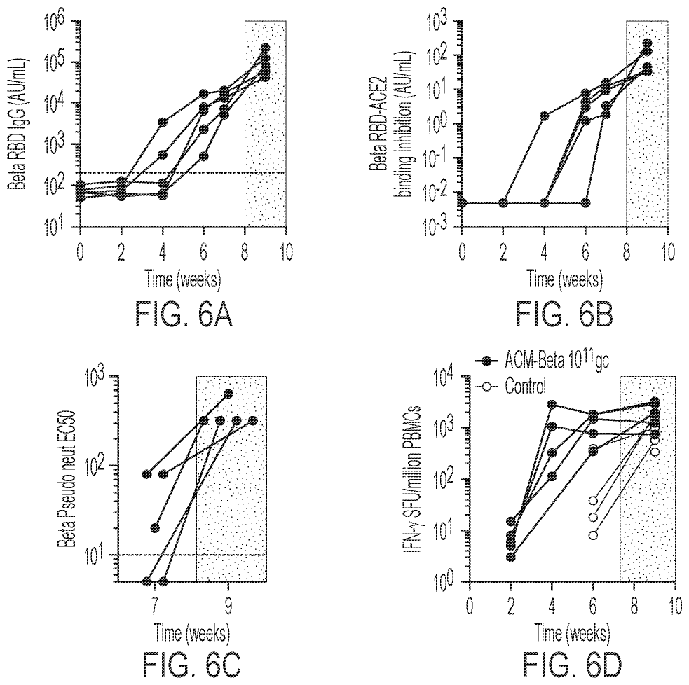

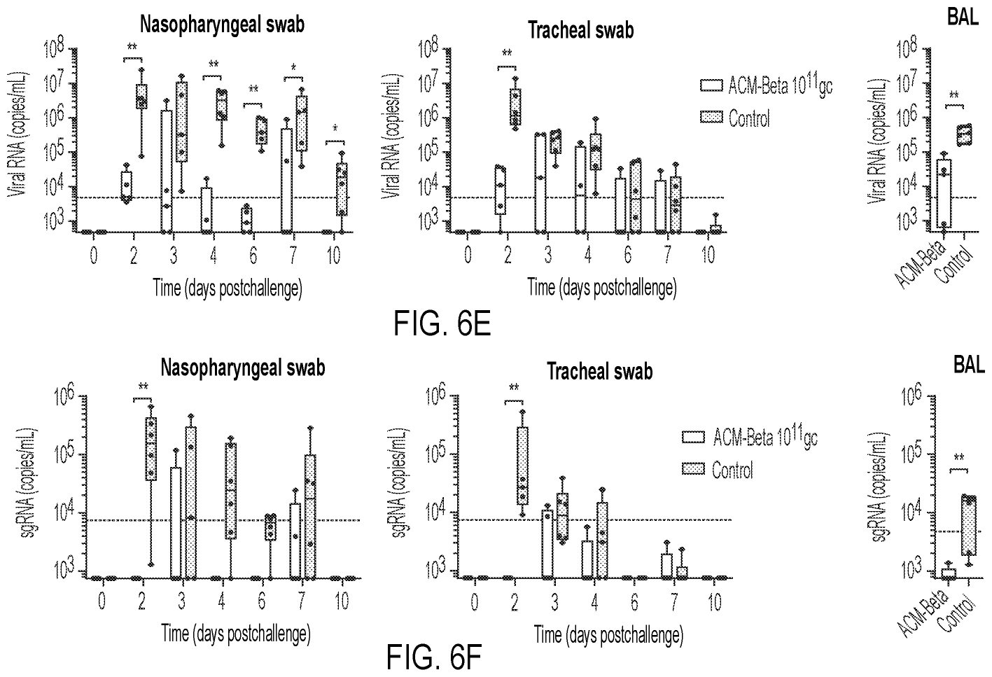

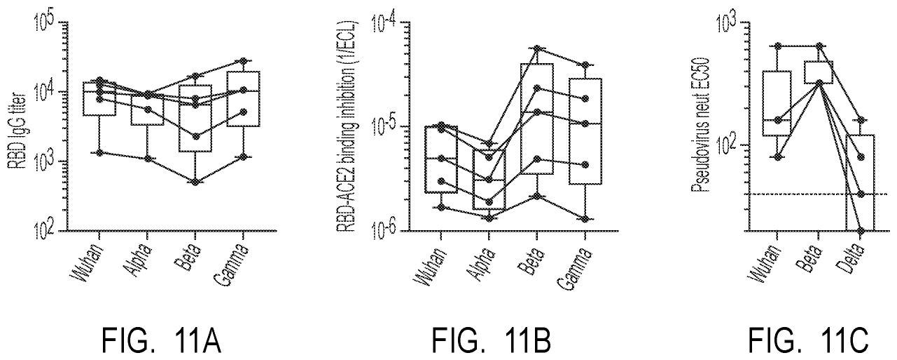

In order to evaluate the efficacy of ACM compared to AC at low dose, Cynomolgus macaques (n=5) were IM injected with 10¹¹gc ACM-Beta. Importantly, unlike AC1 in prior studies, at this dose all animals seroconverted by week 6, as measured by Beta RBD-binding antibodies (FIGS. 2A and 6A). ACE2 binding inhibition assay and pseudovirus neutralization assay showed similar trends, although like with AC1 or AC3 with a lag time in terms of kinetics (28) (FIGS. 6B and 6C). IFN-γ-mediated cellular responses were detectable and high on peripheral blood mononuclear cells (PBMCs) by week 4 (FIG. 6D). Cross-neutralization was measured by RBD-binding. ACE2 inhibition and pseudovirus assay (FIGS. 11A-11C). Binding antibody levels were very similar for different VOC RBDs (FIG. 11A), but ACE2 binding inhibition and pseudovirus neutralization were better for Beta and Gamma variants while lower for Wuhan, Alpha and Delta (FIGS. 11B and 11C).

ACM-Beta vaccinated animals and control animals were challenged with 10⁵ pfu of Beta SARS-COV-2 VOC (isolate hCoV-19/USA/MD-HP01542/2021, lineage B.1.351) administered intranasally and intratracheally. Viral and subgenomic RNA were measured in the upper and lower respiratory tracts. Viral RNA was detectable in some of the animals in some nasopharyngeal and tracheal swabs, as well as in the BAL harvested on day 3 after inoculation of the virus (FIG. 6E). However, overall viral loads were significantly lower (significantly lower area under the curve or AUC in both nasopharyngeal and tracheal viral RNA) and were cleared faster. Regarding active replication of the virus, only one animal presented sgRNA detectable above the limit of quantification on day 3 (FIG. 6F). sgRNA was not detectable in BAL samples on day 3 (FIG. 6F).

### AAVCOVID Induces Potent CD4⁺ T Cell Responses

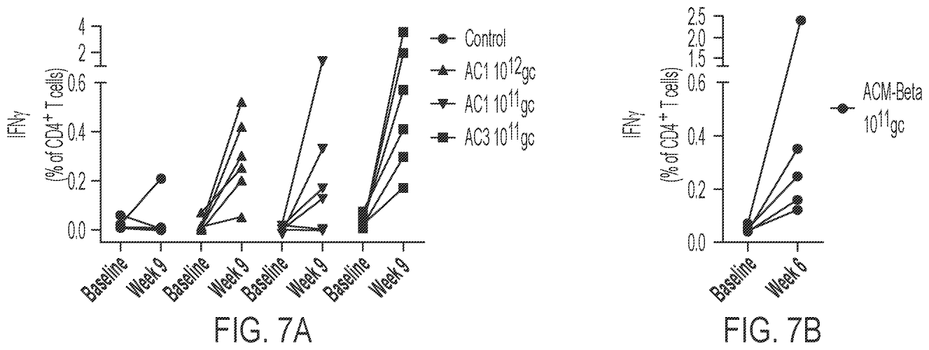

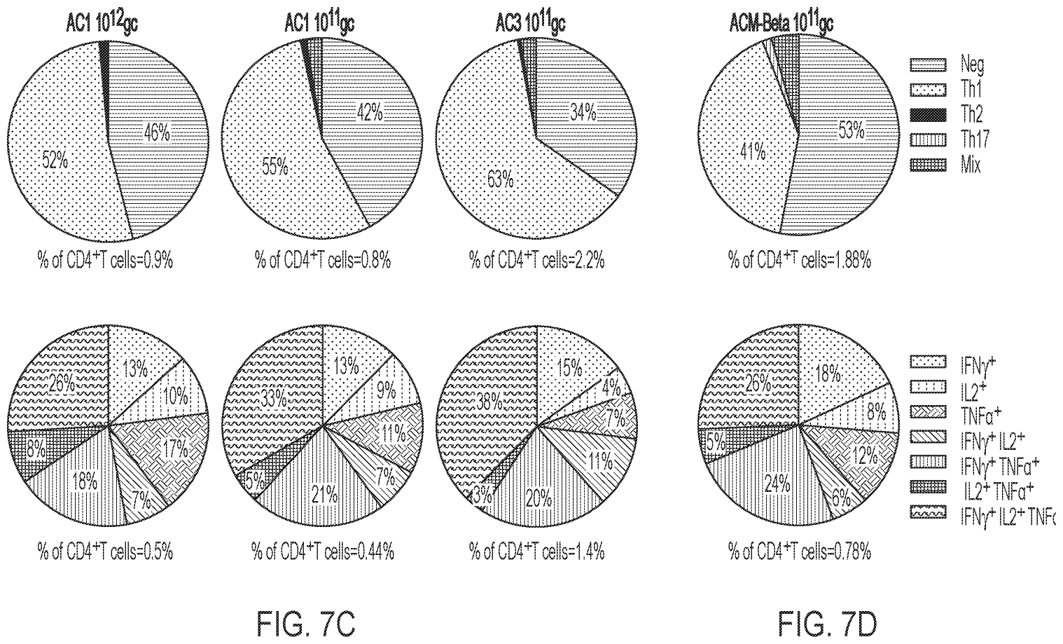

Cellular responses were measured in both NHP studies: 1) in animals vaccinated with 10¹² and 10¹¹gc of AC1 and 10¹¹gc of AC3 on week 9 after vaccination, and 2) animals vaccinated with 10¹¹gc of ACM-Beta in PBMCs extracted on week 6. All animals developed IFN-γ-secreting CD4⁺ T cells, except the 2 animals in the AC1 low dose that failed to seroconvert after vaccination (FIGS. 7A and 7B). Upon stimulation with Spike peptides percentages ranging from 0.8 to 2.2% of activated CD4+ T cells were detected by intracellular staining (ICS), and 41-63% of these activated cells presented a Th1 phenotype (secretion of IFN-γ, IL-2 and/or TNFα) (FIGS. 7C and 7D). 26-38% of these Th1 phenotype cells were polyfunctional (secretion of the 3 cytokines) and around a third secreted combinations of 2 cytokines (FIGS. 7C and 7D). These data demonstrate that AAVCOVID elicited a robust and polyfunctional cellular response.

### Discussion

The constantly evolving COVID19 pandemic requires vaccines and vaccine regimens to adapt to the rapidly changing threat. Past experience demonstrated that vaccines are indeed a key tool in managing the ongoing crisis, however rapid global deployment is needed to prevent the emergence of new variants, vaccines need to have breadth and/or adaptability to be effective against current and future VOCs, protection from disease needs to be durable, and ideally also prevent transmission. Presented herein is the evaluation and optimization of an AAV-based COVID19 vaccine platform in its potential to address some of the limitations that have been exposed.

First generation AAVCOVID candidate can fully suppress viral replication in the upper and lower respiratory tract and confer protection against SARS-COV-2 challenge in NHPs at a single 10¹² gc dose. Furthermore, immunogenicity can be maintained at peak protective antibody levels for at least 18 months. Lastly, the AAV-based vaccine product can be manufactured in a scalable process and was stable when stored for 1 month at room temperature and at least 12 weeks at 4° C.

While these desirable features of the AAVCOVID vaccine are highly relevant considering the challenges in containing the pandemic to date, a reduction of the effective dose of 10¹² gc was highly desirable, if not critical, to warrant safety, scalability and cost effectiveness. The studies here illustrate demonstrate that first-generation candidates AC1 and AC3 might not be protective in a cynomolgus macaque SARS-COV-2 challenge model. In an optimization of the AAVCOVID vaccine, largely based on correlating AC1 and AC3 relative performance with their distinct design features (mainly promotor strength), the ability to reduce doses by approximately 10-fold to a 10¹¹ gc dose was demonstrated in mouse and NHP. At these dose levels, AAVCOVID becomes a tractable vaccine platform in terms of scalability and cost.

### Materials and Methods

### NHP Studies

Rhesus (Macaca mulatta) animal study was performed by University of Pennsylvania under the approval of the Institutional Animal Care and Use Committee of the Children's Hospital of Philadelphia. Rhesus macaques (that screened negative for viral pathogens including SIV (simian immunodeficiency virus), STLV (simian-T-lymphotrophic virus), SRV (simian retrovirus), and B virus (macacine herpesvirus 1) were enrolled on the study. Animals were housed in an AAALAC International-accredited nonhuman primate research in stainless-steel squeeze back cages, on a 12-hour timed light/dark cycle, at temperatures ranging from 64-79° F. (18-26° C.). Animals received varied enrichment such as food treats, visual and auditory stimuli, manipulatives, and social interactions throughout the study. Four 3- to 7-year-old Rhesus macaques (Macaca mulatta) were treated with the clinical candidates (n=2/vector, 1 female and 1 male) intramuscularly at a dose of 10¹² gc/animal. Serum was obtained in regular intervals for several analyses of immunogenicity against SARS-COV-2 Spike.

Cynomolgus macaques (Macaca fascicularis), aged 43-45 months (14 females and 10 males) and original from Mauritian AAALAC certified breeding centers were used for SARS-CoV-2 challenge studies. All animals were housed in IDMIT facilities (CEA, Fontenay-aux-roses), under BSL-3 containment (Animal facility authorization #D92-032-02, Préfecture des Hauts de Seine, France) and in compliance with European Directive 2010/63/EU, the French regulations and the Standards for Human Care and Use of Laboratory Animals, of the Office for Laboratory Animal Welfare (OLAW, assurance number #A5826-01, US). The protocols were approved by the institutional ethical committee “Comité d′Ethique en Expérimentation Animale du Commissariat à l′Energie Atomique et aux Energies Alternatives” (CEtEA #44) under statement number A20-037. The study was authorized by the “Research, Innovation and Education Ministry” under registration number APAFIS #24434-2020030216532863 and APAFIS #28946-2021011312169043.

Cynomolgus macaques were randomly assigned to the experimental groups.

For the first study testing AC1 and AC3, the different vaccinated groups (n=6 for each) received a 10¹² gc or 10¹¹ ge of AC1 vaccine candidate or 10¹¹ gc of AC3 vaccine candidate while control animals (n=6) received only the diluent. Blood was sampled from vaccinated animals at weeks 0, 1, 2, 4, 5, 6, 7, 8 and 9. Sixty-seven days after immunization, all animals were exposed to a total dose of 10⁵ pfu of SARS-COV-2 virus (hCoV-19/France/IDF0372/2020 strain; GISAID EpiCoV platform under accession number EPI_ISL_406596) via the combination of intranasal and intra-tracheal routes (0.25 mL in each nostril and 4.5 mL in the trachea, i.e. a total of 5 mL; day 0), using atropine (0.04 mg/kg) for pre-medication and ketamine (5 mg/kg) with medetomidine (0.05 mg/kg) for anesthesia. Nasopharyngeal and tracheal swabs were collected at 2, 3, 4, 5, 8, 11, 14 and 25 days post exposure (d.p.c.) while blood was taken at 2, 3, 4, 5, 8, 11, 14, 25 and 31 d.p.c. Bronchoalveolar lavages (BAL) were performed using 50 mL sterile saline at 3 and 11 d.p.c. Pet-CT scan were performed at D5 or 6 and a CT scan was done at D14.

For the second study evaluating the ACM-Beta vaccine candidate, the vaccinated group (n=5) received a 10¹¹ gc of ACM-Beta vaccine candidate while control animals (n=6) received only diluent. Blood was sampled from vaccinated animals at weeks 0, 1. 2, 4, 5, 6 and 7. Fifty-four days after immunization, all animals were exposed to a total dose of 10⁵ pfu of Beta SARS-CoV-2 VOC (isolate hCoV-19/USA/MD-HP01542/2021, lineage B.1.351) as described above. Nasopharyngeal and tracheal swabs were collected at 2, 3, 4, 6, 7, 10 and 14 days post exposure (d.p.c.) while blood was taken at 2, 3, 4, 7, 10 and 14 days, Bronchoalveolar lavages (BAL) were performed using 50 mL sterile saline at 3 and 11 d.p.e. CT scans were performed at D3 and D7 to quantify lung lesions.

Blood cell counts, hemoglobin and hematocrit were determined from EDTA blood using a DXH800 analyzer (Beckman Coulter).

### Mouse Studies

Mouse studies and protocols were approved by the Schepens Eye Research Institute IACUC. C57BL/6 and BALB/c mice were injected intramuscularly (IM) in the right gastrocnemius with different doses of vaccine candidates. Blood was harvested by submandibular bleeds and serum isolated. Several tissues were harvested at necropsy for splenocyte extraction and for biodistribution and transgene expression analyses.

### Vaccine Candidates

First generation AAVCOVID candidates were described and characterized previously (28). Second generation candidates (ACM1, ACM-Beta and ACM-Delta) consist of the AAV11 vector that expresses the codon optimized, pre-fusion stabilized (furin cleavage site mutated to G₆₈₂SAS₆₈₅ and P₉₈₆P₉₈₇ substitutions) full length SARS-COV-2 Spike protein (Wuhan, Beta and Delta Spike) under the control of a minimal CMV promoter and a small synthetic polyA. Vectors were produced as previously described (28).

### In Vitro Infection and Spike Expression by Western Blot

5×10⁴ HuH7 cell/well were seeded in 12-well plates and incubated overnight at 37° C. On the following day, cells were pre-incubated for 2 hours with adenovirus 5 (Ad5) at a MOI of 20 pfu/cell, and infected with a MOI of 5×10⁵ of AC1 or AC3. Cells were harvested 72 hours later and lysed with NuPAGE™ LDS Sample Buffer (4×) (Thermo Fisher Scientific, Cat #NP0007) at 99° C. for 5 minutes. Proteins were separated by electrophoresis in NuPAGE 4-12% polyacrylamide gels (Thermo Fisher Scientific, Cat #NP0321PK2) and then transferred to PVDF membranes. The membranes were probed with an anti-SARS-COV-2 RBD rabbit polyclonal antibody (Sino Biological Inc., 40592-T62) followed by a goat anti-rabbit HRP-conjugated secondary antibody (Thermo Fisher Scientific, Cat #A16110, RRID AB_2534782). Membranes were developed by chemiluminescence using the Immobilon Western Chemiluminescent HRP Substrate (Millipore, Cat #WBKLS0500) and recorded using ChemiDoc MP Imaging System (Bio-Rad). An anti-GAPDH antibody (Cell Signaling Technology Cat #2118, RRID: AB_561053) was used as loading control.

### Quantification of Antibodies by Mesoscale

Cynomolgus macaque samples were screened for spike and RBD-specific IgG and their neutralizing capacity (analyzed by a pseudo-neutralizing spike-ACE2 assay) against SARS-CoV-2 wild-type and variants B.1.1.7, B.1.351 and P.1 using the V-PLEX SARS-COV-2 Panel 7 (IgG and ACE2, MesoScale Discovery (MSD), Rockville, USA) according to the manufacturer's instructions and as previously described (36). The plates were blocked with 50 μl of blocker A (1% BSA in MilliQ water) solution for at least 30 min at room temperature shaking at 700 rpm with a digital microplate shaker. During blocking, heat-inactivated serum samples were diluted 1:500 and 1:5000 (IgG assay) or 1:10 and 1:100 (ACE2 assay) in diluent buffer. Each plate contained duplicates of a 7-point calibration curve with serial dilution of a reference standard, and a blank well. The plates were then washed three times with 150 μl of the MSD kit Wash Buffer, blotted dry, and 50 μl (IgG assay) or 25 μl (ACE2 assay) of the diluted samples were added to the plates and set to shake at 700 rpm at room temperature for at least 2 h. The plates were again washed three times and 50 μl of SULFO-Tagged anti-Human IgG antibody or 25 μl SULFO-Tagged human ACE2 protein, respectively, was added to each well and incubated shaking at 700 rpm at room temperature for at least 1 h. Plates were then washed three times and 150 μl of MSD GOLD Read Buffer B was added to each well. The plates were read immediately after on a MESO QuickPlex SQ 120 machine. Electro-chemioluminescence (ECL) signal was recorded and results expressed as AU/mL.

### RBD-Binding Antibody ELISA

Nunc MaxiSorp™ high protein-binding capacity 96 well plates (Thermo Fisher Scientific, Cat #44-2404-21) were coated overnight at 4° C. with 1 μg/ml SARS-COV-2 RBD diluted in phosphate-buffered saline (PBS). The next day the plates were washed with PBS-Tween 20 0.05% (Sigma, Cat #P2287-100ML) using the Biotek 405 TS Microplate washer. Each plate was washed five times with 200 μl wash buffer and then dried before the next step. Following the first wash, 200 μl of Blocker Casein in PBS (Thermo Fisher Scientific, Cat #37528) were added to each well and incubated for 2 hours at RT. After blocking, serum samples were serially diluted in blocking solution starting into 1:100 dilution. Rhesus BAL samples were added undiluted and serially diluted in blocking solution. After an hour of incubation, the plates were washed and 100 μl of secondary Peroxidase AffiniPure Rabbit Anti-Mouse IgG (Jackson ImmunoResearch, Cat #315-035-045, RRID: AB_2340066) antibody diluted 1:1,000 in blocking solution was added to each well. After one hour of incubation at room temperature, the plates were washed and developed for 3.5 min with 100 μl of Seracare SureBlue Reserve™ TMB Microwell Peroxidase Substrate solution (SeraCare, Cat #53-00-03). The reaction was then stopped with 100 μl Seracare KPL TMB Stop Solution (SeraCare, Cat #50-85-06). Optical density (OD) at 450 nm was measured using a Biotek Synergy H1 plate reader. The titer was the reciprocal of the highest dilution with absorbance values higher than four times the average of the negative control wells.

### Pseudovirus Neutralizing Assay

This assay was performed as previously described (28). Briefly, pseudo-lentiviruses were produced by triple transfection of psPAX2, pCMV-SARS2-Spike (WT or VOC) and pCMV-Lenti-Luc in HEK293T cells. After 48 hours, the supernatant of the cells was harvested, centrifuged at 4,000 rpm at 4° C. for 5 minutes and filtered through 0.45 μm filter. Pseudovirus TCID50 was calculated by limiting dilution in HEK293T-ACE2 cells. For the neutralization assay, serial dilutions of sera were incubated with the pseudovirus for 45 minutes at 37° C., and subsequently added to HEK293T-ACE2 cells. 48 hours later luciferase signal was measured to calculate the EC50 values for each serum sample.

### IFN-γ and IL-4 ELISPOT Assay in Mouse

IFN-γ and IL-4 ELISPOT were performed in mouse splenocytes as previously described (37). Briefly, 10 μg/ml anti-mouse IFN-γ ELISPOT capture antibody (BD Biosciences Cat #551881, RRID: AB_2868948) or 4 μg/ml anti-mouse IL-4 ELISPOT capture antibody (BD Biosciences Cat #551878, RRID: AB_2336921) were used as capture antibody. One million of freshly isolated splenocytes were seeded into the precoated plates and stimulated with S1 and S2 peptides pools (GenScript) with a final concentration of 1 μg/ml of each peptide diluted in RPMI-1640 supplemented with 10% FBS and incubated for 48 hours at 37° C. with 5% CO₂. Each peptide pool, consisting of 15-mers peptides overlapping by 10 amino acids, spanning the entire SARS-COV-2 Spike protein S1 or S2 subunits. Control wells contained 5×10⁵ cell stimulated with DMSO diluted in RPMI-1640 supplemented with 10% FBS (negative control) or 2 μg/ml concanavalin A (positive control). Subsequently, the plates were washed and incubated with biotin-conjugated mouse IFN-γ ELISPOT Detection Antibody (BD Biosciences Cat #551881, RRID: AB_2868948) and 4 μg/ml biotin-conjugated mouse IL-4 detection antibody (BD Biosciences Cat #551878, RRID: AB_2336921) at room temperature for 3 hours and followed by streptavidin-HRP (dilution 1:1000, Sigma-Aldrich, Cat #18-152) for 45 minutes. After washing, 100 μL/well of NBT/BCIP substrate solution (Promega, Cat #S3771) were added and developed for 15-30 min until distinct spots emerged. The cytokine-secreting cell spots were imaged and counted on AID EliSpot reader (Autoimmun Diagnostika GmbH).

### IFN-γ ELISPOT Assay in NHP PBMCs

IFNγ ELISpot assay was performed in cynomolgus macaque PBMCs using the Monkey IFNg ELISpot PRO kit (Mabtech, #3421M-2APT) according to the manufacturer's instructions. PBMCs were plated at a concentration of 200,000 cells per well and were stimulated with Wuhan or Beta SARS-COV-2 spike peptides (PepMix™) synthetized by JPT Peptide Technologies (Berlin, Germany). These 15-mer peptides are divided in two pools (S1 and S2) of respectively 158 and 157 peptides overlapping by 11 amino acids. The peptides are coding for the S protein of SARS-COV-2 and will be used at a final concentration of 2 μg/mL. Plates were incubated for 18 h at 37 C in an atmosphere containing 5% CO2, then washed 5 times with PBS and incubated for 2 h at 37 C with a biotinylated anti-IFNγ antibody. After 5 washes, spots were developed by adding 0.45 mm-filtered ready-to-use BCIP/NBT-plus substrate solution and counted with an automated ELISpot reader ELRIFL04 (Autoimmun Diagnostika GmbH, Strassberg, Germany). Spot forming units (SFU) per 106 PBMCs are means of duplicates wells for each stimulation and each animal.

### Intracellular Staining in PBMCs

T-cell responses were characterized by measurement of the frequency of PBMC expressing IL-2 (PerCP5.5, 1:10; #560708; MQ1-17H12, BD), IL-17a (Alexa700, 1:20; #560613; N49-653, BD), IFN-γ (V450, 1:33.3; #560371; B27, BD), TNF-α (BV605, 1:30.3; #502936; Mab11, BioLegend), IL-13 (BV711, 1:20; #564288; JES10-5A2, BD), CD137 (APC, 1:20; #550890; 4B4, BD) and CD154 (FITC, 1:20; #555699; TRAP1, BD) upon stimulation with the two Wuhan SARS-COV-2 PepMix™) synthetized by JPT Peptide Technologies (Berlin, Germany). peptide pools. CD3 (APC-Cy7, 1:200; #557757; SP34-2, BD), CD4 (BV510, 1:33.3; #563094; L200, BD) and CD8 (PE-Vio770, 1:50; #130-113-159; BW135/80, Miltenyi Biotec) antibodies was used as lineage markers. One million of PBMC were cultured in complete medium (RPMI1640 Glutamax+, Gibco; supplemented with 10% FBS), supplemented with co-stimulatory antibodies (FastImmune CD28/CD49d, Becton Dickinson). Then cells were stimulated with S sequence overlapping peptide pools at a final concentration of 2 μg/mL. Brefeldin A was added to each well at a final concentration of 10 μg/mL and the plate was incubated at 37° C., 5% CO2 during 18 h. Next, cells were washed, stained with a viability dye (LIVE/DEAD fixable Blue dead cell stain kit, ThermoFisher), and then fixed and permeabilized with the BD Cytofix/Cytoperm reagent. Permeabilized cell samples will be stored at −80° C. before the staining procedure. Antibody staining was performed in a single step following thawing. After 30 min of incubation at 4° C., in the dark, cells were washed in BD Perm/Wash buffer then acquired on the LSRII flow cytometer (BD). Analysis was performed with FlowJo v.10 software.

### SARS-COV-2 Genomic and Subgenomic RNA RT-qPCR

Upper respiratory (nasopharyngeal and tracheal) specimens were collected with swabs (Viral Transport Medium, CDC, DSR-052-01). Tracheal swabs were performed by insertion of the swab above the tip of the epiglottis into the upper trachea at approximately 1.5 cm of the epiglottis. All specimens were stored between 2° C. and 8° C. until analysis by RT-qPCR with a plasmid standard concentration range containing an RdRp gene fragment including the RdRp-IP4 RT-PCR target sequence. The limit of detection was estimated to be 2.67 log₁₀ copies of SARS-COV-2 gRNA per mL and the limit of quantification was estimated to be 3.67 log₁₀ copies per mL. SARS-COV-2 E gene subgenomic mRNA (sgRNA) levels were assessed by RT-qPCR using primers and probes previously described (38, 39): leader-specific primer sgLeadSARSCOV2-F CGATCTCTTGTAGATCTGTTCTC (SEQ ID NO: 7), E-Sarbeco-R primer ATATTGCAGCAGTACGCACACA (SEQ ID NO: 8) and E-Sarbeco probe HEX-ACACTAGCCATCCTTACTGCGCTTCG-BHQ1 (SEQ ID NO: 9). The protocol describing the procedure for the detection of SARS-COV-2 is available on the WHO website (who.int/docs/default-source/coronaviruse/real-time-rt-pcr-assays-for-the-detection-of-sars-cov-2-institut-pasteur-paris.pdf?sfvrsn=3662fcb6_2). The limit of detection was estimated to be 2.87 log₁₀ copies of SARS-COV-2 sgRNA per mL and the limit of quantification was estimated to be 3.87 log₁₀ copies per mL.

### [¹⁸F]-FDG PET/CT Protocol

All imaging acquisitions were performed on the Digital Photon Counting (DPC) PET-CT system (Vereos-Ingenuity, Philips) (40) implemented in BSL3 laboratory.

For imaging sessions, animals were first anesthetized with Ketamine (10 mg/kg)+Metedomidine (0.05 mg/kg) and then maintained under isofluorane 2% in a supine position on a patient warming blanket (Bear Hugger, 3M) on the machine bed with cardiac rate, oxygen saturation and temperature monitoring.

CT was performed under breath-hold 5 minutes prior to PET scan for attenuation correction and anatomical localization. The CT detector collimation used was 64×0.6 mm, the tube voltage was 120 kV and intensity of about 150 mAs. Automatic dose optimization tools (Dose Right, Z-DOM, 3D-DOM by Philips Healthcare) regulated the intensity. CT images were reconstructed with a slice thickness of 1.25 mm and an interval of 0.25 mm.

A whole-body PET scan (4-5 bed positions, 3 min/bed position) was performed 45 min post injection of 3.39±0.28 MBq/kg of [¹⁸F]-FDG via the saphenous vein. PET images were reconstructed onto a 256×256 matrix (3 iterations, 17 subsets).

Images were analyzed using INTELLISPACE PORTAL 8 (Philips healthcare) and 3DSlicer (Open source tool). Different regions of interest (lung and lung draining lymph nodes) were defined by CT and PET. Pulmonary lesions were defined as Ground Glass Opacity, Crazy-paving pattern or consolidation as previously described (41-43). Lesion features detected by CT imaging were assessed by two analyzers independently and final CT score results were obtained by consensus.

Besides, regions with FDG uptake (lung, lung draining lymph nodes and spleen) were also defined for quantification of SUV parameters, including SUVmean, SUVmax.

### Lung Histopathological Analysis and Scoring

At necropsy, cranial and caudal lobes of the lungs were fixed by immersion in 10% formaline solution for 24 hours. Samples were formaline fixed paraffin embedded (FFPE) with vacuum inclusion processor (Excelsior, Thermo) and cut in 5 μm (Microtome RM2255, Leica) slices mounted on coated glass slides (Superfrost+, Thermo) and stained with haematoxylin and eosin (H&E) with automated staining processor (Autostainer ST5020, Leica).

Each slide was scored in 20 different spots at ×40 magnification (Plan Apo □40X, 0.95 Numerical aperture, 0.86 mm2 per Field of View). On each spot, 5 different parameters were assessed: Septal cellularity, Septal fibrosis, Type II pneumocytes hyperplasia and alveolar neutrophils. A systematic histopathology scoring was used and described in Table below. Each score was then cumulated for each assessed field of view for cranial and caudal lobes.

| Parameters | Score | Description |
|---|---|---|
| Septal cellularity | 0 | Normal alveolar wall (AW) |
| | 1 | 1-2 red blood cell (RBC), thick AW |
| | 2 | 2-4 RBC, thick AW |
| | 3 | 5-10 RBC, thick AW |
| | 4 | >10, thick AW |
| Septal fibrosis | 0 | Absent |
| | 1 | Present |
| Type II pneumocytes | 0 | Absent |
| hyperplasia | 1 | Present |
| Alveolar macrophages | 0 | Absent |
| | 1 | 1-5 per x20 field of view (FOV) |
| | 2 | 6-10 per x20 FOV |
| | 3 | 11-15 per x20 FOV |
| | 4 | >15 per x20 FOV |
| Alveolar neutrophils | 0 | Absent |
| | 1 | 1-5 per x20 FOV |
| | 2 | 6-10 per x20 FOV |
| | 3 | 11-15 per x20 FOV |
| | 4 | >15 per x20 FOV |

### Biodistribution/Gene Expression Studies

Tissue collection was segregated for genomic DNA (gDNA) or total RNA work by QIASymphony nucleic acid extraction with the aim of filling up 96-well plates of purified material. A small cut of frozen tissue (˜ 20 mg) was used for all extractions with the exception of gDNA purifications from spleen (1-2 mg). Tissues were disrupted and homogenized in QIAGEN Buffer ATL (180 μL) and lysed overnight at 56° C. in the presence of QIAGEN Proteinase K (400 μg) for gDNA, or directly in QIAGEN® Buffer RLT-Plus in the presence of 2-mercaptoethanol and a QIAGEN anti-foaming agent for total RNA purification. Tissue lysates for gDNA extraction were treated in advance with QIAGEN RNase A (400 μg), while tissue homogenates for RNA extraction were DNase-I treated in situ in the QIASymphony® during the procedure. Nucleic acids were quantified only if necessary, as a troubleshooting measure. Purified gDNA samples were diluted 10-fold and in parallel into Cutsmart-buffered BamHI-HF (New England Biolabs) restriction digestions in the presence of 0.1% Pluronic F-68 (50 μL final volume) that ran overnight prior to quantification. Similarly, DNase-I-treated total RNAs were diluted 10-fold into cDNA synthesis reactions (20 μL final volume) with or without reverse transcriptase using the High Capacity cDNA Reverse Transcription Kit (Thermo Fisher™). For ddPCR (gDNA or cDNA) or qPCR (cDNA), 2 μL of processed nucleic acids were used for quantification using Bio-Rad™ or Applied Biosystems™ reagents, respectively, in 20 μL reactions using default amplification parameters without an UNG incubation step. All the studies included negative control (PBS) groups for comparison. The significantly small variance of multiple technical replicates in ddPCR justified the use of a single technical replicate per sample and no less than three biological replicates per group, gender, or time point. coRBD signal for ddPCR and vector biodistribution (gDNA) was multiplexed and normalized against the mouse transferrin receptor (Tfrc) gene TaqMan™ assay using a commercial preparation validated for copy number variation analysis (Thermo Fisher Scientific). Likewise, coRBD signal for ddPCR and gene expression analysis was multiplexed and normalized against the mouse GAPDH gene, also using a commercial preparation of the reference assay (Thermo Fisher Scientific). Target and reference oligonucleotide probes are tagged with different fluorophores at the 5′-end which allows efficient signal stratification. For qPCR, coRBD and mGAPDH TaqMan assays were run separately to minimize competitive PCR multiplexing issues prior to analysis and delta delta Ct normalization (44). The limit of detection of the assay was 10 copies/reaction, therefore, wells with less than 10 copies were considered negative.

### Statistical Analysis

GraphPad Prism 9 was used for graph preparation and statistical analysis. Groups were compared using Kruskal Wallis and Dunn's test. Pairs of groups were compared using Student's t test (independent samples, n>10) and Mann Whitney's U (independent samples, n<10).

Additional Sequences
pACC1

| | | |
|---|---|---|
| 5′-ITR | 1..141 | 141 |
| Full CMV promotor | 222..804 | 583 |
| Wuhan Spike Stabilized | 825..4646 | 3822 |
| SP | 825..863 | 39 |
| S1 | 864..2876 | 2013 |
| RBD | 1782..2447 | 666 |
| Furin Recognition Site | 2869..2876 | 8 |
| Proline substitution | 3785..3785 | 1 |
| poly(A) signal | 4656..4704 | 49 |
| 3′-ITR | 4792..4921 | 130 |
| KanR | 5685..6494 | 810 |
| Ori | 6665..7253 | 589 |

cCTTAATTAGGCtgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatgattaacccgccatgctacttat
ctacgtagccatgctctaggaagatcggaattcgcccttaagctagctagttattaatagtaatcaattacggggtcattagttcatagcccata
tatggagttccgcgttacataacttacggtaaatggcccgcctggctgaccgcccaacgacccccgcccattgacgtcaataatgacgtatg
ttcccatagtaacgccaatagggactttccattgacgtcaatgggtggagtatttacggtaaactgcccacttggcagtacatcaagtgtatca
tatgccaagtacgccccctattgacgtcaatgacggtaaatggcccgcctggcattatgcccagtacatgaccttatgggactttcctacttgg
cagtacatctacgtattagtcatcgctattaccatggtgatgcggttttggcagtacatcaatgggcgtggatagcggtttgactcacggggat
ttccaagtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagagctggtttagtgaaccgtgcggccgcctagatgccaccaT
GTTCGTCTTCCTGGTCCTGCTGCCTCTGGTCTCCTCACAGTGCGTCAATCTGACAACT
CGGACTCAGCTGCCACCTGCTTATACTAATAGCTTCACCAGAGGCGTGTACTATCCT
GACAAGGTGTTTAGAAGCTCCGTGCTGCACTCTACACAGGATCTGTTTCTGCCATTC
TTTAGCAACGTGACCTGGTTCCACGCCATCCACGTGAGCGGCACCAATGGCACAAA
GCGGTTCGACAATCCCGTGCTGCCTTTTAACGATGGCGTGTACTTCGCCTCTACCGA
GAAGAGCAACATCATCAGAGGCTGGATCTTTGGCACCACACTGGACTCCAAGACAC
AGTCTCTGCTGATCGTGAACAATGCCACCAACGTGGTCATCAAGGTGTGCGAGTTCC
AGTTTTGTAATGATCCCTTCCTGGGCGTGTACTATCACAAGAACAATAAGAGCTGGA
TGGAGTCCGAGTTTAGAGTGTATTCTAGCGCCAACAACTGCACATTTGAGTACGTGA
GCCAGCCTTTCCTGATGGACCTGGAGGGCAAGCAGGGCAATTTCAAGAACCTGAGG
GAGTTCGTGTTTAAGAATATCGACGGCTACTTCAAAATCTACTCTAAGCACACCCCC
ATCAACCTGGTGCGCGACCTGCCTCAGGGCTTCAGCGCCCTGGAGCCCCTGGTGGA
TCTGCCTATCGGCATCAACATCACCCGGTTTCAGACACTGCTGGCCCTGCACAGAAG
CTACCTGACACCCGGCGACTCCTCTAGCGGATGGACCGCCGGCGCTGCCGCCTACT
ATGTGGGCTACCTCCAGCCCCGGACCTTCCTGCTGAAGTACAACGAGAATGGCACC
ATCACAGACGCAGTGGATTGCGCCCTGGACCCCCTGAGCGAGACAAAGTGTACACT
GAAGTCCTTTACCGTGGAGAAGGGCATCTATCAGACATCCAATTTCAGGGTGCAGC
CAACCGAGTCTATCGTGCGCTTTCCTAATATCACAAACCTGTGCCCATTTGGCGAGG
TGTTCAACGCAACCCGCTTCGCCAGCGTGTACGCCTGGAATAGGAAGCGGATCAGC
AACTGCGTGGCCGACTATAGCGTGCTGTACAACTCCGCCTCTTTCAGCACCTTTAAG
TGCTATGGCGTGTCCCCCACAAAGCTGAATGACCTGTGCTTTACCAACGTCTACGCC
GATTCTTTCGTGATCAGGGGCGACGAGGTGCGCCAGATCGCCCCCGGCCAGACAGG
CAAGATCGCAGACTACAATTATAAGCTGCCAGACGATTTCACCGGCTGCGTGATCG
CCTGGAACAGCAACAATCTGGATTCCAAAGTGGGCGGCAACTACAATTATCTGTAC
CGGCTGTTTAGAAAGAGCAATCTGAAGCCCTTCGAGAGGGACATCTCTACAGAAAT
CTACCAGGCCGGCAGCACCCCTTGCAATGGCGTGGAGGGCTTTAACTGTTATTTCCC
ACTCCAGTCCTACGGCTTCCAGCCCACAAACGGCGTGGGCTATCAGCCTTACCGCGT
GGTGGTGCTGAGCTTTGAGCTGCTGCACGCCCCAGCAACAGTGTGCGGCCCCAAGA
AGTCCACCAATCTGGTGAAGAACAAGTGCGTGAACTTCAACTTCAACGGCCTGACC
GGCACAGGCGTGCTGACCGAGTCCAACAAGAAGTTCCTGCCATTTCAGCAGTTCGG
CAGGGACATCGCAGATACCACAGACGCCGTGCGCGACCCACAGACCCTGGAGATCC
TGGACATCACACCCTGCTCTTTCGGCGGCGTGAGCGTGATCACACCCGGCACCAAT
ACAAGCAACCAGGTGGCCGTGCTGTATCAGGACGTGAATTGTACCGAGGTGCCCGT
GGCTATCCACGCCGATCAGCTGACCCCAACATGGCGGGTGTACAGCACCGGCTCCA
ACGTCTTCCAGACAAGAGCCGGATGCCTGATCGGAGCAGAGCACGTGAACAATTCC
TATGAGTGCGACATCCCAATCGGCGCCGGCATCTGTGCCTCTTACCAGACCcagacaaac
tctcccggaagcgccagtagcgtggcctcccagTCTATCATCGCCTATACCATGTCCCTGGGCGCCGAG
AACAGCGTGGCCTACTCTAACAATAGCATCGCCATCCCAACCAACTTCACAATCTCT
GTGACCACAGAGATCCTGCCCGTGTCCATGACCAAGACATCTGTGGACTGCACAAT
GTATATCTGTGGCGATTCTACCGAGTGCAGCAACCTGCTGCTCCAGTACGGCAGCTT
TTGTACCCAGCTGAATAGAGCCCTGACAGGCATCGCCGTGGAGCAGGATAAGAACA
CACAGGAGGTGTTCGCCCAGGTGAAGCAAATCTACAAGACCCCCCCTATCAAGGAC
TTTGGCGGCTTCAATTTTTCCCAGATCCTGCCTGATCCATCCAAGCCTTCTAAGCGG
AGCTTTATCGAGGACCTGCTGTTCAACAAGGTGACCCTGGCCGATGCCGGCTTCATC
AAGCAGTATGGCGATTGCCTGGGCGACATCGCAGCCAGGGACCTGATCTGCGCCCA
GAAGTTTAATGGCCTGACCGTGCTGCCACCCCTGCTGACAGATGAGATGATCGCAC
AGTACACAAGCGCCCTGCTGGCCGGCACCATCACATCCGGATGGACCTTCGGCGCA
GGAGCCGCCCTCCAGATCCCCTTTGCCATGCAGATGGCCTATAGGTTCAACGGCATC
GGCGTGACCCAGAATGTGCTGTACGAGAACCAGAAGCTGATCGCCAATCAGTTTAA
CTCCGCCATCGGCAAGATCCAGGACAGCCTGTCCTCTACAGCCAGCGCCCTGGGCA
AGCTCCAGGATGTGGTGAATCAGAACGCCCAGGCCCTGAATACCCTGGTGAAGCAG
CTGAGCAGCAACTTCGGCGCCATCTCTAGCGTGCTGAATGAcatcctgagccggctggacccacc
ggaggcagaggtgcagatcGACCGGCTGATCACCGGCCGGCTCCAGAGCCTCCAGACCTATGT
GACACAGCAGCTGATCAGGGCCGCCGAGATCAGGGCCAGCGCCAATCTGGCAGCA
ACCAAGATGTCCGAGTGCGTGCTGGGCCAGTCTAAGAGAGTGGACTTTTGTGGCAA
GGGCTATCACCTGATGTCCTTCCCTCAGTCTGCCCCACACGGCGTGGTGTTTCTGCA
CGTGACCTACGTGCCCGCCCAGGAGAAGAACTTCACCACAGCCCCTGCCATCTGCC
ACGATGGCAAGGCCCACTTTCCAAGGGAGGGCGTGTTCGTGTCCAACGGCACCCAC
TGGTTTGTGACACAGCGCAATTTCTACGAGCCCCAGATCATCACCACAGACAACAC
CTTCGTGAGCGGCAACTGTGACGTGGTCATCGGCATCGTGAACAATACCGTGTATG
ATCCACTCCAGCCCGAGCTGGACAGCTTTAAGGAGGAGCTGGATAAGTATTTCAAG
AATCACACCTCCCCTGACGTGGATCTGGGCGACATCAGCGGCATCAATGCCTCCGT
GGTGAACATCCAGAAGGAGATCGACCGCCTGAACGAGGTGGCTAAGAATCTGAAC
GAGAGCCTGATCGACCTCCAGGAGCTGGGCAAGTATGAGCAGTACATCAAGTGGCC
CTGGTACATCTGGCTGGGCTTCATCGCCGGCCTGATCGCCATCGTGATGGTGACCAT
CATGCTGTGCTGTATGACATCCTGCTGTTCTTGCCTGAAGGGCTGCTGTAGCTGTGG
CTCCTGCTGTAAGTTTGACGAGGATGACTCTGAACCTGTGCTGAAGGGCGTGAAGC
TGCACTACACCTAAATCGgatctAATAAAATATCTTTATTTTCATTACATCTGTGTGTTG
GTTTTTTGTGTGctcgagttaagggcgaattcccgataaggatcttcctagagcatggctacgtagataagtagcatggcgggtt
aatcattaactacaaggaacccctagtgatggagttggccactccctctctgcgcgctcgctcgctcactgaggccgggcgaccaaaggtc
gcccgacgcccgggctttgcccgggcggcctcagtgagcgagcgagcgcgcagccttaattaacctaattcactggccgtcgttttacaa
cgtcgtgactgggaaaaccctggcgttacccaacttaatcgccttgcagcacatccccctttcgccagctggcgtaatagcgaagaggccc
gcaccgatcgcccttcccaacagttgcgcagcctgaatggcgaatgggacgcgccctgtagcggcgcattaagcgcggcgggtgtggtg
gttacgcgcagcgtgaccgctacacttgccagcgccctagcgcccgctcctttcgctttcttcccttcctttctcgccacgttcgccggctttcc
ccgtcaagctctaaatcgggggctccctttagggttccgatttagtgctttacggcacctcgaccccaaaaaacttgattagggtgatggttca
cgtagtgggccatcgccctgatagacggtttttcgccctttgacgttggagtccacgttctttaatagtggactcttgttccaaactggaacaac
actcaaccctatctcggtctattcttttgatttataagggattttgccgatttcggcctattggttaaaaaatgagctgatttaacaaaaatttaa
cgcgaattttaacaaaatattaacgtttataatttcaggtggcatctttcggggaaatgtgcgcggaacccctatttgtttatttttctaaataca
ttcaaatatgtatccgctcatgagacaataaccctgataaatgcttcaataatattgaaaaaggaagagtATGAGCCATATTCAACG
GGAAACGTCGAGGCCGCGATTAAATTCCAACATGGATGCTGATTTATATGGGTATA
AATGGGCTCGCGATAATGTCGGGCAATCAGGTGCGACAATCTATCGCTTGTATGGG
AAGCCCGATGCGCCAGAGTTGTTTCTGAAACATGGCAAAGGTAGCGTTGCCAATGA
TGTTACAGATGAGATGGTCAGACTAAACTGGCTGACGGAATTTATGCCTCTTCCGAC
CATCAAGCATTTTATCCGTACTCCTGATGATGCATGGTTACTCACCACTGCGATCCC
CGGAAAAACAGCATTCCAGGTATTAGAAGAATATCCTGATTCAGGTGAAAATATTG
TTGATGCGCTGGCAGTGTTCCTGCGCCGGTTGCATTCGATTCCTGTTTGTAATTGTCC
TTTTAACAGCGATCGCGTATTTCGTCTCGCTCAGGCGCAATCACGAATGAATAACGG
TTTGGTTGATGCGAGTGATTTTGATGACGAGCGTAATGGCTGGCCTGTTGAACAAGT
CTGGAAAGAAATGCATAAACTTTTGCCATTCTCACCGGATTCAGTCGTCACTCATGG
TGATTTCTCACTTGATAACCTTATTTTTGACGAGGGGAAATTAATAGGTTGTATTGA
TGTTGGACGAGTCGGAATCGCAGACCGATACCAGGATCTTGCCATCCTATGGAACT
GCCTCGGTGAGTTTTCTCCTTCATTACAGAAACGGCTTTTTCAAAAATATGGTATTG
ATAATCCTGATATGAATAAATTGCAGTTTCATTTGATGCTCGATGAGTTTTTCTAActg
tcagaccaagtttactcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttgagatcctttttttctgcgcgta
atctgctgcttgcaaacaaaaaaaccaccgctaccagcggtggtttgtttgccggatcaagagctaccaactctttttccgaaggtaactggcttc
agcagagcgcagataccaaatactgtTcttctagtgtagccgtagttaggccaccacttcaagaactctgtagcaccgcctacatacctcgct
ctgctaatcctgttaccagtggctgctgccagtggcgataagtcgtgtcttaccgggttggactcaagacgatagttaccggataaggcgca
gcggtcgggctgaacggggggttcgtgcacacagcccagcttggagcgaacgacctacaccgaactgagatacctacagcgtgagctat
gagaaagcgccacgcttcccgaagggagaaaggcggacaggtatccggtaagcggcagggtcggaacaggagagcgcacgaggga
gcttccagggggaaacgcctggtatctttatagtcctgtcgggtttcgccacctctgacttgagcgtcgatttttgtgatgctcgtcagggggg
cggagcctatggaaaaacgccagcaacgcggcctttttacggttcctggccttttgctgGCCttttgctcacatgttctttcctgcgttatccc
ctgattctgtggataaccgtattaccgcctttgagtgagctgataccgctcgccgcagccgaacgaccgagcgcagcgagtcagtgagcg
aggaagcggaagagcgcccaatacgcaaaccgcctctccccgcgcgttggccgattcattaatgcagctggcacgacaggtttcccgact
ggaaagcgggcagtgagcgcaacgcaattaatgtgagttagctcactcattaggcaccccaggctttacactttatgcttccggctcgtatgt
tgtgtggaattgtgagcggataacaatttcacacaggaaacagctatgaccatgattacgccagatttaattaagg (SEQ ID NO. 3)
pACM-Beta

| | | |
|---|---|---|
| 5′-ITR | 1..141 | 141 |
| mCMV | 222..594 | 373 |
| CMV enhancer | 222..525 | 304 |
| minCMV promoter | 526..594 | 69 |
| Beta Spike Stabilized | 615..4427 | 3813 |
| SP | 615..653 | 39 |
| S1 | 654..2657 | 2004 |
| L18F | 666..668 | 3 |
| D80A | 852..854 | 3 |
| D215G | 1257..1259 | 3 |
| L242-L244 del | 1337^{∧}1338 | 0 |
| R246I | 1341..1343 | 3 |
| RBD | 1563..2228 | 666 |
| K417N | 1854..1856 | 3 |
| E484K | 2055..2057 | 3 |
| N501Y | 2106..2108 | 3 |
| D614G | 2445..2447 | 3 |
| Furin Recognition Site | 2650..2657 | 8 |
| A701V | 2706..2708 | 3 |
| Proline substitution | 3566..3566 | 1 |
| poly(A) signal | 4437..4485 | 49 |
| 3′-ITR | 4573..4702 | 130 |
| KanR | 5466..6275 | 810 |

cCTTAATTAGGCtgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatgattaacccgccatgctacttat
ctacgtagccatgctctaggaagatcggaattcgcccttaagCTAGCcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatgATACAGCTCCAC
CGCACATGCCACCCTCCGGATATATTCGTCTCGAGCAAATCACTTGGCAGTCTAGCG
GCggccgcctagatgccaccaTGTTCGTCTTCCTGGTCCTGCTGCCTCTGGTCTCCTCACAGTG
CGTCAATtTcACAACTCGGACTCAGCTGCCACCTGCTTATACTAATAGCTTCACCAGA
GGCGTGTACTATCCTGACAAGGTGTTTAGAAGCTCCGTGCTGCACTCTACACAGGAT
CTGTTTCTGCCATTCTTTAGCAACGTGACCTGGTTCCACGCCATCCACGTGAGCGGC
ACCAATGGCACAAAGCGGTTCGcCAATCCCGTGCTGCCTTTTAACGATGGCGTGTAC
TTCGCCTCTACCGAGAAGAGCAACATCATCAGAGGCTGGATCTTTGGCACCACACT
GGACTCCAAGACACAGTCTCTGCTGATCGTGAACAATGCCACCAACGTGGTCATCA
AGGTGTGCGAGTTCCAGTTTTGTAATGATCCCTTCCTGGGCGTGTACTATCACAAGA
ACAATAAGAGCTGGATGGAGTCCGAGTTTAGAGTGTATTCTAGCGCCAACAACTGC
ACATTTGAGTACGTGAGCCAGCCTTTCCTGATGGACCTGGAGGGCAAGCAGGGCAA
TTTCAAGAACCTGAGGGAGTTCGTGTTTAAGAATATCGACGGCTACTTCAAAATCTA
CTCTAAGCACACCCCCATCAACCTGGTGCGCGgCCTGCCTCAGGGCTTCAGCGCCCT
GGAGCCCCTGGTGGATCTGCCTATCGGCATCAACATCACCCGGTTTCAGACACTGCA
CAtAAGCTACCTGACACCCGGCGACTCCTCTAGCGGATGGACCGCCGGCGCTGCCGC
CTACTATGTGGGCTACCTCCAGCCCCGGACCTTCCTGCTGAAGTACAACGAGAATG
GCACCATCACAGACGCAGTGGATTGCGCCCTGGACCCCCTGAGCGAGACAAAGTGT
ACACTGAAGTCCTTTACCGTGGAGAAGGGCATCTATCAGACATCCAATTTCAGGGT
GCAGCCAACCGAGTCTATCGTGCGCTTTCCTAATATCACAAACCTGTGCCCATTTGG
CGAGGTGTTCAACGCAACCCGCTTCGCCAGCGTGTACGCCTGGAATAGGAAGCGGA
TCAGCAACTGCGTGGCCGACTATAGCGTGCTGTACAACTCCGCCTCTTTCAGCACCT
TTAAGTGCTATGGCGTGTCCCCCACAAAGCTGAATGACCTGTGCTTTACCAACGTCT
ACGCCGATTCTTTCGTGATCAGGGGCGACGAGGTGCGCCAGATCGCCCCCGGCCAG
ACAGGCAAcATCGCAGACTACAATTATAAGCTGCCAGACGATTTCACCGGCTGCGT
GATCGCCTGGAACAGCAACAATCTGGATTCCAAAGTGGGCGGCAACTACAATTATC
TGTACCGGCTGTTTAGAAAGAGCAATCTGAAGCCCTTCGAGAGGGACATCTCTACA
GAAATCTACCAGGCCGGCAGCACCCCTTGCAATGGCGTGaAGGGCTTTAACTGTTAT
TTCCCACTCCAGTCCTACGGCTTCCAGCCCACAtACGGCGTGGGCTATCAGCCTTACC
GCGTGGTGGTGCTGAGCTTTGAGCTGCTGCACGCCCCAGCAACAGTGTGCGGCCCC
AAGAAGTCCACCAATCTGGTGAAGAACAAGTGCGTGAACTTCAACTTCAACGGCCT
GACCGGCACAGGCGTGCTGACCGAGTCCAACAAGAAGTTCCTGCCATTTCAGCAGT
TCGGCAGGGACATCGCAGATACCACAGACGCCGTGCGCGACCCACAGACCCTGGAG
ATCCTGGACATCACACCCTGCTCTTTCGGCGGCGTGAGCGTGATCACACCCGGCACC
AATACAAGCAACCAGGTGgccgtgctgtatcagggggtgaattgtaccgaggTGCCCGTGGCTATCCAC
GCCGATCAGCTGACCCCAACATGGCGGGTGTACAGCACCGGCTCCAACGTCTTCCA
GACAAGAGCCGGATGCCTGATCGGAGCAGAGCACGTGAACAATTCCTATGAGTGCG
ACATCCCAATCGGCGCCGGCATCTGTGCCTCTTACCAGACCcagacaaactctcccggaagcgcc
agtagcgtggcctcccagTCTATCATCGCCTATACCATGTCCCTGGGCGtCGAGAACAGCGTGG
CCTACTCTAACAATAGCATCGCCATCCCAACCAACTTCACAATCTCTGTGACCACAG
AGATCCTGCCCGTGTCCATGACCAAGACATCTGTGGACTGCACAATGTATATCTGTG
GCGATTCTACCGAGTGCAGCAACCTGCTGCTCCAGTACGGCAGCTTTTGTACCCAGC
TGAATAGAGCCCTGACAGGCATCGCCGTGGAGCAGGATAAGAACACACAGGAGGT
GTTCGCCCAGGTGAAGCAAATCTACAAGACCCCCCCTATCAAGGACTTTGGCGGCT
TCAATTTTTCCCAGATCCTGCCTGATCCATCCAAGCCTTCTAAGCGGAGCTTTATCG
AGGACCTGCTGTTCAACAAGGTGACCCTGGCCGATGCCGGCTTCATCAAGCAGTAT
GGCGATTGCCTGGGCGACATCGCAGCCAGGGACCTGATCTGCGCCCAGAAGTTTAA
TGGCCTGACCGTGCTGCCACCCCTGCTGACAGATGAGATGATCGCACAGTACACAA
GCGCCCTGCTGGCCGGCACCATCACATCCGGATGGACCTTCGGCGCAGGAGCCGCC
CTCCAGATCCCCTTTGCCATGCAGATGGCCTATAGGTTCAACGGCATCGGCGTGACC
CAGAATGTGCTGTACGAGAACCAGAAGCTGATCGCCAATCAGTTTAACTCCGCCAT
CGGCAAGATCCAGGACAGCCTGTCCTCTACAGCCAGCGCCCTGGGCAAGCTCCAGG
ATGTGGTGAATCAGAACGCCCAGGCCCTGAATACCCTGGTGAAGCAGCTGAGCAGC
AACTTCGGCGCCATCTCTAGCGTGCTGAATGAcatcctgagccggctggacccaccggaggcagaggtgc
agatcGACCGGCTGATCACCGGCCGGCTCCAGAGCCTCCAGACCTATGTGACACAGCA
GCTGATCAGGGCCGCCGAGATCAGGGCCAGCGCCAATCTGGCAGCAACCAAGATGT
CCGAGTGCGTGCTGGGCCAGTCTAAGAGAGTGGACTTTTGTGGCAAGGGCTATCAC
CTGATGTCCTTCCCTCAGTCTGCCCCACACGGCGTGGTGTTTCTGCACGTGACCTAC
GTGCCCGCCCAGGAGAAGAACTTCACCACAGCCCCTGCCATCTGCCACGATGGCAA
GGCCCACTTTCCAAGGGAGGGCGTGTTCGTGTCCAACGGCACCCACTGGTTTGTGAC
ACAGCGCAATTTCTACGAGCCCCAGATCATCACCACAGACAACACCTTCGTGAGCG
GCAACTGTGACGTGGTCATCGGCATCGTGAACAATACCGTGTATGATCCACTCCAG
CCCGAGCTGGACAGCTTTAAGGAGGAGCTGGATAAGTATTTCAAGAATCACACCTC
CCCTGACGTGGATCTGGGCGACATCAGCGGCATCAATGCCTCCGTGGTGAACATCC
AGAAGGAGATCGACCGCCTGAACGAGGTGGCTAAGAATCTGAACGAGAGCCTGAT
CGACCTCCAGGAGCTGGGCAAGTATGAGCAGTACATCAAGTGGCCCTGGTACATCT
GGCTGGGCTTCATCGCCGGCCTGATCGCCATCGTGATGGTGACCATCATGCTGTGCT
GTATGACATCCTGCTGTTCTTGCCTGAAGGGCTGCTGTAGCTGTGGCTCCTGCTGTA
AGTTTGACGAGGATGACTCTGAACCTGTGCTGAAGGGCGTGAAGCTGCACTACACC
TAAATCGGATCTAATAAAATATCTTTATTTTCATTACATCTGTGTGTTGGTTTTTTGT
GTGCtcgagttaagggcgaattcccgataaggatcttcctagagcatggctacgtagataagtagcatggcgggttaatcattaactaca
aggaacccctagtgatggagttggccactccctctctgcgcgctcgctcgctcactgaggccgggcgaccaaaggtcgcccgacgcccg
ggctttgcccgggcggcctcagtgagcgagcgagcgcgcagccttaattaacctaattcactggccgtcgttttacaacgtcgtgactggg
aaaaccctggcgttacccaacttaatcgccttgcagcacatccccctttcgccagctggcgtaatagcgaagaggcccgcaccgatcgccc
ttcccaacagttgcgcagcctgaatggcgaatgggacgcgccctgtagcggcgcattaagcgcggcgggtgtggtggttacgcgcagcg
tgaccgctacacttgccagcgccctagcgcccgctcctttcgctttcttcccttcctttctcgccacgttcgccggctttccccgtcaagctcta
aatcgggggctccctttagggttccgatttagtgctttacggcacctcgaccccaaaaaacttgattagggtgatggttcacgtagtgggcca
tcgccctgatagacggtttttcgccctttgacgttggagtccacgttctttaatagtggactcttgttccaaactggaacaacactcaaccctatc
tcggtctattcttttgatttataagggattttgccgatttcggcctattggttaaaaaatgagctgatttaacaaaaatttaacgcgaattttaac
aaaatattaacgtttataatttcaggtggcatctttcggggaaatgtgcgcggaacccctatttgtttatttttctaaatacattcaaatatgtat
ccgctcatgagacaataaccctgataaatgcttcaataatattgaaaaaggaagagtATGAGCCATATTCAACGGGAAACGT
CGAGGCCGCGATTAAATTCCAACATGGATGCTGATTTATATGGGTATAAATGGGCT
CGCGATAATGTCGGGCAATCAGGTGCGACAATCTATCGCTTGTATGGGAAGCCCGA
TGCGCCAGAGTTGTTTCTGAAACATGGCAAAGGTAGCGTTGCCAATGATGTTACAG
ATGAGATGGTCAGACTAAACTGGCTGACGGAATTTATGCCTCTTCCGACCATCAAG
CATTTTATCCGTACTCCTGATGATGCATGGTTACTCACCACTGCGATCCCCGGAAAA
ACAGCATTCCAGGTATTAGAAGAATATCCTGATTCAGGTGAAAATATTGTTGATGC
GCTGGCAGTGTTCCTGCGCCGGTTGCATTCGATTCCTGTTTGTAATTGTCCTTTTAAC
AGCGATCGCGTATTTCGTCTCGCTCAGGCGCAATCACGAATGAATAACGGTTTGGTT
GATGCGAGTGATTTTGATGACGAGCGTAATGGCTGGCCTGTTGAACAAGTCTGGAA
AGAAATGCATAAACTTTTGCCATTCTCACCGGATTCAGTCGTCACTCATGGTGATTT
CTCACTTGATAACCTTATTTTTGACGAGGGGAAATTAATAGGTTGTATTGATGTTGG
ACGAGTCGGAATCGCAGACCGATACCAGGATCTTGCCATCCTATGGAACTGCCTCG
GTGAGTTTTCTCCTTCATTACAGAAACGGCTTTTTCAAAAATATGGTATTGATAATC
CTGATATGAATAAATTGCAGTTTCATTTGATGCTCGATGAGTTTTTCTAActgtcagaccaa
gtttactcatatatactttagattgatttaaaacttcatttttaatttaaaaggatctaggtgaagatcctttttgataatctcatgaccaaaatc
ccttaacgtgagttttcgttccactgagcgtcagaccccgtagaaaagatcaaaggatcttcttgagatcctttttttctgcgcgtaatctgctgc
ttgcaaacaaaaaaaccaccgctaccagcggtggtttgtttgccggatcaagagctaccaactctttttccgaaggtaactggcttcagcagagcg
cagataccaaatactgtccttctagtgtagccgtagttaggccaccacttcaagaactctgtagcaccgcctacatacctcgctctgctaatcct
gttaccagtggctgctgccagtggcgataagtcgtgtcttaccgggttggactcaagacgatagttaccggataaggcgcagcggtcggg
ctgaacggggggttcgtgcacacagcccagcttggagcgaacgacctacaccgaactgagatacctacagcgtgagctatgagaaagc
gccacgcttcccgaagggagaaaggcggacaggtatccggtaagcggcagggtcggaacaggagagcgcacgagggagcttccagg
gggaaacgcctggtatctttatagtcctgtcgggtttcgccacctctgacttgagcgtcgatttttgtgatgctcgtcaggggggcggagccta
tggaaaaacgccagcaacgcggcctttttacggttcctggccttttgctgcggttttgctcacatgttctttcctgcgttatcccctgattctgtg
gataaccgtattaccgcctttgagtgagctgataccgctcgccgcagccgaacgaccgagcgcagcgagtcagtgagcgaggaagcgga
agagcgcccaatacgcaaaccgcctctccccgcgcgttggccgattcattaatgcagctggcacgacaggtttcccgactggaaagcgg
gcagtgagcgcaacgcaattaatgtgagttagctcactcattaggcaccccaggctttacactttatgcttccggctcgtatgttgtgtggaatt
gtgagcggataacaatttcacacaggaaacagctatgaccatgattacgccagatttaattaagg (SEQ ID NO: 4)
pACM-Delta

| | | |
|---|---|---|
| 5′-ITR | 1..141 | 141 |
| mCMV | 222..594 | 373 |
| CMV enhancer | 222..525 | 304 |
| minCMV promoter | 526..594 | 69 |
| Delta Spike Stabilized | 615..4430 | 3816 |
| SP | 615..653 | 39 |
| S1 | 654..2660 | 2007 |
| T19R | 669..671 | 3 |
| G142D | 1038..1040 | 3 |
| E156G | 1080..1082 | 3 |
| FR 156-157 del | 1082^{∧}1083 | 0 |
| RBD | 1566..2231 | 666 |
| K417N | 1857..1859 | 3 |
| L452R | 1962..1964 | 3 |
| T478K | 2040..2042 | 3 |
| D614G | 2448..2450 | 3 |
| P681R | 2649..2651 | 3 |
| Furin Recognition Site | 2653..2660 | 8 |
| D950N | 3456..3458 | 3 |
| Proline substitution | 3569..3569 | 1 |
| poly(A) signal | 4440..4488 | 49 |
| 3′-ITR | 4576..4705 | 130 |
| KanR | 5469..6278 | 810 |

cCTTAATTAGGCtgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatgattaacccgccatgctacttat
ctacgtagccatgctctaggaagatcggaattcgcccttaagCTAGCcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatgATACAGCTCCAC
CGCACATGCCACCCTCCGGATATATTCGTCTCGAGCAAATCACTTGGCAGTCTAGCG
GCggccgcctagatgccaccaTGTTCGTCTTCCTGGTCCTGCTGCCTCTGGTCTCCTCACAGTG
CGTCAATCTGAgAACTCGGACTCAGCTGCCACCTGCTTATACTAATAGCTTCACCAG
AGGCGTGTACTATCCTGACAAGGTGTTTAGAAGCTCCGTGCTGCACTCTACACAGG
ATCTGTTTCTGCCATTCTTTAGCAACGTGACCTGGTTCCACGCCATCCACGTGAGCG
GCACCAATGGCACAAAGCGGTTCGACAATCCCGTGCTGCCTTTTAACGATGGCGTG
TACTTCGCCTCTACCGAGAAGAGCAACATCATCAGAGGCTGGATCTTTGGCACCAC
ACTGGACTCCAAGACACAGTCTCTGCTGATCGTGAACAATGCCACCAACGTGGTCA
TCAAGGTGTGCGAGTTCCAGTTTTGTAATGATCCCTTCCTGGaCGTGTACTATCACAA
GAACAATAAGAGCTGGATGGAGTCCGgGGTGTATTCTAGCGCCAACAACTGCACAT
TTGAGTACGTGAGCCAGCCTTTCCTGATGGACCTGGAGGGCAAGCAGGGCAATTTC
AAGAACCTGAGGGAGTTCGTGTTTAAGAATATCGACGGCTACTTCAAAATCTACTCT
AAGCACACCCCCATCAACCTGGTGCGCGACCTGCCTCAGGGCTTCAGCGCCCTGGA
GCCCCTGGTGGATCTGCCTATCGGCATCAACATCACCCGGTTTCAGACACTGCTGGC
CCTGCACAGAAGCTACCTGACACCCGGCGACTCCTCTAGCGGATGGACCGCCGGCG
CTGCCGCCTACTATGTGGGCTACCTCCAGCCCCGGACCTTCCTGCTGAAGTACAACG
AGAATGGCACCATCACAGACGCAGTGGATTGCGCCCTGGACCCCCTGAGCGAGACA
AAGTGTACACTGAAGTCCTTTACCGTGGAGAAGGGCATCTATCAGACATCCAATTTC
AGGGTGCAGCCAACCGAGTCTATCGTGCGCTTTCCTAATATCACAAACCTGTGCCCA
TTTGGCGAGGTGTTCAACGCAACCCGCTTCGCCAGCGTGTACGCCTGGAATAGGAA
GCGGATCAGCAACTGCGTGGCCGACTATAGCGTGCTGTACAACTCCGCCTCTTTCAG
CACCTTTAAGTGCTATGGCGTGTCCCCCACAAAGCTGAATGACCTGTGCTTTACCAA
CGTCTACGCCGATTCTTTCGTGATCAGGGGCGACGAGGTGCGCCAGATCGCCCCCG
GCCAGACAGGCAAcATCGCAGACTACAATTATAAGCTGCCAGACGATTTCACCGGC
TGCGTGATCGCCTGGAACAGCAACAATCTGGATTCCAAAGTGGGCGGCAACTACAA
TTATCgGTACCGGCTGTTTAGAAAGAGCAATCTGAAGCCCTTCGAGAGGGACATCTC
TACAGAAATCTACCAGGCCGGCAGCAagCCTTGCAATGGCGTGGAGGGCTTTAACTG
TTATTTCCCACTCCAGTCCTACGGCTTCCAGCCCACAAACGGCGTGGGCTATCAGCC
TTACCGCGTGGTGGTGCTGAGCTTTGAGCTGCTGCACGCCCCAGCAACAGTGTGCG
GCCCCAAGAAGTCCACCAATCTGGTGAAGAACAAGTGCGTGAACTTCAACTTCAAC
GGCCTGACCGGCACAGGCGTGCTGACCGAGTCCAACAAGAAGTTCCTGCCATTTCA
GCAGTTCGGCAGGGACATCGCAGATACCACAGACGCCGTGCGCGACCCACAGACCC
TGGAGATCCTGGACATCACACCCTGCTCTTTCGGCGGCGTGAGCGTGATCACACCCG
GCACCAATACAAGCAACCAGGTGgccgtgctgtatcagggggtgaattgtaccgaggTGCCCGTGGCTA
TCCACGCCGATCAGCTGACCCCAACATGGCGGGTGTACAGCACCGGCTCCAACGTC
TTCCAGACAAGAGCCGGATGCCTGATCGGAGCAGAGCACGTGAACAATTCCTATGA
GTGCGACATCCCAATCGGCGCCGGCATCTGTGCCTCTTACCAGACCcagacaaactctcGcg
gaagcgccagtagcgtggcctcccagTCTATCATCGCCTATACCATGTCCCTGGGCGCCGAGAACA
GCGTGGCCTACTCTAACAATAGCATCGCCATCCCAACCAACTTCACAATCTCTGTGA
CCACAGAGATCCTGCCCGTGTCCATGACCAAGACATCTGTGGACTGCACAATGTAT
ATCTGTGGCGATTCTACCGAGTGCAGCAACCTGCTGCTCCAGTACGGCAGCTTTTGT
ACCCAGCTGAATAGAGCCCTGACAGGCATCGCCGTGGAGCAGGATAAGAACACAC
AGGAGGTGTTCGCCCAGGTGAAGCAAATCTACAAGACCCCCCCTATCAAGGACTTT
GGCGGCTTCAATTTTTCCCAGATCCTGCCTGATCCATCCAAGCCTTCTAAGCGGAGC
TTTATCGAGGACCTGCTGTTCAACAAGGTGACCCTGGCCGATGCCGGCTTCATCAAG
CAGTATGGCGATTGCCTGGGCGACATCGCAGCCAGGGACCTGATCTGCGCCCAGAA
GTTTAATGGCCTGACCGTGCTGCCACCCCTGCTGACAGATGAGATGATCGCACAGT
ACACAAGCGCCCTGCTGGCCGGCACCATCACATCCGGATGGACCTTCGGCGCAGGA
GCCGCCCTCCAGATCCCCTTTGCCATGCAGATGGCCTATAGGTTCAACGGCATCGGC
GTGACCCAGAATGTGCTGTACGAGAACCAGAAGCTGATCGCCAATCAGTTTAACTC
CGCCATCGGCAAGATCCAGGACAGCCTGTCCTCTACAGCCAGCGCCCTGGGCAAGC
TCCAGaATGTGGTGAATCAGAACGCCCAGGCCCTGAATACCCTGGTGAAGCAGCTG
AGCAGCAACTTCGGCGCCATCTCTAGCGTGCTGAATGAcatcctgagccggctggacccaccggag
gcagaggtgcagatcGACCGGCTGATCACCGGCCGGCTCCAGAGCCTCCAGACCTATGTGA
CACAGCAGCTGATCAGGGCCGCCGAGATCAGGGCCAGCGCCAATCTGGCAGCAACC
AAGATGTCCGAGTGCGTGCTGGGCCAGTCTAAGAGAGTGGACTTTTGTGGCAAGGG
CTATCACCTGATGTCCTTCCCTCAGTCTGCCCCACACGGCGTGGTGTTTCTGCACGT
GACCTACGTGCCCGCCCAGGAGAAGAACTTCACCACAGCCCCTGCCATCTGCCACG
ATGGCAAGGCCCACTTTCCAAGGGAGGGCGTGTTCGTGTCCAACGGCACCCACTGG
TTTGTGACACAGCGCAATTTCTACGAGCCCCAGATCATCACCACAGACAACACCTTC
GTGAGCGGCAACTGTGACGTGGTCATCGGCATCGTGAACAATACCGTGTATGATCC
ACTCCAGCCCGAGCTGGACAGCTTTAAGGAGGAGCTGGATAAGTATTTCAAGAATC
ACACCTCCCCTGACGTGGATCTGGGCGACATCAGCGGCATCAATGCCTCCGTGGTG
AACATCCAGAAGGAGATCGACCGCCTGAACGAGGTGGCTAAGAATCTGAACGAGA
GCCTGATCGACCTCCAGGAGCTGGGCAAGTATGAGCAGTACATCAAGTGGCCCTGG
TACATCTGGCTGGGCTTCATCGCCGGCCTGATCGCCATCGTGATGGTGACCATCATG
CTGTGCTGTATGACATCCTGCTGTTCTTGCCTGAAGGGCTGCTGTAGCTGTGGCTCC
TGCTGTAAGTTTGACGAGGATGACTCTGAACCTGTGCTGAAGGGCGTGAAGCTGCA
CTACACCTAAATCGGATCTAATAAAATATCTTTATTTTCATTACATCTGTGTGTTGGT
TTTTTGTGTGCtcgagttaagggcgaattcccgataaggatcttcctagagcatggctacgtagataagtagcatgggggttaat
cattaactacaaggaacccctagtgatggagttggccactccctctctgcgcgctcgctcgctcactgaggccgggcgaccaaaggtcgc
ccgacgcccgggctttgcccgggcggcctcagtgagcgagcgagcgcgcagccttaattaacctaattcactggccgtcgttttacaacgt
cgtgactgggaaaaccctggcgttacccaacttaatcgccttgcagcacatccccctttcgccagctggcgtaatagcgaagaggcccgca
ccgatcgcccttcccaacagttgcgcagcctgaatggcgaatgggacgcgccctgtagcggcgcattaagcgcggcgggtgtggtggtt
acgcgcagcgtgaccgctacacttgccagcgccctagcgcccgctcctttcgctttcttcccttcctttctcgccacgttcgccggctttcccc
gtcaagctctaaatcgggggctccctttagggttccgatttagtgctttacggcacctcgaccccaaaaaacttgattagggtgatggttcacg
tagtgggccatcgccctgatagacggtttttcgccctttgacgttggagtccacgttctttaatagtggactcttgttccaaactggaacaacac
tcaaccctatctcggtctattcttttgatttataagggattttgccgatttcggcctattggttaaaaaatgagctgatttaacaaaaatttaacg
cgaattttaacaaaatattaacgtttataatttcaggtggcatctttcggggaaatgtgcgcggaacccctatttgtttatttttctaaatacatt
caaatatgtatccgctcatgagacaataaccctgataaatgcttcaataatattgaaaaaggaagagtATGAGCCATATTCAACGG
GAAACGTCGAGGCCGCGATTAAATTCCAACATGGATGCTGATTTATATGGGTATAA
ATGGGCTCGCGATAATGTCGGGCAATCAGGTGCGACAATCTATCGCTTGTATGGGA
AGCCCGATGCGCCAGAGTTGTTTCTGAAACATGGCAAAGGTAGCGTTGCCAATGAT
GTTACAGATGAGATGGTCAGACTAAACTGGCTGACGGAATTTATGCCTCTTCCGACC
ATCAAGCATTTTATCCGTACTCCTGATGATGCATGGTTACTCACCACTGCGATCCCC
GGAAAAACAGCATTCCAGGTATTAGAAGAATATCCTGATTCAGGTGAAAATATTGT
TGATGCGCTGGCAGTGTTCCTGCGCCGGTTGCATTCGATTCCTGTTTGTAATTGTCCT
TTTAACAGCGATCGCGTATTTCGTCTCGCTCAGGCGCAATCACGAATGAATAACGGT
TTGGTTGATGCGAGTGATTTTGATGACGAGCGTAATGGCTGGCCTGTTGAACAAGTC
TGGAAAGAAATGCATAAACTTTTGCCATTCTCACCGGATTCAGTCGTCACTCATGGT
GATTTCTCACTTGATAACCTTATTTTTGACGAGGGGAAATTAATAGGTTGTATTGAT
GTTGGACGAGTCGGAATCGCAGACCGATACCAGGATCTTGCCATCCTATGGAACTG
CCTCGGTGAGTTTTCTCCTTCATTACAGAAACGGCTTTTTCAAAAATATGGTATTGA
TAATCCTGATATGAATAAATTGCAGTTTCATTTGATGCTCGATGAGTTTTTCTAActgtc
agaccaagtttactcatatatactttagattgatttaaaacttcatttttaatttaaaaggatctaggtgaagatcctttttgataatctcatgac
caaaatcccttaacgtgagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccgaaggtaactggcttcag
cagagcgcagataccaaatactgtccttctagtgtagccgtagttaggccaccacttcaagaactctgtagcaccgcctacatacctcgctctgc
taatcctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatgaga
aagcgccacgcttcccgaagggagaaaggcggacaggtatccggtaagcggcagggtcggaacaggagagcgcacgagggagcttc
cagggggaaacgcctggtatctttatagtcctgtcgggtttcgccacctctgacttgagcgtcgatttttgtgatgctcgtcagggggggga
gcctatggaaaaacgccagcaacgcggcctttttacggttcctggccttttgctgcggttttgctcacatgttctttcctgcgttatcccctgattc
tgtggataaccgtattaccgcctttgagtgagctgataccgctcgccgcagccgaacgaccgagcgcagcgagtcagtgagcgaggaag
cggaagagcgcccaatacgcaaaccgcctctccccgcgcgttggccgattcattaatgcagctggcacgacaggtttcccgactggaaag
cgggcagtgagcgcaacgcaattaatgtgagttagctcactcattaggcaccccaggctttacactttatgcttccggctcgtatgttgtgtgg
aattgtgagcggataacaatttcacacaggaaacagctatgaccatgattacgccagatttaattaagg (SEQ ID NO: 5)
pACM1

| | | |
|---|---|---|
| 5′-ITR | 1..141 | 141 |
| mCMV promoter | 222..594 | 373 |
| CMV enhancer | 222..525 | 304 |
| miniCMV promoter | 526..594 | 69 |
| Wuhan Spike Stabilized | 615..4436 | 3822 |
| SP | 615..653 | 39 |
| S1 | 654..2666 | 2013 |
| RBD | 1572..2237 | 666 |
| Furin Recognition Site | 2659..2666 | 8 |
| Proline substitution | 3575..3575 | 1 |
| poly(A) signal | 4446..4494 | 49 |
| 3′-ITR | 4582..4711 | 130 |
| KanR | 5475..6284 | 810 |

cCTTAATTAGGCtgcgcgctcgctcgctcactgaggccgcccgggcaaagcccgggcgtcgggcgacctttggtcgcccggc
ctcagtgagcgagcgagcgcgcagagagggagtggccaactccatcactaggggttccttgtagttaatgattaacccgccatgctacttat
ctacgtagccatgctctaggaagatcggaattcgcccttaagCTAGCcgttacataacttacggtaaatggcccgcctggctgaccgcc
caacgacccccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggcccgcctggc
attatgcccagtacatgaccttatgggactttcctacttggcagtacatctacgtattagtcatcgctattaccatgATACAGCTCCAC
CGCACATGCCACCCTCCGGATATATTCGTCTCGAGCAAATCACTTGGCAGTCTAGCG
GCggccgcctagatgccaccaTGTTCGTCTTCCTGGTCCTGCTGCCTCTGGTCTCCTCACAGTG
CGTCAATCTGACAACTCGGACTCAGCTGCCACCTGCTTATACTAATAGCTTCACCAG
AGGCGTGTACTATCCTGACAAGGTGTTTAGAAGCTCCGTGCTGCACTCTACACAGG
ATCTGTTTCTGCCATTCTTTAGCAACGTGACCTGGTTCCACGCCATCCACGTGAGCG
GCACCAATGGCACAAAGCGGTTCGACAATCCCGTGCTGCCTTTTAACGATGGCGTG
TACTTCGCCTCTACCGAGAAGAGCAACATCATCAGAGGCTGGATCTTTGGCACCAC
ACTGGACTCCAAGACACAGTCTCTGCTGATCGTGAACAATGCCACCAACGTGGTCA
TCAAGGTGTGCGAGTTCCAGTTTTGTAATGATCCCTTCCTGGGCGTGTACTATCACA
AGAACAATAAGAGCTGGATGGAGTCCGAGTTTAGAGTGTATTCTAGCGCCAACAAC
TGCACATTTGAGTACGTGAGCCAGCCTTTCCTGATGGACCTGGAGGGCAAGCAGGG
CAATTTCAAGAACCTGAGGGAGTTCGTGTTTAAGAATATCGACGGCTACTTCAAAA
TCTACTCTAAGCACACCCCCATCAACCTGGTGCGCGACCTGCCTCAGGGCTTCAGCG
CCCTGGAGCCCCTGGTGGATCTGCCTATCGGCATCAACATCACCCGGTTTCAGACAC
TGCTGGCCCTGCACAGAAGCTACCTGACACCCGGCGACTCCTCTAGCGGATGGACC
GCCGGCGCTGCCGCCTACTATGTGGGCTACCTCCAGCCCCGGACCTTCCTGCTGAAG
TACAACGAGAATGGCACCATCACAGACGCAGTGGATTGCGCCCTGGACCCCCTGAG
CGAGACAAAGTGTACACTGAAGTCCTTTACCGTGGAGAAGGGCATCTATCAGACAT
CCAATTTCAGGGTGCAGCCAACCGAGTCTATCGTGCGCTTTCCTAATATCACAAACC
TGTGCCCATTTGGCGAGGTGTTCAACGCAACCCGCTTCGCCAGCGTGTACGCCTGGA
ATAGGAAGCGGATCAGCAACTGCGTGGCCGACTATAGCGTGCTGTACAACTCCGCC
TCTTTCAGCACCTTTAAGTGCTATGGCGTGTCCCCCACAAAGCTGAATGACCTGTGC
TTTACCAACGTCTACGCCGATTCTTTCGTGATCAGGGGCGACGAGGTGCGCCAGATC
GCCCCCGGCCAGACAGGCAAGATCGCAGACTACAATTATAAGCTGCCAGACGATTT
CACCGGCTGCGTGATCGCCTGGAACAGCAACAATCTGGATTCCAAAGTGGGCGGCA
ACTACAATTATCTGTACCGGCTGTTTAGAAAGAGCAATCTGAAGCCCTTCGAGAGG
GACATCTCTACAGAAATCTACCAGGCCGGCAGCACCCCTTGCAATGGCGTGGAGGG
CTTTAACTGTTATTTCCCACTCCAGTCCTACGGCTTCCAGCCCACAAACGGCGTGGG
CTATCAGCCTTACCGCGTGGTGGTGCTGAGCTTTGAGCTGCTGCACGCCCCAGCAAC
AGTGTGCGGCCCCAAGAAGTCCACCAATCTGGTGAAGAACAAGTGCGTGAACTTCA
ACTTCAACGGCCTGACCGGCACAGGCGTGCTGACCGAGTCCAACAAGAAGTTCCTG
CCATTTCAGCAGTTCGGCAGGGACATCGCAGATACCACAGACGCCGTGCGCGACCC
ACAGACCCTGGAGATCCTGGACATCACACCCTGCTCTTTCGGCGGCGTGAGCGTGA
TCACACCCGGCACCAATACAAGCAACCAGGTGGCCGTGCTGTATCAGGACGTGAAT
TGTACCGAGGTGCCCGTGGCTATCCACGCCGATCAGCTGACCCCAACATGGCGGGT
GTACAGCACCGGCTCCAACGTCTTCCAGACAAGAGCCGGATGCCTGATCGGAGCAG
AGCACGTGAACAATTCCTATGAGTGCGACATCCCAATCGGCGCCGGCATCTGTGCC
TCTTACCAGACCcagacaaactctcccggaagcgccagtagcgtggcctcccagTCTATCATCGCCTATACC
ATGTCCCTGGGCGCCGAGAACAGCGTGGCCTACTCTAACAATAGCATCGCCATCCC
AACCAACTTCACAATCTCTGTGACCACAGAGATCCTGCCCGTGTCCATGACCAAGA
CATCTGTGGACTGCACAATGTATATCTGTGGCGATTCTACCGAGTGCAGCAACCTGC
TGCTCCAGTACGGCAGCTTTTGTACCCAGCTGAATAGAGCCCTGACAGGCATCGCC
GTGGAGCAGGATAAGAACACACAGGAGGTGTTCGCCCAGGTGAAGCAAATCTACA
AGACCCCCCCTATCAAGGACTTTGGCGGCTTCAATTTTTCCCAGATCCTGCCTGATC
CATCCAAGCCTTCTAAGCGGAGCTTTATCGAGGACCTGCTGTTCAACAAGGTGACCC
TGGCCGATGCCGGCTTCATCAAGCAGTATGGCGATTGCCTGGGCGACATCGCAGCC
AGGGACCTGATCTGCGCCCAGAAGTTTAATGGCCTGACCGTGCTGCCACCCCTGCTG
ACAGATGAGATGATCGCACAGTACACAAGCGCCCTGCTGGCCGGCACCATCACATC
CGGATGGACCTTCGGCGCAGGAGCCGCCCTCCAGATCCCCTTTGCCATGCAGATGG
CCTATAGGTTCAACGGCATCGGCGTGACCCAGAATGTGCTGTACGAGAACCAGAAG
CTGATCGCCAATCAGTTTAACTCCGCCATCGGCAAGATCCAGGACAGCCTGTCCTCT
ACAGCCAGCGCCCTGGGCAAGCTCCAGGATGTGGTGAATCAGAACGCCCAGGCCCT
GAATACCCTGGTGAAGCAGCTGAGCAGCAACTTCGGCGCCATCTCTAGCGTGCTGA
ATGAcatcctgagccggctggacccaccggaggcagaggtgcagatcGACCGGCTGATCACCGGCCGGCTC
CAGAGCCTCCAGACCTATGTGACACAGCAGCTGATCAGGGCCGCCGAGATCAGGGC
CAGCGCCAATCTGGCAGCAACCAAGATGTCCGAGTGCGTGCTGGGCCAGTCTAAGA
GAGTGGACTTTTGTGGCAAGGGCTATCACCTGATGTCCTTCCCTCAGTCTGCCCCAC
ACGGCGTGGTGTTTCTGCACGTGACCTACGTGCCCGCCCAGGAGAAGAACTTCACC
ACAGCCCCTGCCATCTGCCACGATGGCAAGGCCCACTTTCCAAGGGAGGGCGTGTT
CGTGTCCAACGGCACCCACTGGTTTGTGACACAGCGCAATTTCTACGAGCCCCAGAT
CATCACCACAGACAACACCTTCGTGAGCGGCAACTGTGACGTGGTCATCGGCATCG
TGAACAATACCGTGTATGATCCACTCCAGCCCGAGCTGGACAGCTTTAAGGAGGAG
CTGGATAAGTATTTCAAGAATCACACCTCCCCTGACGTGGATCTGGGCGACATCAG
CGGCATCAATGCCTCCGTGGTGAACATCCAGAAGGAGATCGACCGCCTGAACGAGG
TGGCTAAGAATCTGAACGAGAGCCTGATCGACCTCCAGGAGCTGGGCAAGTATGAG
CAGTACATCAAGTGGCCCTGGTACATCTGGCTGGGCTTCATCGCCGGCCTGATCGCC
ATCGTGATGGTGACCATCATGCTGTGCTGTATGACATCCTGCTGTTCTTGCCTGAAG
GGCTGCTGTAGCTGTGGCTCCTGCTGTAAGTTTGACGAGGATGACTCTGAACCTGTG
CTGAAGGGCGTGAAGCTGCACTACACCTAAATCGGATCTAATAAAATATCTTTATTT
TCATTACATCTGTGTGTTGGTTTTTTGTGTGCtcgagttaagggcgaattcccgataaggatcttcctagagca
tggctacgtagataagtagcatggcgggttaatcattaactacaaggaacccctagtgatggagttggccactccctctctgcgcgctcgctc
gctcactgaggccgggcgaccaaaggtcgcccgacgcccgggctttgcccgggcggcctcagtgagcgagcgagcgcgcagccttaa
ttaacctaattcactggccgtcgttttacaacgtcgtgactgggaaaaccctggcgttacccaacttaatcgccttgcagcacatccccctttcg
ccagctggcgtaatagcgaagaggcccgcaccgatcgcccttcccaacagttgcgcagcctgaatggcgaatgggacgcgccctgtag
cggcgcattaagcgcggcgggtgtggtggttacgcgcagcgtgaccgctacacttgccagcgccctagcgcccgctcctttcgctttcttc
ccttcctttctcgccacgttcgccggctttccccgtcaagctctaaatcgggggctccctttagggttccgatttagtgctttacggcacctcga
ccccaaaaaacttgattagggtgatggttcacgtagtgggccatcgccctgatagacggtttttcgccctttgacgttggagtccacgttcttta
atagtggactcttgttccaaactggaacaacactcaaccctatctcggtctattcttttgatttataagggattttgccgatttcggcctattggt
taaaaaatgagctgatttaacaaaaatttaacgcgaattttaacaaaatattaacgtttataatttcaggtggcatctttcggggaaatgtgcgcg
gaacccctatttgtttatttttctaaatacattcaaatatgtatccgctcatgagacaataaccctgataaatgcttcaataatattgaaaaagga
agagtATGAGCCATATTCAACGGGAAACGTCGAGGCCGCGATTAAATTCCAACATGGATG
CTGATTTATATGGGTATAAATGGGCTCGCGATAATGTCGGGCAATCAGGTGCGACA
ATCTATCGCTTGTATGGGAAGCCCGATGCGCCAGAGTTGTTTCTGAAACATGGCAA
AGGTAGCGTTGCCAATGATGTTACAGATGAGATGGTCAGACTAAACTGGCTGACGG
AATTTATGCCTCTTCCGACCATCAAGCATTTTATCCGTACTCCTGATGATGCATGGTT
ACTCACCACTGCGATCCCCGGAAAAACAGCATTCCAGGTATTAGAAGAATATCCTG
ATTCAGGTGAAAATATTGTTGATGCGCTGGCAGTGTTCCTGCGCCGGTTGCATTCGA
TTCCTGTTTGTAATTGTCCTTTTAACAGCGATCGCGTATTTCGTCTCGCTCAGGCGCA
ATCACGAATGAATAACGGTTTGGTTGATGCGAGTGATTTTGATGACGAGCGTAATG
GCTGGCCTGTTGAACAAGTCTGGAAAGAAATGCATAAACTTTTGCCATTCTCACCGG
ATTCAGTCGTCACTCATGGTGATTTCTCACTTGATAACCTTATTTTTGACGAGGGGA
AATTAATAGGTTGTATTGATGTTGGACGAGTCGGAATCGCAGACCGATACCAGGAT
CTTGCCATCCTATGGAACTGCCTCGGTGAGTTTTCTCCTTCATTACAGAAACGGCTTT
TTCAAAAATATGGTATTGATAATCCTGATATGAATAAATTGCAGTTTCATTTGATGC
TCGATGAGTTTTTCTAActgtcagaccaagtttactcatatatactttagattgatttaaaacttcatttttaatttaaaaggatcta
ggtgaagatcctttttgataatctcatgaccaaaatcccttaacgtgagttttcgttccactgagcgtcagaccccgtagaaaagatcaaagga
tcttcttgagatcctttttttctgcgcgtaatctgctgcttgcaaacaaaaaaaccaccgctaccagcggtggtttgtttgccggatcaagagcta
ccaactctttttccgaaggtaactggcttcagcagagcgcagataccaaatactgtccttctagtgtagccgtagttaggccaccacttcaaga
actctgtagcaccgcctacatacctcgctctgctaatcctgttaccagtggctgctgccagtggcgataagtcgtgtcttaccgggttggactc
aagacgatagttaccggataaggcgcagcggtcgggctgaacggggggttcgtgcacacagcccagcttggagcgaacgacctacacc
gaactgagatacctacagcgtgagctatgagaaagcgccacgcttcccgaagggagaaaggcggacaggtatccggtaagcggcagg
gtcggaacaggagagcgcacgagggagcttccagggggaaacgcctggtatctttatagtcctgtcgggtttcgccacctctgacttgagc
gtcgatttttgtgatgctcgtcaggggggcggagcctatggaaaaacgccagcaacgcggcctttttacggttcctggccttttgctgcggttt
tgctcacatgttctttcctgcgttatcccctgattctgtggataaccgtattaccgcctttgagtgagctgataccgctcgccgcagccgaacga
ccgagcgcagcgagtcagtgagcgaggaagcggaagagcgcccaatacgcaaaccgcctctccccgcgcgttggccgattcattaatg
cagctggcacgacaggtttcccgactggaaagcgggcagtgagcgcaacgcaattaatgtgagttagctcactcattaggcaccccaggc
tttacactttatgcttccggctcgtatgttgtgtggaattgtgagcggataacaatttcacacaggaaacagctatgaccatgattacgccagatt
taattaagg (SEQ ID NO: 6)

### REFERENCES

1. Baden L R, El Sahly H M, Essink B, Kotloff K, Frey S, Novak R, Diemert D, Spector S A, Rouphael N, Creech C B, et al. Efficacy and Safety of the mRNA-1273 SARS-COV-2 Vaccine. N Engl J Med. 2021; 384 (5): 403-16.
2. Polack F P, Thomas S J, Kitchin N, Absalon J, Gurtman A, Lockhart S, Perez J L, Perez Marc G, Moreira E D, Zerbini C, et al. Safety and Efficacy of the BNT162b2 mRNA Covid-19 Vaccine. N Engl J Med. 2020; 383 (27): 2603-15.
3. Heath P T, Galiza E P, Baxter D N, Boffito M, Browne D, Burns F, Chadwick D R, Clark R, Cosgrove C, Galloway J, et al. Safety and Efficacy of NVX-CoV2373 Covid-19 Vaccine. N Engl J Med. 2021; 385 (13): 1172-83.
4. Sadoff J, Gray G, Vandebosch A, Cardenas V, Shukarev G, Grinsztejn B, Goepfert P A. Truyers C, Fennema H, Spiessens B, et al. Safety and Efficacy of Single-Dose Ad26.COV2.S Vaccine against Covid-19. N Engl J Med. 2021; 384 (23): 2187-201.
5. Korber B. Fischer W M, Gnanakaran S, Yoon H, Theiler J, Abfalterer W, Hengartner N, Giorgi E E, Bhattacharya T, Foley B, et al. Tracking Changes in SARS-COV-2 Spike: Evidence that D614G Increases Infectivity of the COVID-19 Virus. Cell. 2020; 182 (4): 812-27 c19.
6. Garcia-Beltran W F, Lam E C, Astudillo M G, Yang D, Miller T E, Feldman J, Hauser B M, Caradonna™, Clayton K L, Nitido A D, et al. COVID-19-neutralizing antibodies predict disease severity and survival. Cell. 2021; 184 (2): 476-88 e11.
7. Legros V. Denolly S, Vogrig M, Boson B, Siret E, Rigaill J, Pillet S, Grattard F. Gonzalo S, Verhoeven P, et al. A longitudinal study of SARS-COV-2-infected patients reveals a high correlation between neutralizing antibodies and COVID-19 severity. Cellular & molecular immunology. 2021; 18 (2): 318-27.
8. Volz E, Mishra S, Chand M, Barrett J C, Johnson R, Geidelberg L, Hinsley W R, Laydon D J, Dabrera G, O'Toole A, et al. Assessing transmissibility of SARS-COV-2 lineage B.1.1.7 in England. Nature. 2021; 593 (7858): 266-9.
9. Tegally H. Wilkinson E, Giovanetti M, Iranzadeh A, Fonseca V. Giandhari J, Doolabh D. Pillay S, San E J, Msomi N, et al. Detection of a SARS-COV-2 variant of concern in South Africa. Nature. 2021; 592 (7854): 438-43.
10. Faria N R, Mellan T A, Whittaker C. Claro I M, Candido D D S, Mishra S, Crispim M A E, Sales F C S, Hawryluk I, McCrone J T, et al. Genomics and epidemiology of the P.1 SARS-COV-2 lineage in Manaus, Brazil. Science. 2021; 372 (6544): 815-21.
11. Naveca F G, Nascimento V, de Souza V C, Corado A L, Nascimento F. Silva G, Costa A. Duarte D, Pessoa K, Mejia M, et al. COVID-19 in Amazonas, Brazil, was driven by the persistence of endemic lineages and P.1 emergence. Nat Med. 2021; 27 (7): 1230-8.
12. Cherian S. Potdar V, Jadhav S, Yadav P. Gupta N, Das M, Rakshit P. Singh S, Abraham P. Panda S, et al. SARS-COV-2 Spike Mutations, L452R, T478K, E484Q and P681R, in the Second Wave of COVID-19 in Maharashtra, India. Microorganisms. 2021; 9 (7).
13. Garcia-Beltran W F. Lam E C, St Denis K, Nitido A D, Garcia Z H, Hauser B M, Feldman J. Pavlovic M N, Gregory D J, Poznansky M C, et al. Multiple SARS-COV-2 variants escape neutralization by vaccine-induced humoral immunity. Cell. 2021; 184 (9): 2523.
14. Zhou D, Dejnirattisai W, Supasa P. Liu C, Mentzer A J, Ginn H M, Zhao Y, Duyvesteyn H M E, Tuekprakhon A, Nutalai R, et al. Evidence of escape of SARS-COV-2 variant B.1.351 from natural and vaccine-induced sera. Cell. 2021; 184 (9): 2348-61 e6.
15. Hoffmann M, Arora P. Gross R. Seidel A, Hornich B F, Hahn A S, Kruger N, Graichen L, Hofmann-Winkler H, Kempf A, et al. SARS-COV-2 variants B.1.351 and P.1 escape from neutralizing antibodies. Cell. 2021; 184 (9): 2384-93 e12.
16. Planas D, Veyer D, Baidaliuk A, Staropoli I, Guivel-Benhassine F, Rajah M M, Planchais C, Porrot F. Robillard N, Puech J, et al. Reduced sensitivity of SARS-COV-2 variant Delta to antibody neutralization. Nature. 2021; 596 (7871): 276-80.
17. Collier D A, De Marco A, Ferreira I, Meng B, Datir R, Walls A C, Kemp S S, Bassi J, Pinto D, Fregni C S, et al. SARS-COV-2 B.1.1.7 sensitivity to mRNA vaccine-elicited, convalescent and monoclonal antibodies B, Bonsall D, Cicconi P, Charlton S, et al. Efficacy of ChAdOx1 nCOV-19 (AZD1222) vaccine against SARS-COV-2 variant of concern 202012/01 (B.1.1.7): an exploratory analysis of a randomised controlled trial. Lancet. 2021; 397 (10282): 1351-62.
19. Wibmer C K, Ayres F. Hermanus T, Madzivhandila M. Kgagudi P, Oosthuysen B, Lambson B E, de Oliveira T, Vermeulen M, van der Berg K, et al. SARS-COV-2 501Y.V2 escapes neutralization by South African COVID-19 donor plasma. Nat Med. 2021; 27 (4): 622-5.
20. Yu J. Tostanoski L H, Mercado N B, McMahan K, Liu J, Jacob-Dolan C. Chandrashekar A, Atyeo C, Martinez D R, Anioke T, et al. Protective efficacy of Ad26.COV2.S against SARS-COV-2 B.1.351 in macaques. Nature. 2021; 596 (7872): 423-7.
21. Corbett K S, Werner A P. S O C, Gagne M, Lai L, Moliva J I, Flynn B, Choi A, Koch M, Foulds K E, et al. Evaluation of mRNA-1273 against SARS-COV-2 B.1.351 Infection in Nonhuman Primates. bioRxiv. 2021.
22. Mlcochova P, Kemp S A, Dhar M S, Papa G, Meng B, Ferreira I, Datir R, Collier D A, Albecka A, Singh S, et al. SARS-COV-2 B.1.617.2 Delta variant replication and immune evasion. Nature. 2021; 599 (7883): 114-9.
23. Mizrahi B. Lotan R. Kalkstein N, Peretz A, Perez G, Ben-Tov A, Chodick G, Gazit S, and Patalon T. Correlation of SARS-COV-2-breakthrough infections to time-from-vaccine. Nat Commun. 2021; 12 (1): 6379.
24. Pegu A. O'Connell S E, Schmidt S D, O'Dell S, Talana C A, Lai L, Albert J, Anderson E. Bennett H, Corbett K S, et al. Durability of mRNA-1273 vaccine-induced antibodies against SARS-COV-2 variants. Science. 2021; 373 (6561): 1372-7.
25. Collier A Y, Yu J. McMahan K, Liu J, Chandrashekar A, Maron J S, Atyeo C. Martinez D R. Ansel J L, Aguayo R, et al. Differential Kinetics of Immune Responses Elicited by Covid-19 Vaccines. N Engl J Med. 2021.
26. Gagne M, Corbett K S, Flynn B J, Foulds K E, Wagner D A, Andrew S F, Todd J M, Honeycutt C C, McCormick L, Nurmukhambetova S T, et al. Protection from SARS-COV-2 Delta one year after mRNA-1273 vaccination in nonhuman primates is coincident with an anamnestic antibody response in the lower airway. bioRxiv. 2021.
27. Barouch D H, Stephenson K E, Sadoff J, Yu J, Chang A, Gebre M, McMahan K, Liu J, Chandrashekar A, Patel S, et al. Durable Humoral and Cellular Immune Responses 8 Months after Ad26.COV2.S Vaccination. N Engl J Med. 2021; 385 (10): 951-3.
28. Zabaleta N, Dai W. Bhatt U, Herate C. Maisonnasse P, Chichester J A, Sanmiguel J, Estelien R. Michalson K T, Diop C, et al. An AAV-based, room-temperature-stable, single-dose COVID-19 vaccine provides durable immunogenicity and protection in non-human primates. Cell Host Microbe. 2021; 29 (9): 1437-53 e8.
29. Lescure F X, Bouadma L, Nguyen D, Parisey M, Wicky P H, Behillil S, Gaymard A, Bouscambert-Duchamp M, Donati F, Le Hingrat Q, et al. Clinical and virological data of the first cases of COVID-19 in Europe: a case series. Lancet Infect Dis. 2020; 20 (6): 697-706.
30. Mori S. Wang L, Takeuchi T, and Kanda T. Two novel adeno-associated viruses from cynomolgus monkey: pseudotyping characterization of capsid protein. Virology. 2004; 330 (2): 375-83.
31. Mietzsch M, Jose A, Chipman P. Bhattacharya N, Daneshparvar N, McKenna R, and Agbandje-McKenna M. Completion of the AAV Structural Atlas: Serotype Capsid Structures Reveals Clade-Specific Features. Viruses. 2021; 13 (1).
32. Grieger J C, and Samulski R J. Packaging capacity of adeno-associated virus serotypes: impact of larger genomes on infectivity and postentry steps. J Virol. 2005; 79 (15): 9933-44.
33. Wu Z, Yang H, and Colosi P. Effect of genome size on AAV vector packaging. Mol Ther. 2010; 18 (1): 80-6.
34. Moyo-Gwete T, Madzivhandila M, Makhado Z, Ayres F. Mhlanga D, Oosthuysen B. Lambson B E, Kgagudi P. Tegally H, Iranzadeh A, et al. Cross-Reactive Neutralizing Antibody Responses Elicited by SARS-COV-2 501Y.V2 (B.1.351). N Engl J Med. 2021; 384 (22): 2161-3.
35. Liu C, Ginn H M, Dejnirattisai W, Supasa P, Wang B, Tuckprakhon A, Nutalai R, Zhou D. Mentzer A J, Zhao Y, et al. Reduced neutralization of SARS-COV-2 B.1.617 by vaccine and convalescent serum. Cell. 2021; 184 (16): 4220-36 e13.
36. Johnson M, Wagstaffe H R, Gilmour K C, Mai A L, Lewis J, Hunt A, Sirr J, Bengt C. Grandjean L, and Goldblatt D. Evaluation of a novel multiplexed assay for determining IgG levels and functional activity to SARS-COV-2. Journal of clinical virology: the official publication of the Pan American Society for Clinical Virology. 2020; 130 (104572.
37. Wang X. Yan Y, Gan T, Yang X, Li D, Zhou D, Sun Q. Huang Z, and Zhong J. A trivalent HCV vaccine elicits broad and synergistic polyclonal antibody response in mice and rhesus monkey. Gut. 2019; 68 (1).
38. Corman V M, Landt O, Kaiser M, Molenkamp R, Meijer A, Chu D K, Bleicker T, Brunink S. Schneider J, Schmidt M L, et al. Detection of 2019 novel coronavirus (2019-nCOV) by real-time RT-PCR. Euro surveillance: bulletin Europeen sur les maladies transmissibles=European communicable disease bulletin. 2020; 25 (3).
39. Wolfel R, Corman V M, Guggemos W. Seilmaier M, Zange S, Muller M A, Niemeyer D. Jones T C, Vollmar P. Rothe C, et al. Virological assessment of hospitalized patients with COVID-2019. Nature. 2020; 581 (7809): 465-9.
40. Zhang J, Binzel K, Miller M, Wright C, Siva A, Saif T, Knopp M, Maniawski P. Tung C-h, and Knopp M. Digital Photon Counting PET/CT: The Physics Powering Precision Nuclear Medicine. Journal of Nuclear Medicine. 2016; 57 (supplement 2): 1286-.
41. Shi H, Han X, Jiang N, Cao Y, Alwalid O, Gu J, Fan Y, and Zheng C. Radiological findings from 81 patients with COVID-19 pneumonia in Wuhan, China: a descriptive study. Lancet Infect Dis. 2020; 20 (4): 425-34.
42. Pan F. Ye T, Sun P, Gui S, Liang B, Li L, Zheng D, Wang J, Hesketh R L, Yang L, et al. Time Course of Lung Changes at Chest C T during Recovery from Coronavirus Disease 2019 (COVID-19). Radiology. 2020; 295 (3): 715-21.
43. Maisonnasse P, Guedj J, Contreras V. Bchillil S, Solas C, Marlin R, Naninck T. Pizzorno A, Lemaitre J, Goncalves A, et al. Hydroxychloroquine use against SARS-COV-2 infection in non-human primates. Nature. 2020; 585 (7826): 584-7.
44. Livak K, and Schmittgen T. Analysis of relative gene expression data using real-time quantitative PCR and the 2 (-Delta Delta C (T)) Method. Methods (San Diego, Calif). 2001; 25 (4).

### Example 2: Biodistribution, Cross-Neutralization and Immunogenicity

The following were performed as described in Materials and Methods unless otherwise noted:

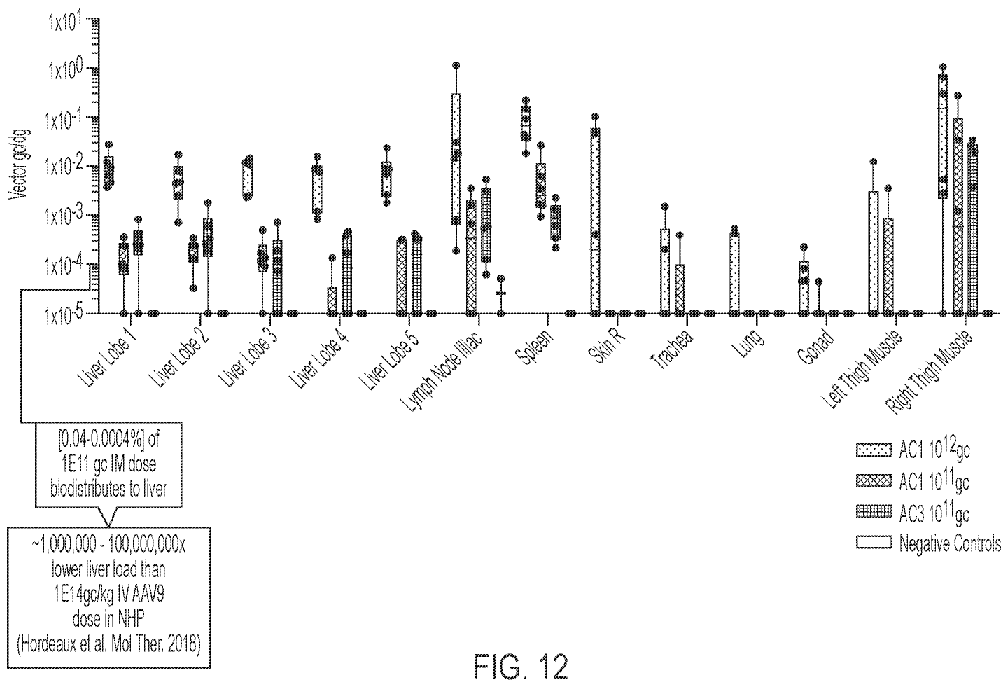

To evaluate the biodistribution of a low IM dose of CD platform, cynomolgus macaques (n=6) received single dose IM injection, at week 9 SARS-COV-2 1E5 PFU IN/IT, and necropsy at week 14 post immunization. As shown in FIG. 12, at proposed 1E11 gc dose, the primary distribution in NHP was focal, with biodistribution at the thigh muscle injection site and lymph node with limited distribution to liver.

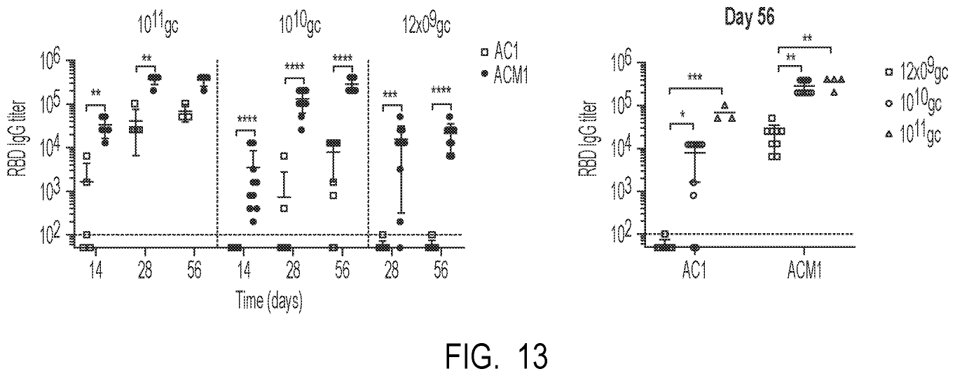

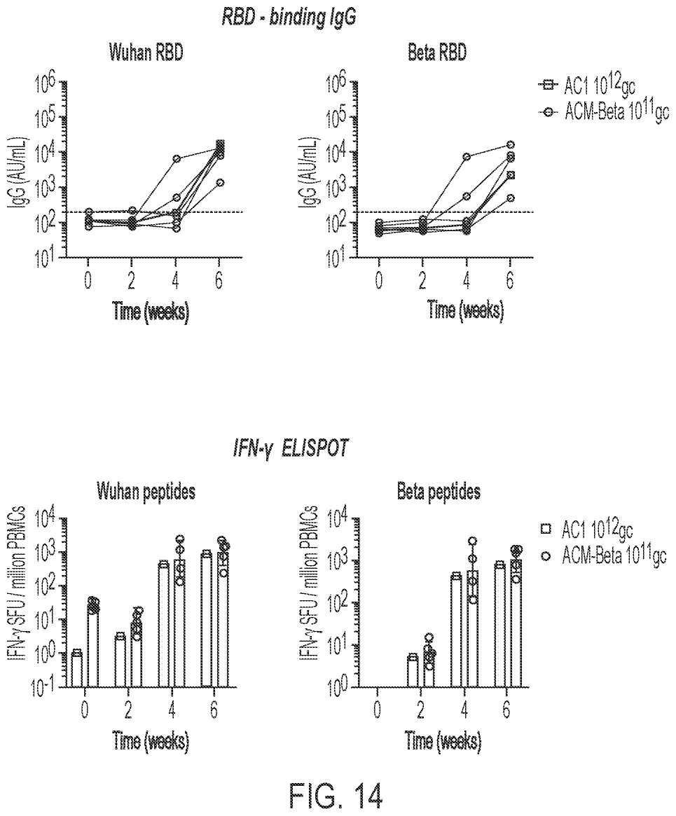

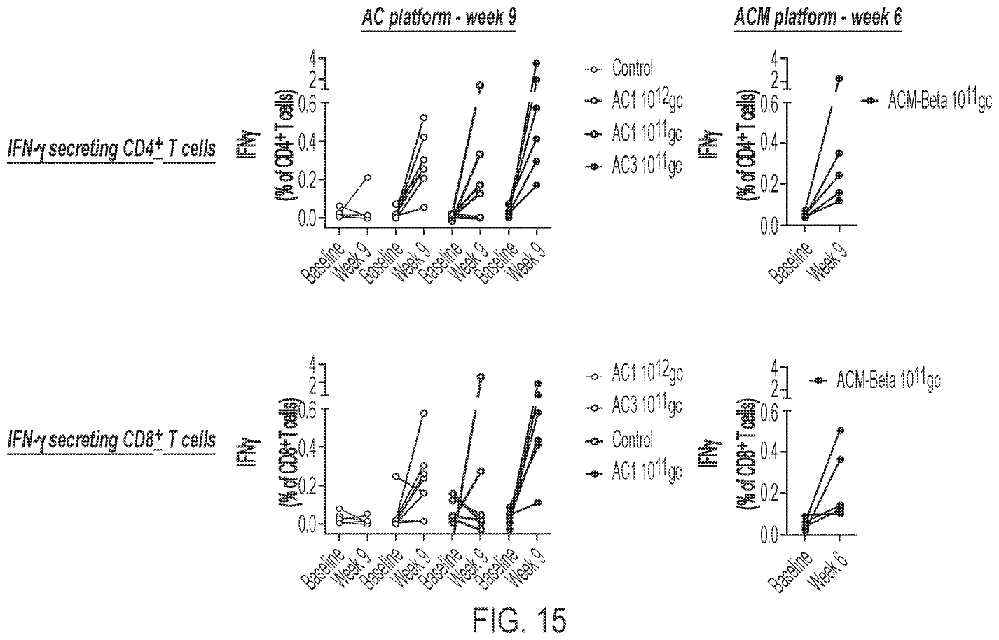

Cross-neutralization was measured by RBD-binding assay comparing AC1 and ACM1 over 100× range in C57BL/6 female mice. As shown in FIG. 13, ACM1 in C57BL/6 female mice elicited a faster and more potent response with a lower dose. VOC immunogenicity of ACM-Beta at 1011 gc was also tested. AC1 (with Spike Wuhan strain) was administered at 10× higher concentration than ACM-Beta, and results are shown in FIG. 14. Potent IFN-γ cellular responses were elicited in cynomolgus macaques using both the AC platform and ACM platform, as shown in FIG. 15.

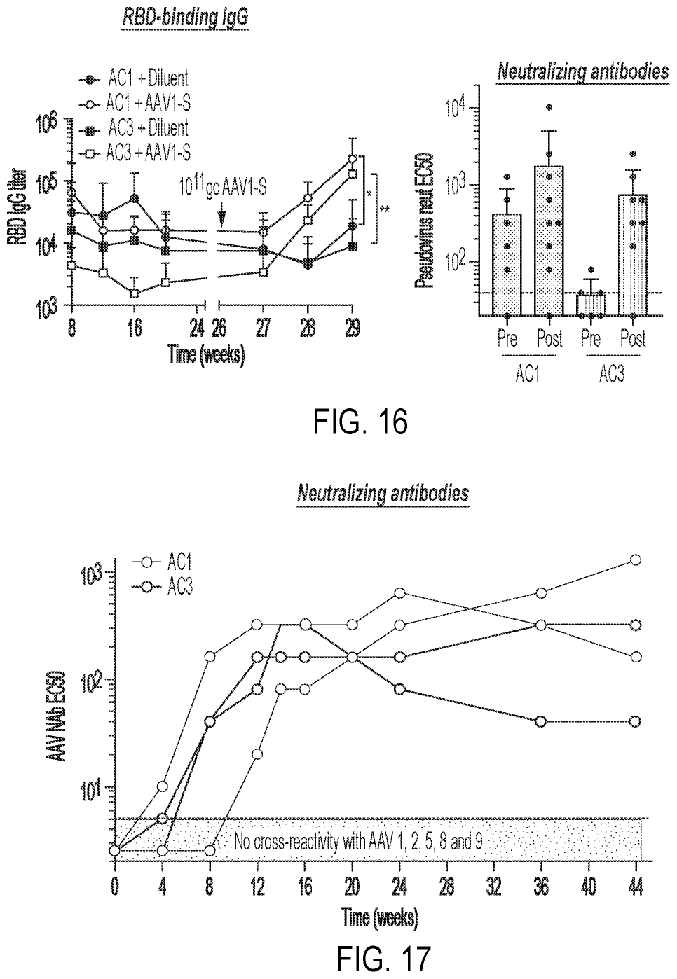

C57BL/6 female mice were also injected intramuscularly (IM) in the right gastrocnemius at week 0 with AC1 or AC3 1010 gc. Antibody responses were boosted at week 26 after vaccination with AAV1-SU.S. Plant Pat. No. 1011gc. Antibody responses increased after boosting with a different vector carrying the prefusion stabilized Spike. As shown in FIG. 16, desensitization against Spike antigen was observed.

As shown in FIG. 17, low anti-vector antibody responses were also obtained in rhesus. Slow kinetics of humoral responses against the vector were observed. Antibodies against AAVrh32.33 at 1E12 genome copies stabilized starting at week 12. No cross-reactivity was observed with serotypes 1, 2, 5, 8 and 9.

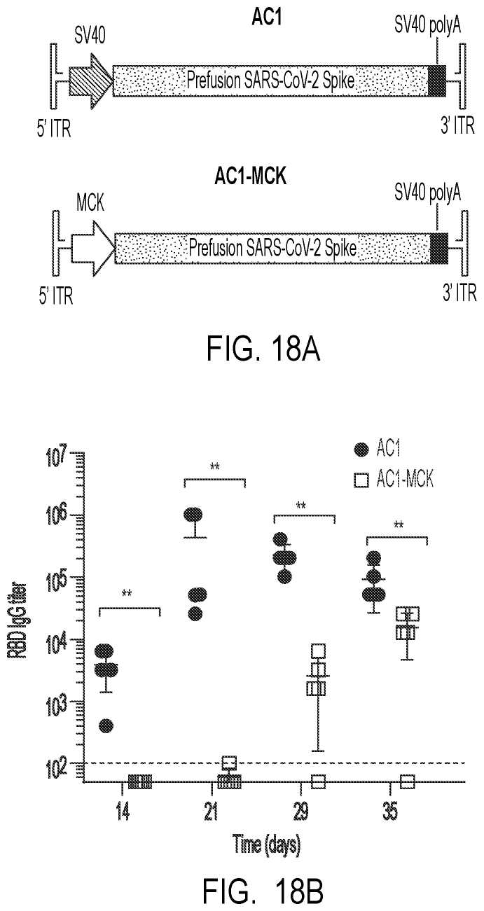

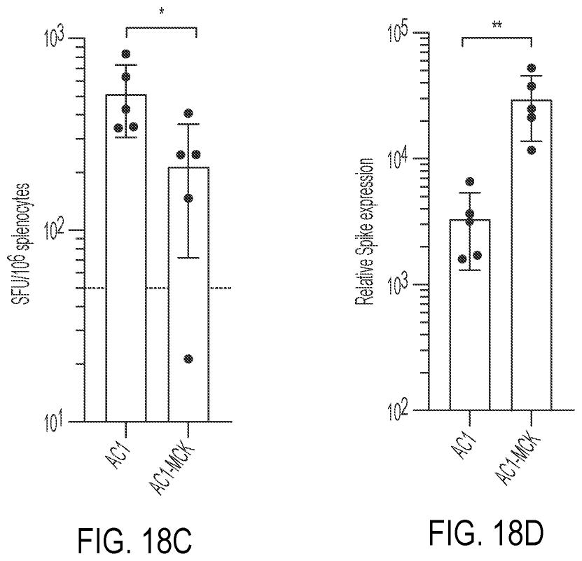

The immunogenicity of ubiquitous and muscle restricted AC1 vaccine was tested. The AC1 and AC1-MCK genomes are shown in FIG. 18A. Spike RBD binding IgG was tested in sera of C57BL/6 female mice vaccinated with 1011genome copiestotal dose of AC1 or AC1-MCK, as shown in FIG. 18B. IFN-γ secreting splenocytes were observed on day 35 (FIG. 18C). Transgene expression was observed in the injection site on day 7 after injection (FIG. 18D).

### Example 3: Viral Titration

AAV11 is produced in HEK293 cells via transient co-transfection of plasmids encoding all elements required for viral particle assembly. Briefly, HEK293 cells are grown to 90% confluency and transfected with (a) the viral genome plasmid encoding the luciferase transgene (expressed by the CMV promoter) flanked by AAV2 ITRs, (b) the AAV packaging plasmid encoding AAV2 rep and the synthesized capsid proteins disclosed herein, (c) AAV2-AAP expressing capsid, and (d) adenoviral helper genes needed for AAV packaging and assembly. Cells are incubated at 37° C. for 2 days, and cells and media are harvested and collected.

The cell-media suspension is lysed by three consecutive freeze-thaw cycles. Next, the lysate is cleared by centrifugation and treated with an enzyme under conditions to perform exhaustive DNA digestion (e.g., with Benzonase™) to digest any DNA present outside of the virus particle. The AAV preparation is diluted to fall within the linear measurement range of a control DNA template, in this case linearized plasmid with identical TaqMan™ primer and probe binding sequence as compared to the vector genome. TaqMan™ PCR is performed with primers and probe annealing to the viral vector genome of choice. Titer is calculated based on the TaqMan™ measurement in genome copies (GC) per milliliter (ml).

### Example 4: Neutralizing Antibody Assay

Neutralizing antibody assays are performed to evaluate how resistant AAV11 is to antibody-neutralization. Neutralizing antibody assays measure the antibody concentration (or the titer at which an experimental sample contains an antibody concentration) that neutralizes an infection by 50% or more as compared to a control in the absence of the antibody.

Serum samples or IVIG stock solution (200 mg/ml) are serially diluted by 2-fold, and undiluted and diluted samples are co-incubated with AAV11 at a MOI of 10c4 for about 30 minutes at 37° C. The AAV11 included a luciferase transgene. The admixed vector and an antibody sample then are transduced into HEK293 cells. For these experiments, the antibody sample used is intravenous immunoglobulin (IVIG), pooled IgGs extracted from the plasma of over one thousand blood donors (sold commercially, for example, as Gammagard™ (Baxter Healthcare; Deerfield, IL) or Gamunex™ (Grifols; Los Angeles, CA)). 48 hours following initiation of transduction, cells are assayed by bioluminescence to detect luciferase. Neutralizing antibody titer is determined by identifying the dilution of sample for which 50% or more neutralization (transduction of sample/transduction of control virus in absence of sample) is reached.

High concentrations of IVIG are required to reduce the transduction efficiency of AAV11 to below 50% of a no-IVIG control compared to, for example, AAV2. These results demonstrate higher resistance of AAV11 to neutralization by IVIG as compared to, for example, AAV2.

### Example 5: In Vivo Vaccinations

Vaccinations are performed to determine whether or not AAV11 vectors are able to deliver an immunogen to cells in vivo. 2×10e8 genome copies (GC) of AAV11, which includes an eGFP-encoding transgene, are intravenously administered to mice. GFP expression is monitored non-invasively by fundus photography of the animal. All of the animals demonstrate varying degrees of successful targeting of AAV11 to cells.

### Example 6: Measuring Immunogenicity Against SARS-COV-2 Full Length Stabilized Spike in Mice Vaccinated with AAV-COVID AAV11

Female BALB/c mice (N=5) were injected intramuscularly (IM) with 1×10e10 or 1×10e11 gc/vector of AAV-COVID AAV11 or Rh32.33 (B857X). Sera was collected just before injection (baseline), and at day 14 (D14), day 21 (D21) and day 28 (D28) to evaluate the humoral response. ELIspot biodistribution data for the right gastroc, liver and spleen were obtained for both doses.

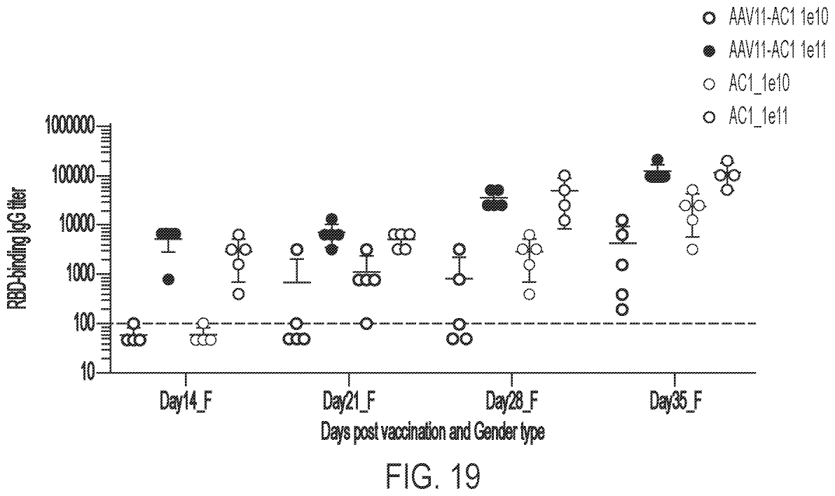

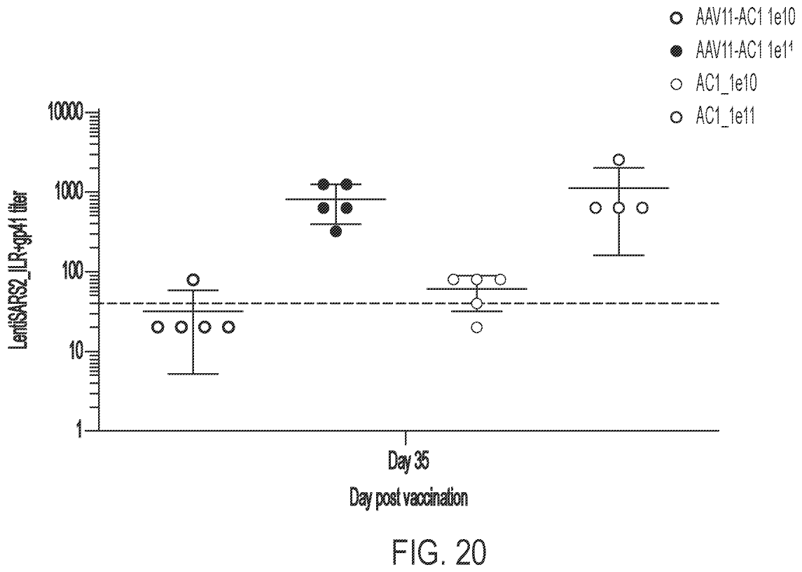

AAV11-AC1 elicited similar levels of dose and time-dependent RBD-specific IgG antibodies (FIG. 19) and spike neutralizing antibodies (FIG. 20) as compared to Rh32.33-AC1 in female Balb/c mice.

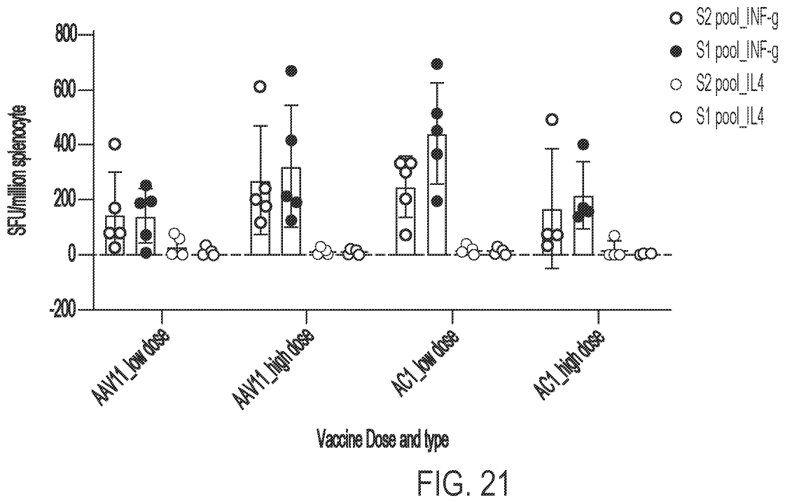

AAV11-AC1 elicited similar levels of INF-gamma and IL-4-secreting T cells as compared to Rh32.33-AC1 at both doses in female Balb/c mice strain when stimulated with SARS-COV-2 S1 and S2 peptide pools. FIG. 21.

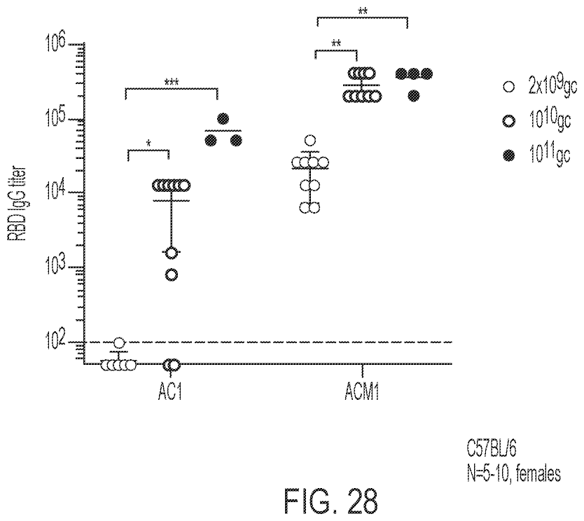

Female C57BL/6 mice (N=5-10) were injected intramuscularly (IM) with 2×10e9, 1×10e10 or 1×10e11 gc/vector of AAV-COVID AAV11 under direction of the mini CMV promoter (ACM) or Rh32.33 with the SV40 promoter (AC1). Sera was collected at day 56 to evaluate the humoral response. Significantly, ACM elicited at least 10-times higher levels of RBD-specific IgG antibodies did AC1 in all dose to dose comparisons or, alternatively, the same level of RBD-specific IgG antibodies was produced at a 50-times lower dose than AC1 (FIG. 28) in female mice.

### Example 7: Measuring Immunogenicity Against SARS-COV-2 Full Length Stabilized Spike in C57BL/6 Mice Vaccinated with AAV-COVID AAV11

Male (N=5) and female (N=5) C57BL/6 mice were injected intramuscularly (IM) with 1×10e10 or 1×10e11 gc/vector of AAV-COVID AAV11 or Rh32.33 at day 0. Sera was collected just before injection (baseline), and at day 14 (D14), day 28 (D28), day 42 (D42), day 56 (D56) and day 71 (D71), when they were sacrificed, to evaluate the humoral response. ELIspot data from T cells as well as for the right gastroc, liver and spleen were obtained for the low dose experiments, while full biodistribution ELIspot data were obtained for the high dose experiments.

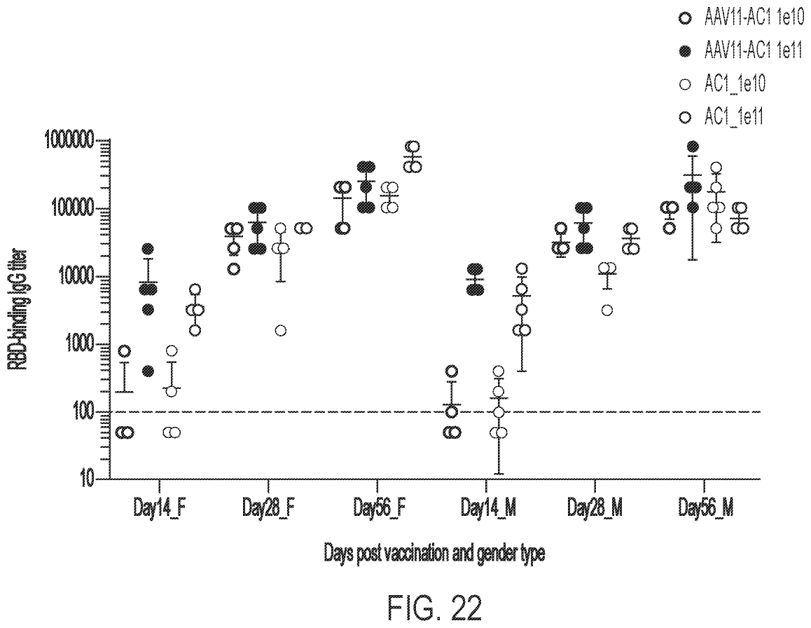

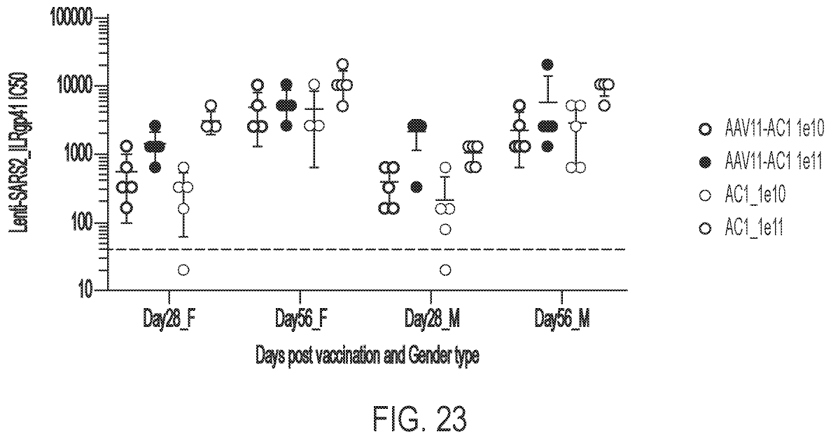

AAV11-AC1 elicited similar levels of dose and time-dependent RBD-specific IgG antibodies (FIG. 22) and spike neutralizing antibodies (FIG. 23) at both doses in both genders of C57BL/6 mice relative to Rh32.33-AC1.

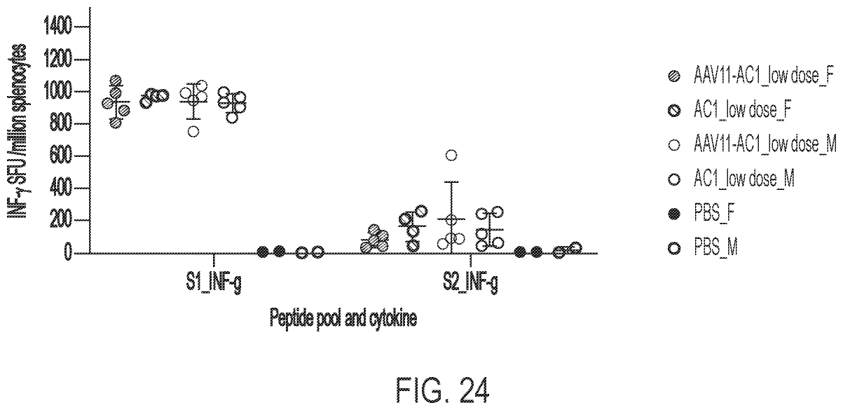

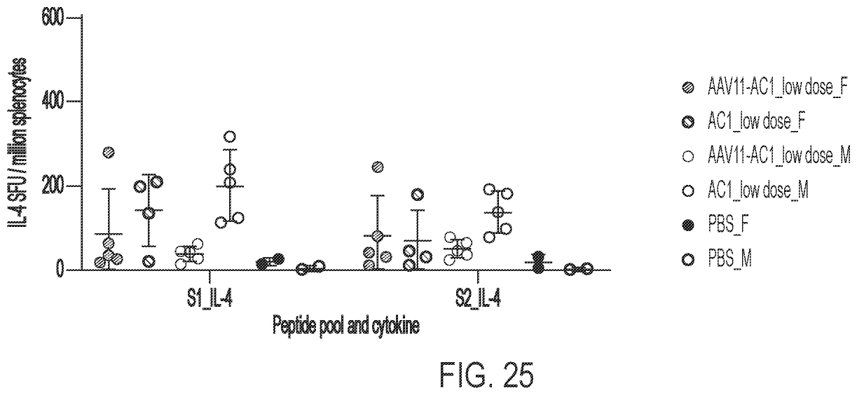

At the low dose (i.e., 1×10e10 gc/vector), AAV11-AC1 elicited similar levels of INF-gamma secreting-T-cells (FIG. 24) but lower levels of IL-4 secreting T-cells (FIG. 25) relative to Rh32.33-AC1 in both genders of C57BL/6 mice when stimulated with SARS-COV-2 S1 and S2 peptide pools.

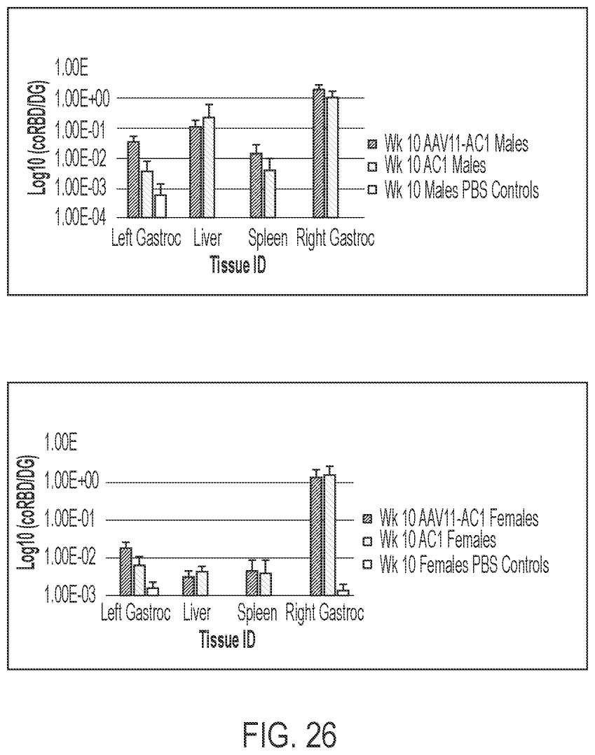

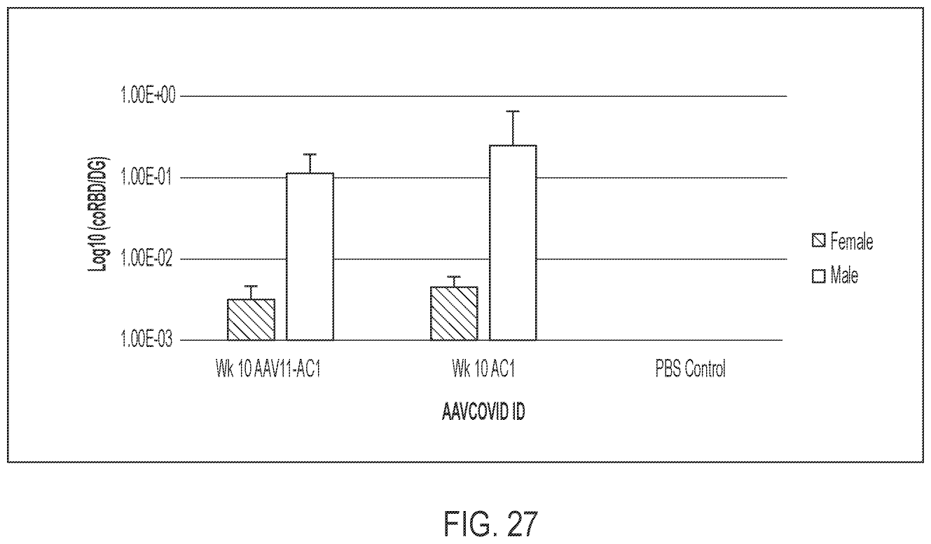

At the high dose (i.e., 1×10e11 gc/vector), similar patterns of bio-distribution were observed for AAV11-AC1 and Rh32.33-AC1 in both genders of C57BL/6 mice (FIG. 26), however, gender disparity in liver biodistribution was observed for both AAV11-AC1 and Rh32.3-AC1, with females having a lower number of genomes than males (FIG. 27).

### Example 8: Upper Respiratory Tract Protection in Non-Human Primates

Cynomolgus macaques (Macaca fascicularis) (N=6) were injected intramuscularly (IM) with 1×10e12 gc/vector of AC1 or 1×10e11 gc/vector of ACM-beta at day 0.

Immunogenicity readouts, including RBD and Spike binding IgGs, RBD and Spike ACE2 binding inhibition, pseudovirus neutralization and IFN-Î3 ELISpot, were obtained at approximately day 26.

At approximately day 54, each macaques was challenged with 105 pfu SARS-COV-2 Beta VOC intranasally or intratracheally. Protection was evaluated based on the amount of viral genomic and subgenomic RNA present, CT and histology.

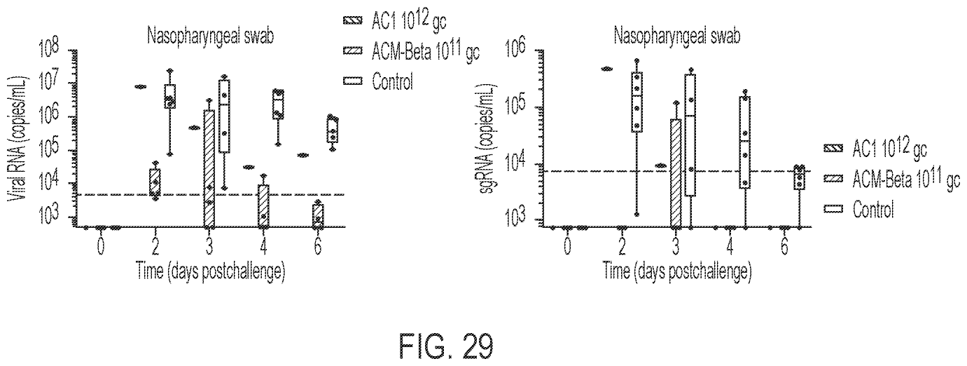

These experiments demonstrated protection by ACM at a dose of 1×10e11, whereas the AC1 high dose (N=1) did not provide protection (FIG. 29). In addition, viral genomes could be detected in 4 out of 5 animals on day 2, which is very close to the LOQ, and one breakthrough was detected based on the presence of subgenomic DNA.

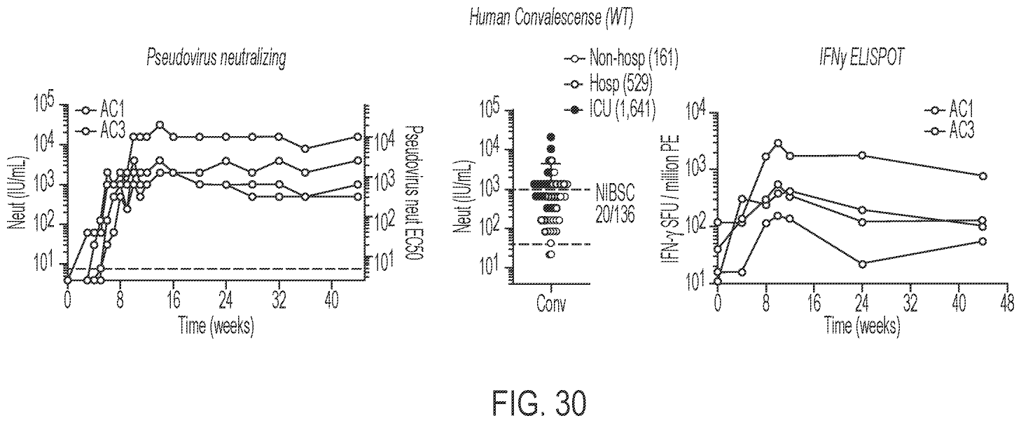

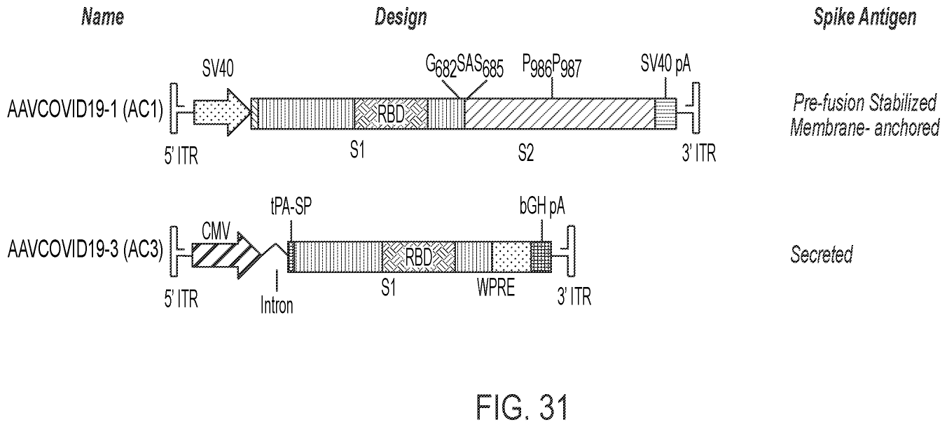

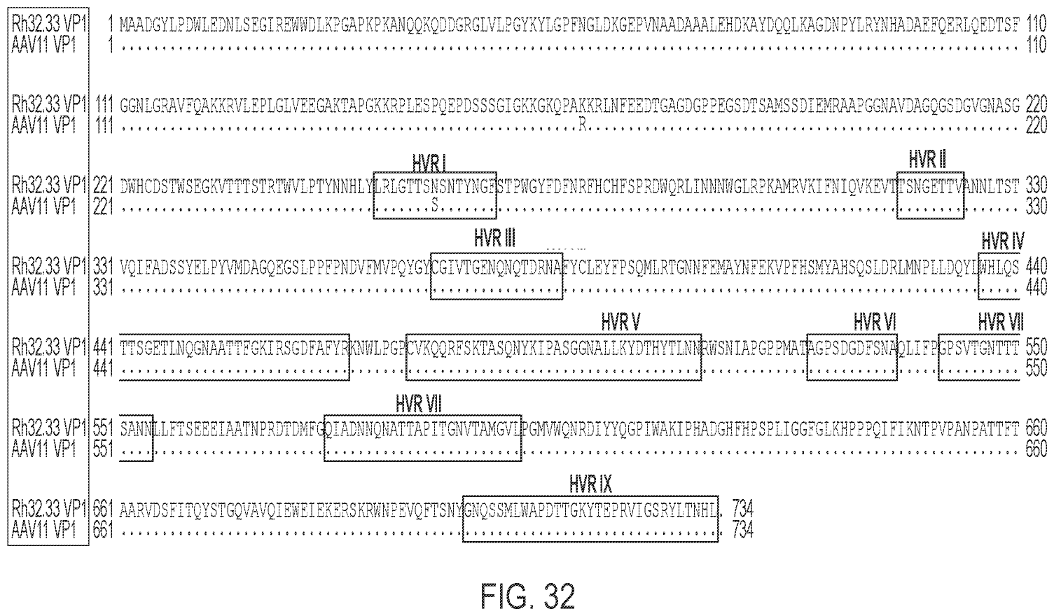

Rhesus macaques (N=2; 1 male, 1 female) were vaccinated intramuscularly (IM) with 1×10e12 gc/vector of AC1 and AC3. The durability of immunogenicity was evaluated after 11 months, including extent of neutralization (left) compared with human convalescence (WT) data (center) and IFN-gamma EliSPOT (right) (FIGS. 30-32).

### Example 9: The Sequence of the AAV11 Capsid Protein

(SEQ ID NO: 1)
MAADGYLPDW LEDNLSEGIR EWWDLKPGAP KPKANQQKOD DGRGLVLPGY KYLGPFNGLD
KGEPVNAADA AALEHDKAYD QQLKAGDNPY LRYNHADAEF QERLQEDTSF GGNLGRAVFQ
AKKRVLEPLG LVEEGAKTAP GKKRPLESPQ EPDSSSGIGK KGKQPARKRL NFEEDTGAGD
GPPEGSDISA MSSDIEMRAA PGGNAVDAGQ GSDGVGNASG DWHCDSTWSE GKVTTTSTRT
WVLPTYNNHL YLRLGTTSSS NTYNGFSTPW GYFDFNRFHC HFSPRDWQRL INNNWGLRPK
AMRVKIFNIQ VKEVTTSNGE TTVANNLTST VQIFADSSYE LPYVMDAGQE GSLPPFPNDV
FMVPQYGYCG IVTGENQNQT DRNAFYCLEY FPSQMLRTGN NFEMAYNFEK VPFHSMYAHS
QSLDRLMNPL LDQYLWHLQS TTSGETLNQG NAATTFGKIR SGDFAFYRKN WLPGPCVKQQ
RFSKTASQNY KIPASGGNAL LKYDTHYTLN NRWSNIAPGP PMATAGPSDG DFSNAQLIFP
GPSVTGNTTT SANNLLFTSE EEIAATNPRD TDMFGQIADN NQNATTAPIT GNVTAMGVLP
GMVWQNRDIY YQGPIWAKIP HADGHFHPSP LIGGFGLKHP PPQIFIKNTP VPANPATIFT
AARVDSFITQ YSTGQVAVQI EWEIEKERSK RWNPEVQFTS NYGNQSSMLW APDTTGKYTE
PRVIGSRYLI NHL
(SEQ ID NO: 2)
ATGGCTGCTG ACGGTTATCT TCCAGATTGG CTCGAGGACA ACCTCTCTGA GGGCATTCGC
GAGTGGTGGG ACCTGAAACC TGGAGCCCCG AAGCCCAAGG CCAACCAGCA GAAGCAGGAC
GACGGCCGGG GTCTGGTGCT TCCTGGCTAC AAGTACCTCG GACCCTTCAA CGGACTCGAC
AAGGGGGAGC CCGTCAACGC GGCGGACGCA GCGGCCCTCG AGCACGACAA GGCCTACGAC
CAGCAGCTCA AAGCGGGTGA CAATCCGTAC CTGCGGTATA ACCACGCCGA CGCCGAGTTT
CAGGAGCGTC TGCAAGAAGA TACGTCTTTT GGGGGCAACC TCGGGCGAGC AGTCTTCCAG
GCCAAGAAGA GGGTACTCGA ACCTCTGGGC CTGGTTGAAG AAGGTGCTAA AACGGCTCCT
GGAAAGAAGA GACCGTTAGA GTCACCACAA GAGCCCGACT CCTCCTCGGG CATCGGCAAA
AAAGGCAAAC AACCAGCCAG AAAGAGGCTC AACTTTGAAG AGGACACTGG AGCCGGAGAC
GGACCCCCTG AAGGATCAGA TACCAGCGCC ATGTCTTCAG ACATTGAAAT GCGTGCAGCA
CCGGGCGGAA ATGCTGTCGA TGCGGGACAA GGTTCCGATG GAGTGGGTAA TGCCTCGGGT
GATTGGCATT GCGATTCCAC CTGGTCTGAG GGCAAGGTCA CAACAACCTC GACCAGAACC
TGGGTCTTGC CCACCTACAA CAACCACTTG TACCTGCGTC TCGGAACAAC ATCAAGCAGC
AACACCTACA ACGGATTCTC CACCCCCTGG GGATATTTTG ACTTCAACAG ATTCCACTGT
CACTTCTCAC CACGTGACTG GCAAAGACTC ATCAACAACA ACTGGGGACT ACGACCAAAA
GCCATGCGCG TTAAAATCTT CAATATCCAA GTTAAGGAGG TCACAACGTC GAACGGCGAG
ACTACGGTCG CTAATAACCT TACCAGCACG GTTCAGATAT TTGCGGACTC GTCGTATGAG
CTCCCGTACG TGATGGACGC TGGACAAGAG GGGAGCCTGC CTCCTTTCCC CAATGACGTG
TTCATGGTGC CTCAATATGG CTACTGTGGC ATCGTGACTG GCGAGAATCA GAACCAAACG
GACAGAAACG CTTTCTACTG CCTGGAGTAT TTTCCTTCGC AAATGTTGAG AACTGGCAAC
AACTTTGAAA TGGCTTACAA CTTTGAGAAG GTGCCGTTCC ACTCAATGTA TGCTCACAGC
CAGAGCCTGG ACAGACTGAT GAATCCCCTC CTGGACCAGT ACCTGTGGCA CTTACAGTCG
ACTACCTCTG GAGAGACTCT GAATCAAGGC AATGCAGCAA CCACATTTGG AAAAATCAGG
AGTGGAGACT TTGCCTTTTA CAGAAAGAAC TGGCTGCCTG GGCCTTGTGT TAAACAGCAG
AGATTCTCAA AAACTGCCAG TCAAAATTAC AAGATTCCTG CCAGCGGGGG CAACGCTCTG
TTAAAGTATG ACACCCACTA TACCTTAAAC AACCGCTGGA GCAACATCGC GCCCGGACCT
CCAATGGCCA CAGCCGGACC TTCGGATGGG GACTTCAGTA ACGCCCAGCT TATATTCCCT
GGACCATCTG TTACCGGAAA TACAACAACT TCAGCCAACA ATCTGTTGTT TACATCAGAA
GAAGAAATTG CTGCCACCAA CCCAAGAGAC ACGGACATGT TTGGCCAGAT TGCTGACAAT
AATCAGAATG CTACAACTGC TCCCATAACC GGCAACGTGA CTGCTATGGG AGTGCTGCCT
GGCATGGTGT GGCAAAACAG AGACATTTAC TACCAAGGGC CAATTTGGGC CAAGATCCCA
CACGCGGACG GACATTTTCA TCCTTCACCG CTGATTGGTG GGTTTGGACT GAAACACCCG
CCTCCCCAGA TATTCATCAA GAACACTCCC GTACCTGCCA ATCCTGCGAC AACCTTCACT
GCAGCCAGAG TGGACTCTTT CATCACACAA TACAGCACCG GCCAGGTCGC TGTTCAGATT
GAATGGGAAA TTGAAAAGGA ACGCTCCAAA CGCTGGAATC CTGAAGTGCA GTTTACTTCA
AACTATGGGA ACCAGTCTTC TATGTTGTGG GCTCCTGATA CAACTGGGAA GTATACAGAG
CCGCGGGTTA TTGGCTCTCG TTATTTGACT AATCATTTGT AA

### EQUIVALENTS AND SCOPE

Those skilled in the art will recognize or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An AAV11 vector comprising at least one AAV11 capsid protein and a nucleic acid encoding a transgene operably linked to a promoter, wherein the transgene encodes an immunogenic polypeptide.

2. The AAV11 vector of claim 1, wherein the nucleic acid further comprises other regulatory elements.

3. The AAV11 vector of claim 2, wherein the other regulatory elements comprise a Woodchuck Hepatitis Virus (WHP) Posttranscriptional Regulatory Element (WPRE).

4. The AAV11 vector of any of the preceding claims, wherein the nucleic acid further comprises a polyadenylation (polyA) sequence.

5. The AAV11 vector of claim 4, wherein the polyA sequence is a short synthetic polyA (SPA) sequence.

6. The AAV11 vector of any one of the preceding claims, wherein the immunogenic polypeptide is from a virus selected from the group consisting of Adenoviruses, Arenaviruses, Arteriviruses, Birnaviruses, Bunyaviruses, Caliciviruses, Coronaviruses, Filoviruses, Flaviviruses, Hepadnaviruses, Herpesviruses, Human immunodeficiency virus (HIV), Human papilloma virus (HPV), Orthomyxoviruses, Paramyxoviruses, Picornaviruses, Parvoviruses, Papovaviruses, Poxviruses, Rhabdoviruses, Reoviruses, Retroviruses, and Togaviruses.

7. The AAV11 vector of any of claims 1-5, wherein the immunogenic polypeptide is from a microorganism selected from a species selected from Acinetobacter spp., Bacillus spp., Bartonella spp., Bordetella spp., Borelia spp., Brucella spp., Camplybacter spp., Chlamydia spp., Clostridium spp., Corynebacterium spp., Escherichia spp., Ehrlichia spp., Enterococcus spp., Enterococcus spp., Francisella spp., Haemophilus spp., Helicobacter spp., Klebsiella spp., Legionella spp., Leptospira spp., Listeria spp., Mycobacterium spp., Mycoplasma spp., Neisseria spp., Parachlamydia spp., Salmonella spp., Shigella spp., Staphylococcus spp., Streptococcus spp., Vibrio spp., and Yersinia spp.

8. The AAV11 vector of any of claims 1-6, wherein the immunogenic polypeptide comprises a viral antigen, a bacterial antigen, a parasitic antigen, or a fungal antigen.

9. The AAV11 vector of claim 8, wherein the viral antigen comprises a coronavirus spike protein or a fragment of a coronavirus spike protein.

10. The AAV11 vector of claim 9, wherein the viral antigen comprises a coronavirus receptor-binding domain (RBD) or a fragment of a coronavirus RBD.

11. The AAV11 vector of either claim 9 or 10, wherein the coronavirus is a SARS-COV-2 virus.

12. The AAV11 vector of any of claims 1-5, wherein the immunogenic polypeptide comprises a cancer antigen.

13. The AAV11 vector of claim 12, wherein the cancer antigen is NY-ESO-1, 10 HER2, HPV16 E7, CEA, WT1, MART-1, gp100, tyrosinase, URLC10, VEGFR1, VEGFR2, survivin, MUC1, or MUC2.

14. The AAV11 vector of any one of the preceding claims, wherein the promoter is selected from the group consisting of a CAG promoter, an EF1 alpha promoter, a p5 promoter, a p19 promoter, a p40 promoter, a SV40 promoter, an elongation factor short (EFS) promoter, a muscle creatine kinase (MCK) promoter, a cytomegalovirus (CMV) promoter, or a minimal CMV (mini-CMV) promoter.

15. The AAV11 vector of any one of the preceding claims, wherein the nucleic acid further comprises an intron.

16. The AAV11 vector of claim 15 wherein the intron is located downstream of the promoter.

17. The AAV11 vector of claim 14, wherein the promoter is a CMV promoter and the nucleic acid further comprises an intron located downstream of the promoter.

18. The AAV11 vector of either claim 14 or 17 wherein the promoter is a mini-CMV promoter.

19. The AAV11 vector of claim 18, wherein the mini-CMV promoter has at least 90% identity to SEQ ID NO: 12.

20. The AAV11 vector of claim 18, wherein the mini-CMV promoter is SEQ ID NO: 12.

21. The AAV11 vector of any one of the preceding claims, wherein the AAV11 viral capsid protein has an amino acid sequence at least 95% identical to SEQ ID NO: 1.

22. The AAV11 vector of claim 21, wherein the AAV11 viral capsid protein has an amino acid sequence that is SEQ ID NO: 1.

23. The AAV11 vector of claim 21, wherein the AAV11 viral capsid protein has an amino acid sequence comprising at least 2 amino acid substitutions compared to SEQ ID NO: 1.

24. The AAV11 vector of claim 21, wherein the AAV11 viral capsid protein has an amino acid sequence comprising at least 2 amino acid deletions compared to SEQ ID NO: 1.

25. The AAV11 vector of claim 21, wherein the AAV11 viral capsid protein has an amino acid sequence comprising 7 amino acid insertions compared to SEQ ID NO: 1.

26. A composition comprising the AAV11 vector of any one of the preceding claims, and a pharmaceutically acceptable carrier.

27. A method of eliciting an immune response in a subject comprising administering to the subject the composition of claim 26.

28. A method of treating or preventing a disease in a subject comprising administering to the subject the composition of claim 26.

29. The method of claim 27 or 28, wherein the composition is administered to the subject only once.

30. The method of claim 27 or 28, wherein the composition is administered to the subject more than once.

31. The method of any one of claims 27-30, wherein the composition administered to the subject comprises a viral dosage of 10⁸ to 10¹³ genome copies.

32. The method of any one of claims 27-31, wherein the composition is administered to the subject via a route of administration selected from the group consisting of intramuscular, intravenous, subcutaneous, rectal, intravaginal, parenteral, oral, sublingual, intratracheal, and intranasal.

33. The method of any one of claims 27-32, wherein the subject is a mammal.

34. The method of any one of claims 27-33, wherein the subject is selected from the group consisting of a human, a non-human primate, a rodent, an exotic animal, a companion animal, and livestock.

35. The method of any one of claims HEK293 cells27-34, wherein the subject is at risk of developing an infection or cancer.

36. The method of claim 35, wherein the subject is at risk of developing a disease selected from the group consisting of SARS-COV-1 and SARS-COV-2 (COVID-19).

37. A method of manufacturing an AAV11 vector, comprising
(i) transfecting a producer cell with a nucleic encoding an AAV11 capsid protein and another nucleic acid encoding an immunogenic polypeptide operably linked to a promoter, and
(ii) culturing the producer cell under conditions in which AAV11 vectors comprising at least one AAV11 viral capsid protein carrying the nucleic acid are produced.

38. The method of claim 37, wherein the immunogenic polypeptide is from a virus selected from the group consisting of Adenoviruses, Arenaviruses, Arteriviruses, Birnaviruses, Bunyaviruses, Caliciviruses, Coronaviruses, Filoviruses, Flaviviruses, Hepadnaviruses, Herpesviruses, Human immunodeficiency virus (HIV), Human papilloma virus (HPV), Orthomyxoviruses, Paramyxoviruses, Picornaviruses, Parvoviruses, Papovaviruses, Poxviruses, Rhabdoviruses, Reoviruses, Retroviruses, and Togaviruses.

39. The method of claim 37, wherein the immunogenic polypeptide is from a microorganism selected from a species selected from Acinetobacter spp., Bacillus spp., Bartonella spp., Bordetella spp., Borelia spp., Brucella spp., Camplybacter spp., Chlamydia spp., Clostridium spp., Corynebacterium spp., Escherichia spp., Ehrlichia spp., Enterococcus spp., Enterococcus spp., Francisella spp., Haemophilus spp., Helicobacter spp., Klebsiella spp., Legionella spp., Leptospira spp., Listeria spp., Mycobacterium spp., Mycoplasma spp., Neisseria spp., Parachlamydia spp., Salmonella spp., Shigella spp., Staphylococcus spp., Streptococcus spp., Vibrio spp., and Yersinia spp.

40. The method of claim 37, wherein the immunogenic polypeptide comprises a viral antigen, a bacterial antigen, a parasitic antigen, or a fungal antigen.

41. The method of claim 37, wherein the immunogenic polypeptide comprises a cancer antigen.

42. The method of claim 41, wherein the polypeptide is selected from the group consisting of NY-ESO-1, 10 HER2, HPV16 E7, CEA, WT1, MART-1, gp100, tyrosinase, URLC10, VEGFR1, VEGFR2, survivin, MUC1, and MUC2.

43. The method of any one of claims 37-42, wherein the AAV11 viral capsid protein has an amino acid sequence that is at least 95% identical to SEQ ID NO: 1.

44. The method of any one of claims 37-42, wherein the AAV11 viral capsid protein has an amino acid sequence that is SEQ ID NO: 1.

45. The method of any one of claims 37-42, wherein the AAV11 viral capsid protein has an amino acid sequence comprising at least 2 amino acid substitutions compared to SEQ ID NO: 1.

46. The method of any one of claims 37-42, wherein the AAV11 viral capsid protein has an amino acid sequence comprising at least 2 amino acid deletions compared to SEQ ID NO: 1.

47. The AAV11 vector of claim 37-42, wherein the AAV11 viral capsid protein has an amino acid sequence comprising 7 amino acid insertions compared to SEQ ID NO: 1.

48. The method of any one of claims 37-47, wherein the antigen plasmid further comprises a Woodchuck Hepatitis Virus (WHP) Posttranscriptional Regulatory Element (WPRE).

49. The method of any one of claims 37-48, wherein the viral antigen comprises a coronavirus spike protein, or a fragment of a coronavirus spike protein.

50. The method of claim 49, wherein the viral antigen comprises a coronavirus receptor-binding domain (RBD), or a fragment of a coronavirus RBD.

51. The method of any one of claims 37-50, wherein the coronavirus is a SARS-COV-2 virus.

52. The method of any one of claims 37-51, wherein the promoter is selected from the group consisting of a CAG promoter, an EF1 alpha promoter, a p5 promoter, a p19 promoter, a p40 promoter, a SV40 promoter, an elongation factor short (EFS) promoter, a muscle creatine kinase (MCK) promoter, a cytomegalovirus (CMV) promoter, and a minimal CMV (mini-CMV) promoter.

53. The method of claim 37, wherein the antigen plasmid comprises a nucleic acid sequence at least 90% identical to any one of SEQ ID NOs: 3-6.

54. The method of claim 37, wherein the antigen plasmid comprises a nucleic acid sequence that is any one of SEQ ID NOs: 3-6.

55. The method of any one of claims 37-54, wherein the cell is an insect cell.

56. The method of claim 55, wherein the insect cell is a baculovirus cell.

57. The method of any one of claims 37-54, wherein the cell is a mammalian cell.

58. The method of any one of claims 37-57, wherein the nucleic acid encoding an immunogenic polypeptide is expressed from a recombinant AAV genome.

59. A vaccine comprising the AAV11 vector of any one of claims 1-25.
